(19) United States
(12) Patent Application Publication
(10) Pub. No.: US 2026/0232691 A1
(43) Pub. Date: Aug. 13, 2026

(54) NBD1 MODULATORS AND METHODS OF USING THE SAME

(71) Applicants: Sionna Therapeutics Inc., Waltham, MA (US); Genzyme Corporation, Cambridge, MA (US)

(72) Inventors: Junkai Liao, Andover, MA (US); Mark Munson, Acton, MA (US); Sukanthini Thurairatnam, Dover, MA (US); Bradford Hirth, Littleton, MA (US); Zhongli Gao, Waltham, MA (US); Gregory Donald Hurlbut, Boxborough, MA (US); Jinyu Liu, Union City, CA (US); Michael Kothe, Medway, MA (US); George Topalov, Pittsburgh, PA (US); John E. Macor, Washington Crossing, PA (US); Yi Li, Weston, MA (US); Andrew Good, Bideford (GB)

(21) Appl. No.: 19/160,775

(22) PCT Filed: Mar. 1, 2024
(86) PCT No.: PCT/US2024/018148

§ 371 Date: Aug. 28, 2025

Related U.S. Application Data
(60) Provisional application No. 63/449,472, filed on Mar. 2, 2023.

(51) Int. Cl. A61K 31/553; A61K 31/4196; A61K 31/4745; C07D 471/04; C07D 487/04; C07D 498/04

(52) U.S. Cl. CPC A61K 31/553; A61K 31/4196; A61K 31/4745; C07D 471/04; C07D 487/04; C07D 498/04

(57) ABSTRACT

The present disclosure includes, among other things, CFTR modulators, pharmaceutical compositions, and methods of making and using the same.

This application claims the benefit of and priority to U.S. Provisional Patent Application No. 63/449,472, filed on Mar. 2, 2023, the disclosure of each of which is hereby incorporated by reference in its entirety for all purposes.

### BACKGROUND

Cystic fibrosis (CF), an autosomal recessive disorder, is caused by functional deficiency of the cAMP-activated plasma membrane chloride channel, cystic fibrosis transmembrane conductance regulator (CFTR), which results in pulmonary and other complications. The gene encoding CFTR has been identified and sequenced (See Gregory, R. J. et al. (1990) Nature 347:382-386; Rich, D. P. et al. (1990) Nature 347:358-362), (Riordan, J. R. et al. (1989) Science 245:1066-1073). CFTR, a member of the ATP binding cassette (ABC) superfamily is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate, and thiocyanate into and out of the cell. CFTR may have a regulatory role over other electrolyte channels, including the epithelial sodium channel ENaC.

In cystic fibrosis patients, the absence or dysfunction of CFTR leads to exocrine gland dysfunction and a multisystem disease, characterized by pancreatic insufficiency and malabsorption, as well as abnormal mucociliary clearance in the lung, mucostasis, chronic lung infection and inflammation, decreased lung function and ultimately respiratory failure.

While more than 1,900 mutations have been identified in the CFTR gene, a detailed understanding of how each CFTR mutation may impact channel function is known for only a few. (Derichs, European Respiratory Review, 22:127, 58-65 (2013)). The most frequent CFTR mutation is the in-frame deletion of phenylalanine at residue 508 (ΔF508) in the first nucleotide binding domain (NBD1). Over 70% of cystic fibrosis patients have a deletion at residue 508 in at least one CFTR allele. The loss of this key phenylalanine renders NBD1 conformationally unstable at physiological temperature and compromises the integrity of the interdomain interface between NDB1 and CFTR's second transmembrane domain (ICL4). The ΔF508 mutation causes production of misfolded CFTR protein which, rather than traffic to the plasma membrane, is instead retained in the endoplasmic reticulum and targeted for degradation by the ubiquitin-proteasome system.

The loss of a functional CFTR channel at the plasma membrane disrupts ionic homeostasis and airway surface hydration leading to reduced lung function. Reduced periciliary liquid volume and increased mucus viscosity impede mucociliary clearance resulting in chronic infection and inflammation. In the lung, the loss of CFTR-function leads to numerous physiological effects downstream of altered anion conductance that result in the dysfunction of additional organs such as the pancreas, intestine and gall bladder.

By studying the mechanistic aspects of CFTR misfolding and corrections, small molecules have been identified as CF modulators, that can act as stabilizers.

Despite the identification of compounds that modulate CFTR, there is no cure for this fatal disease and identification of new compounds and new methods of therapy are needed as well as new methods for treating or lessening the severity of cystic fibrosis and other CFTR mediated conditions and diseases in a patient.

### SUMMARY

The present disclosure includes a compound of formula I:

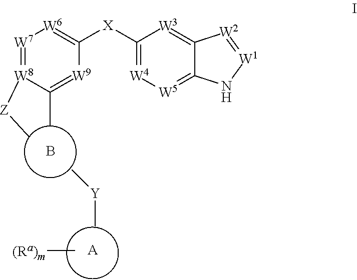

or a pharmaceutically acceptable salt thereof. Additionally, the present disclosure includes, among other things, pharmaceutical compositions, methods of using and methods of making a compound of formula I.

### DETAILED DESCRIPTION

In some embodiments, the present disclosure includes a compound of Formula I:

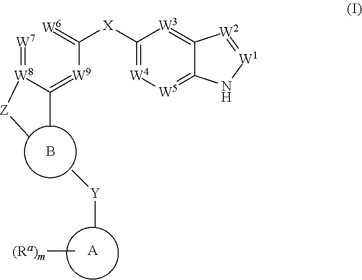

or a pharmaceutically acceptable salt thereof
   wherein
W¹ is selected from the group consisting of —C(H)═, and —N═;
W² is selected from the group consisting of —C(H)═, —C(R^{d})═, and —N═;
W³ is selected from the group consisting of —C(H)═, —C(R^{d})═, and —N═;
W⁴ is selected from the group consisting of —C(H)═, —C(R^{d4})═, and —N═;
W⁵ is selected from the group consisting of —C(H)═, —C(R^{d5})═, and —N═;
W⁶ is selected from the group consisting of —C(H)═, —C(R^{c})═, and —N═;
W⁷ is selected from the group consisting of —C(H)═, —C(R^{c})═, and —N═;
W⁸ is selected from the group consisting of C or N;
W⁹ is selected from the group consisting of —C(H)═, —C(R^{c9})═, and —N═;
Ring A is optionally substituted phenyl or optionally substituted 5-6-membered heteroaryl;
Ring B is optionally substituted 5-member heteroaryl, wherein R^{b} is optionally substituted with n instances of R^{b}.
each R^{a} is independently selected from the group consisting of halogen, oxo —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R^{J})₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa},
each R^{aa} is independently selected from the group consisting of halogen, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —SO₂R¹, —SO(NR²)R¹, —SO₂N(R²)R¹, —N(H)C(O)R¹, and —N(H)C(O)N(R¹)₂,
   wherein two instances of R^{a} are optionally taken together with any intervening atoms to form an optionally substituted 4-6 membered heterocyclyl ring;
each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{c} is independently selected from the group consisting of halogen, oxo —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
R^{c9} is halogen;
R^{d4} is halogen;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
   wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
X is selected from the group consisting of —O—, —S—, —S(O)—, —S(O)₂—, —SO(NR²)—, —N(R¹)—, —C(R¹)(R²)—, —C(O)—, and —CH(OH)—;
Y is selected from the group consisting of optionally substituted C₁-C₃ alkylene, —O—, —S—, —S(O)—, —SO(NR²)—, and —S(O)₂—;
Z is optionally substituted C₁-C₄ alkylene, wherein one methylene unit is optionally replaced with —O—, —N(R¹)—, S—, —S(O)—, or —S(O)₂—;
n is 0, 1, or 2; and
m is 0, 1, 2, or 3.

### X

In some embodiments, X is selected from the group consisting of —O—, —S—, —S(O)—, —S(O)₂—, —SO(NR²)—, —N(R¹)—, —C(R¹)(R²)—, —C(O)—, and —CH(OH)—. In some embodiments, X is —O—. In some embodiments, X is —S—. In some embodiments, X is —S(O)—. In some embodiments, X is —S(O)₂—. In some embodiments, X is —CH₂—. In some embodiments, X is —C(H)(CH₃)—. In some embodiments, X is —C(O)—. In some embodiments, X is —CH(OH)—.

### Y

In some embodiments, Y is selected from the group consisting of optionally substituted C₁-C₃ alkylene, —O—, —S—, —S(O)—, —SO(NR²)—, and —S(O)₂—. In some embodiments, Y is —CH₂—. In some embodiments —O—. In some embodiments, Y is —S—. In some embodiments, Y is —S(O)—. In some embodiments, Y is —S(O)₂—. In some embodiments, Y is —SO₂N(R²)—. In some embodiments, Y is —S(O)₂—.

### Z

In some embodiments, Z is optionally substituted C₁-C₄ alkylene, wherein one methylene unit is optionally replaced with —O—, —N(R¹)—, S—, —S(O)—, or —S(O)₂—. In some embodiments, Z is optionally substituted C₁-C₄ alkylene. In some embodiments, Z is —CH₂—. In some embodiments, Z is —CH₂CH₂—. In some embodiments, Z is —CH₂CH₂CH₂—. In some embodiments, Z is optionally substituted C₁-C₄ alkylene, wherein one methylene unit is replaced with —O—. In some embodiments, Z is —CH₂CH₂O—. In some embodiments, Z is optionally substituted C₃-C₄ alkenylene. In some embodiments, Z is —C(H)═C(H)—.

### Ring A

In some embodiments, Ring A is optionally substituted 5-membered heteroaryl, containing 1-2 heteroatoms selected from N, S, and O. In some embodiments, Ring A is an optionally substituted 5-membered heteroaryl selected from the group consisting of thiophenyl, pyrazolyl, pyrrolyl, and thiazolyl. In some embodiments, Ring A is optionally substituted thiophenyl. In some embodiments, Ring A is pyrazolyl. In some embodiments, Ring A is optionally substituted thiazolyl.

In some embodiments, Ring A is selected from the group consisting of

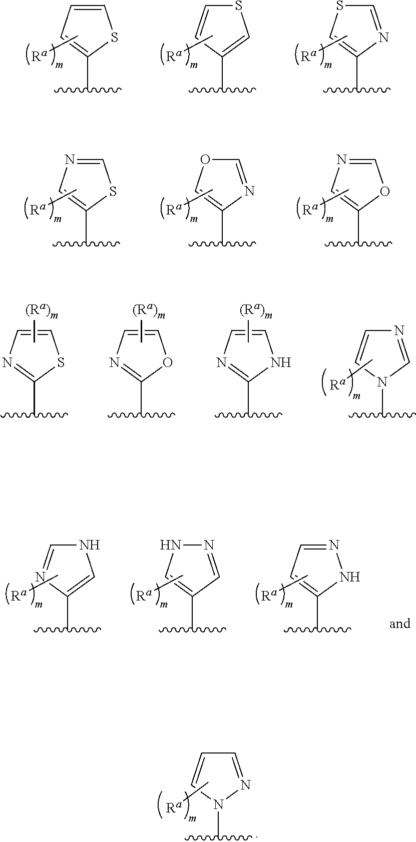

In some embodiments, Ring A is selected from the group consisting of

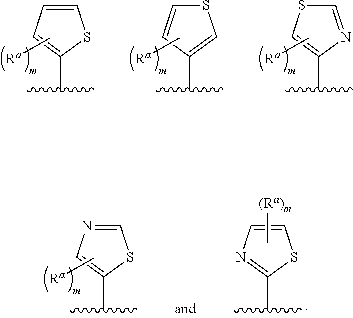

### Ring B

In some embodiments, Ring B is optionally substituted 5-membered heteroaryl. In some embodiments, Ring B is optionally substituted 5-membered heteroaryl comprising 1-3 nitrogen atoms. In some embodiments, Ring B is optionally substituted 5-member heteroaryl selected from the group consisting of pyrrolyl, pyrazolyl, imidazolyl, triazolyl, furanyl, oxazolyl, thiophenyl, and thiazolyl. In some embodiments, Ring B is a optionally substituted pyrazolyl. In some embodiments, Ring B is a optionally substituted triazolyl. In some embodiments, Ring B is a optionally substituted imidazolyl. In some embodiments, Ring B is a optionally substituted oxazolyl. In some embodiments, Ring B is a optionally substituted thiazolyl. In some embodiments, Ring B is a optionally substituted oxadiazolyl. In some embodiments, Ring B is a optionally substituted thiadiazolyl. In some embodiments, Ring B is a optionally substituted isooxadiazolyl. In some embodiments, Ring B is a optionally substituted isothiadiazolyl. In some embodiments, Ring B is selected from the group consisting of

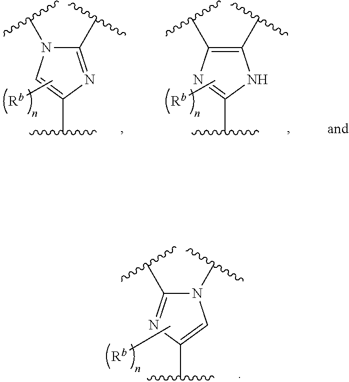

In some embodiments, Ring B is selected from the group consisting of

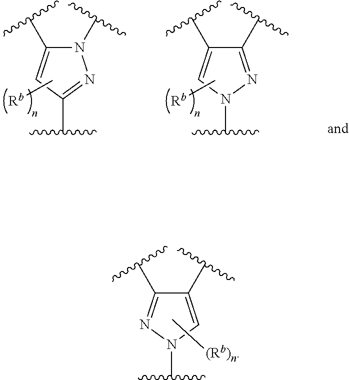

In some embodiments, Ring B is

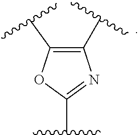 [SMILES: *c1nc(*)c(*)o1]

In some embodiments, Ring B is

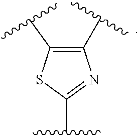 [SMILES: *c1nc(*)c(*)s1]

In some embodiments, Ring B is

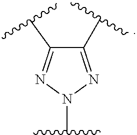 [SMILES: *c1nn(*)nc1*]

### R^{a}

In some embodiments, each R^{a} is selected from the group consisting of halogen, —CN, —NO₂—OR¹, —SR¹, —N(R¹)₂, —C(O)OR¹, C(O)N(R¹)₂, —N(H)C(O)R¹, —SO₂R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa}. In some embodiments, each R^{a} is independently selected from halogen, optionally substituted C₁-C₆ alkyl, and optionally substituted C₁-C₆ alkenyl, wherein each R^{a} is independently substituted with 0-4 instances of R^{aa}. In some embodiments, wherein each R^{a} is independently —CH₂COOH, —CH₂CH₂COOH, and —C(H)═C(H)—COOH. In some embodiments, wherein R^{a} is —CH₂COOH. In some embodiments, wherein R^{a} is —CH₂CH₂COOH. In some embodiments, wherein R^{a} is —C(H)═C(H)—COOH. In some embodiments, R^{a} is —CH₂CH(Me)CO₂H. In some embodiments, R^{a} is —CH₂CH(OH)CH₂(OH). In some embodiments, R^{a} is halogen. In some embodiments, R^{a} is chloro. In some embodiments, R^{a} is fluoro. In some embodiments, R^{a} is —CH₂COOR¹. In some embodiments, each R^{a} is —CH₂COOEt.

### R^{b}

In some embodiments, each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, R^{b} is optionally substituted C₁-C₆ aliphatic. In some embodiments, R^{b} is optionally substituted C₁-C₆ alkyl. In some embodiments, R^{b} is optionally substituted C₁-C₃ alkyl. In some embodiments, R^{b} is optionally substituted methyl. In some embodiments, two instances of R^{b} are taken together, with any intervening atoms to form a 5-7 members optionally substituted carboxylic or heteroaryl ring. In some embodiments, R^{b} is —N(R¹)₂. In some embodiments, R^{b} is —N(H)CH₂CH₂OH. In some embodiments, R^{b} is piperidonyl. In some embodiments, R^{b} is pyrrolidinonyl.

### R^{c}

In some embodiments, each R^{c} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R³)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{c} is independently substituted with 0-4 instances of R^{aa}.

In some embodiments, R^{c} is halogen. In some embodiments, R^{c} is fluoro.

### R^{d}

In some embodiments, each R^{d} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO₂N(R²), —SO(NR²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{d} is independently substituted with 0-4 instances of R^{aa}. In some embodiments, R^{d} is independently selected from the group consisting of halogen, —OR¹, —SR¹, —C(O)N(R¹)₂, —N(H)C(O)R¹, —SO₂R¹, —SO₂N(R²), —SO(NR²)R¹, and optionally substituted C₁-C₆ aliphatic, wherein each R^{d} is independently substituted with 0-4 instances of R^{aa}. In some embodiments, each R^{d} is halogen. In some embodiments, R^{d} is fluoro.

In some embodiments, R^{d4} is halogen. In some embodiments, R^{d4} is fluoro.

In some embodiments, R^{d5} is halogen. In some embodiments, R^{d}s is fluoro.

In some embodiments, the present disclosure includes compounds listed in Table 1.

TABLE 1

| Compound No. | Structure |
|---|---|
| 1 | 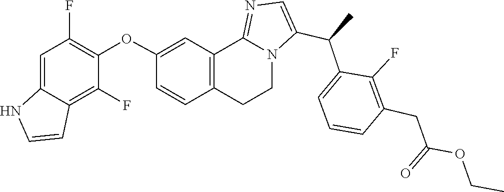 [SMILES: CCOC(=O)Cc1cccc([C@H](C)c2cnc3n2CCc2ccc(Oc4c(F)cc5[nH]ccc5c4F)cc2-3)c1F] |
| 2 | 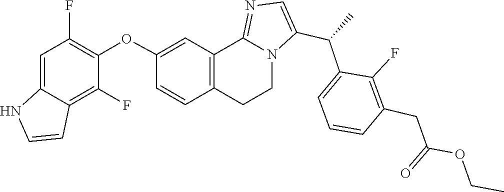 [SMILES: CCOC(=O)Cc1cccc([C@@H](C)c2cnc3n2CCc2ccc(Oc4c(F)cc5[nH]ccc5c4F)cc2-3)c1F] |
| 3 | 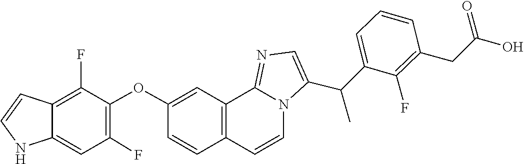 [SMILES: CC(c1cccc(CC(=O)O)c1F)c1cnc2c3cc(Oc4c(F)cc5[nH]ccc5c4F)ccc3ccn12] |
| 4 | 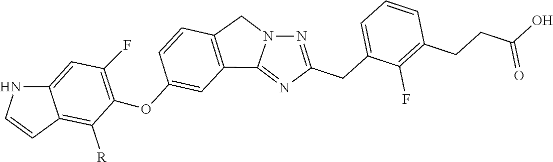 |
| 5 | 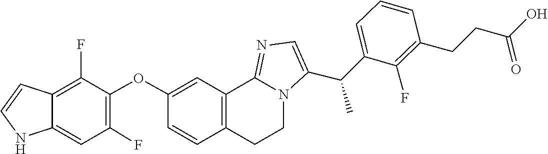 [SMILES: C[C@@H](c1cccc(CCC(=O)O)c1F)c1cnc2n1CCc1ccc(Oc3c(F)cc4[nH]ccc4c3F)cc1-2] |
| 6 | 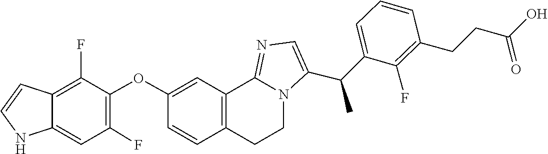 [SMILES: C[C@H](c1cccc(CCC(=O)O)c1F)c1cnc2n1CCc1ccc(Oc3c(F)cc4[nH]ccc4c3F)cc1-2] |
| 7 | 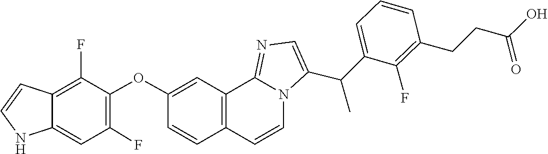 [SMILES: CC(c1cccc(CCC(=O)O)c1F)c1cnc2c3cc(Oc4c(F)cc5[nH]ccc5c4F)ccc3ccn12] |
| 8 | 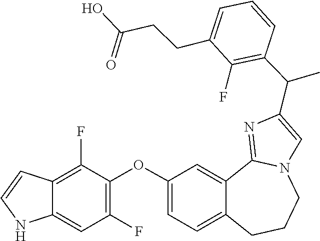 [SMILES: CC(c1cn2c(n1)-c1cc(Oc3c(F)cc4[nH]ccc4c3F)ccc1CCC2)c1cccc(CCC(=O)O)c1F] |
| 9 | 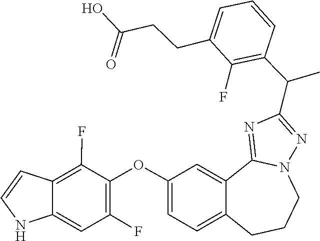 [SMILES: CC(c1nc2n(n1)CCCc1ccc(Oc3c(F)cc4[nH]ccc4c3F)cc1-2)c1cccc(CCC(=O)O)c1F] |
| 10 | 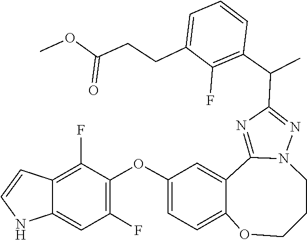 [SMILES: COC(=O)CCc1cccc(C(C)c2nc3n(n2)CCCOc2ccc(Oc4c(F)cc5[nH]ccc5c4F)cc2-3)c1F] |
| 11 | 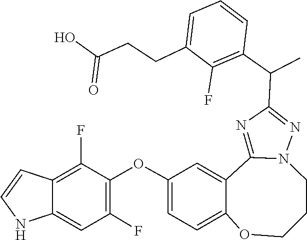 [SMILES: CC(c1nc2n(n1)CCCOc1ccc(Oc3c(F)cc4[nH]ccc4c3F)cc1-2)c1cccc(CCC(=O)O)c1F] |
| 12 | 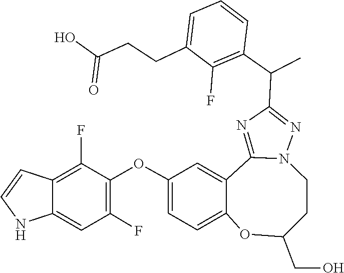 [SMILES: CC(c1nc2n(n1)CCC(CO)Oc1ccc(Oc3c(F)cc4[nH]ccc4c3F)cc1-2)c1cccc(CCC(=O)O)c1F] |
| 13 | 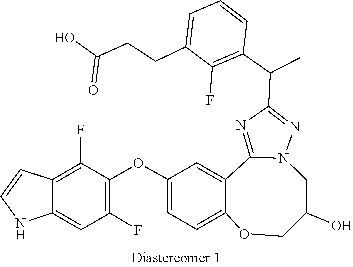 [SMILES: CC(c1nc2n(n1)CC(O)COc1ccc(Oc3c(F)cc4[nH]ccc4c3F)cc1-2)c1cccc(CCC(=O)O)c1F] |
| 14 | 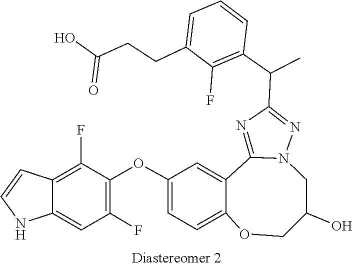 [SMILES: CC(c1nc2n(n1)CC(O)COc1ccc(Oc3c(F)cc4[nH]ccc4c3F)cc1-2)c1cccc(CCC(=O)O)c1F] |
| 15 | 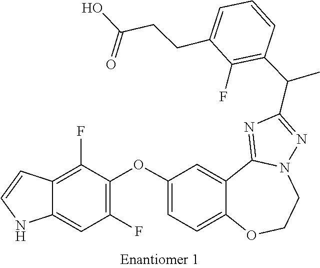 [SMILES: CC(c1nc2n(n1)CCOc1ccc(Oc3c(F)cc4[nH]ccc4c3F)cc1-2)c1cccc(CCC(=O)O)c1F] |
| 16 | 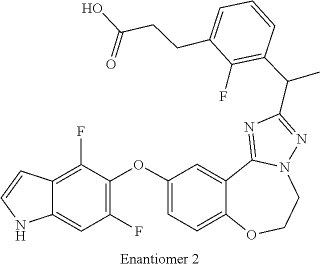 [SMILES: CC(c1nc2n(n1)CCOc1ccc(Oc3c(F)cc4[nH]ccc4c3F)cc1-2)c1cccc(CCC(=O)O)c1F] |

or a pharmaceutically acceptable salt thereof.

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “haloalkyl” refers to a straight or branched alkyl group that is substituted with one or more halogen atoms.

The term “alkyl” as used herein is a branched or unbranched saturated hydrocarbon group having a specified number of carbon atoms. In some embodiments, alkyl refers to a branched or unbranched saturated hydrocarbon group having three carbon atoms (C₃). In some embodiments, alkyl refers to a branched or unbranched saturated hydrocarbon group having six carbon atoms (C₆). In some embodiments, the term “alkyl” includes, but is not limited to, methyl, ethyl, n-propyl, isopropyl, n-butyl, isobutyl, s-butyl, t-butyl, n-pentyl, isopentyl, s-pentyl, neopentyl, and hexyl.

As used herein, the term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group. The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the disclosure may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —S(O)(NR^{∘})R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{c}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{c} (or the ring formed by taking two independent occurrences of R^{c} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat and/or diagnose the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, reduces severity of and/or reduces incidence of one or more symptoms or features of the disease, disorder, and/or condition. In some embodiments, a “therapeutically effective amount” is at least a minimal amount of a provided compound, or composition containing a provided compound, which is sufficient for treating one or more symptoms of an CFTR-associated disease or disorder.

The terms “treat”, “treatment” or “treating” mean to decrease, suppress, attenuate, diminish, arrest, or stabilize the development or progression of a disease (e.g., a disease or disorder delineated herein), lessen the severity of the disease or improve the symptoms associated with the disease. Treatment includes treating a symptom of a disease, disorder or condition. Without being bound by any theory, in some embodiments, treating includes augmenting deficient CFTR activity. If it is administered prior to clinical manifestation of the unwanted condition (e.g., disease or other unwanted state of the subject) then the treatment is prophylactic (i.e., it protects the subject against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

A “response” to a method of treatment can include a decrease in or amelioration of negative symptoms, a decrease in the progression of a disease or symptoms thereof, an increase in beneficial symptoms or clinical outcomes, a lessening of side effects, stabilization of disease, partial or complete remedy of disease, among others.

As used herein, “CFTR” means cystic fibrosis transmembrane conductance regulator. Defects in the function of the CFTR ion channel result from loss of function mutations of CFTR. Such mutations lead to exocrine gland dysfunction, abnormal mucociliary clearance, and cause cystic fibrosis. The most common CFTR mutation in Cystic Fibrosis (CF) patients leads to the specific deletion of three nucleotides of the codon for phenylalanine at position 508. This mutation, which is found in ~70% of CF patients worldwide, is referred to as “ΔF508”. The ΔF508 mutation decreases the stability of the CFTR NBD1 domain and limits CFTR interdomain assembly. Since CF is an autosomal recessive disease, a CF patient harboring the ΔF508 CFTR mutation must also carry a second defective copy of CFTR. Approximately 2000 different CF-causing CFTR mutations have been identified in CF patients. CF patients harboring the ΔF508 CFTR mutation can be homozygous for that mutation (ΔF508/ΔF508). CF patients can also be ΔF508 heterozygous, if the second CFTR allele such patients carry instead contains a different CFTR loss of function mutation. Such CFTR mutations include, but are not limited to, G542X, G551D, N1303K, W1282X, R553X, R117H, R1162X, R347P, G85E, R560T, A455E, A1507, G178R, S549N, S549R, G551S, G970R, G1244E, 51251N, 51255P, and G1349D.

As used herein, the term “CFTR modulator” refers to a compound that increases the activity of CFTR. In certain aspects, a CFTR modulator is a CFTR corrector or a CFTR potentiator or a dual-acting compound having activities of a corrector and a potentiator.

As used herein, the term “CFTR corrector” refers to a compound that increases the amount of functional CFTR protein to the cell surface and thus enhances CFTR channel function. The CFTR correctors partially “rescue” misfolding of CFTR, thereby enabling the maturation and functional expression of CFTR protein harboring a CF causing mutation on the cell surface. Examples of correctors include, but are not limited to, VX-809, VX-661, VX-152, VX-440, VX-983, and GLPG2222. Such compounds may interact directly with CFTR protein, modifying its folding and conformational maturation during synthesis.

As used herein, the term “CFTR potentiator” refers to a compound that increases the ion channel activity of CFTR protein located at the cell surface, resulting in enhanced ion transport. CFTR potentiators repair the defective channel functions caused by mutations. Examples of potentiators include, but are not limited to, ivacaftor (VX770), deuterated ivacaftor (CPT 656), genistein and GLPG1837.

As used herein, the term “CFTR pharmacological chaperone” (PC) refers to compounds that stabilize the CFTR protein in its native state by binding directly to the protein.

As used herein, the term “CFTR proteostasis regulator” (PR) refers to compounds that enhance the protein folding efficiency within the cell. PRs can alter the activity of transcriptional, folding and/or membrane trafficking machinery, as well as impeding the degradation of partially folded, but functional, conformers at the endoplasmic reticulum (ER) or plasma membrane.

As used herein, “CFTR disease or condition” refers to a disease or condition associated with deficient CFTR activity, for example, cystic fibrosis, congenital bilateral absence of vas deferens (CBAVD), acute, recurrent, or chronic pancreatitis, disseminated bronchiectasis, asthma, allergic pulmonary aspergillosis, smoking-related lung diseases, such as chronic obstructive pulmonary disease (COPD), chronic sinusitis, dry eye disease, protein C deficiency, A-beta.-lipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a provided compound, an additional therapeutic agent, and a pharmaceutically acceptable carrier, adjuvant, or vehicle.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of the present disclosure and a pharmaceutically acceptable carrier, adjuvant, or vehicle. In some embodiments, the amount of compound in compositions contemplated herein is such that is effective to measurably modulate CFTR, or a mutant thereof, in a biological sample or in a patient. In certain embodiments, the amount of compound in compositions of this disclosure is such that is effective to measurably modulate CFTR, or a mutant thereof, in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, the amount of compound in compositions contemplated herein is such that is effective to measurably modulate a protein, particularly at CFTR, or a mutant thereof, in a biological sample or in a patient. In certain embodiments, the amount of compound in compositions of this disclosure is such that is effective to measurably modulate CFTR, or a mutant thereof, in a biological sample or in a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of the present disclosure may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium.

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of the present disclosure may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of the present disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of the present disclosure may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of the present disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

As discussed above, CFTR is composed of two six membrane-spanning domains (MSD1 and MSD2), two nucleotide bind domains (NBD1 and NBD2), a regulatory region (R) and four cytosolic loops (CL1-4). CFTR protein is located primarily in the apical membrane of epithelial cells where it functions to conduct anions, including chloride, bicarbonate and thiocyanate into and out of the cell. The most frequent CFTR mutation is the in-frame deletion of phenylalanine at residue 508 (ΔF508) in the first nucleotide binding domain (NBD1). The mutation has several deleterious effects on the production of CFTR in the ER, its correct folding, its movement to the plasma membrane and its normal function as an ion channel for the cell.

One such negative effect is that the NBD1 domain is partially or mis-folded which is recognized within the cell as an aberrant protein and tagged for disposal by ER-associated degradation (ERAD) via the ubiquitin-proteasome system (UPS). Should a partially or mis-folded CFTR protein emerge from the ER, the protein must travel to the plasma membrane through complex glycosylation in the Golgi compartment and be functionally inserted. In wild-type CFTR, only 20-40% of CFTR reaches the plasma membrane, indicating that CFTR has energetic instability of individual NBDs, a slow domain assembly, and relatively fast ERAD kinetics which all contribute to inefficient folding and sensitize CFTR to structural perturbations by mutations.

In wild-type CFTR, the NBD1 domain folds co-translationally while other domains fold post-translationally. Mutated ΔF508 CFTR has impaired NBD1 folding but its backbone structure and thermodynamic stability are similar to wild-type CFTR. With delayed folding kinetics, mutated ΔF508 CFTR NBD1 has an increased folding activation energy. Lack of proper folding results in hydrophobic residues being exposed to the surface of NBD1 which causes aggregation with other CFTR proteins. Thus, the aggregation temperature of mutated CFTR drops from 41° C. to 33° C. This level of instability creates a greater percentage of mis-folded mutant CFTR at physiological temperature (37° C. in humans). Mutant CFTR suffers from both kinetic and thermodynamic folding defects. CFTR stabilizers can address these folding defects, but complete energetic correction of mutant NBD1 folding has been shown to not result in the CFTR biosynthetic processing, underscoring the need for interface stability as well.

The disclosed CFTR correctors can interact with the NBD domain to stabilize the correct folded position R, such that CFTR is not labeled for elimination from the cell. The preservation of correct folding enables CFTR to function as a chloride ion channel at wild-type levels. In some embodiments, disclosed CFTR correctors can enhance the performance of wild-type CFTR.

CFTR stabilizers can function in combination with other therapeutic agents such as CFTR correctors that promote A508 CFTR exit from the ER and accumulation in the plasma membrane. Increasing the amount of CFTR cell surface expression can result in improved chloride conductance following channel activation by both potentiators and a cAMP agonist. Thus, disclosed herein are combinations of CFTR stabilizers with CFTR correctors and potentiators, optionally with cAMP agonists or another therapeutic agent as described below.

Disclosed herein are methods of treating deficient CFTR activity in a cell, comprising contacting the cell with a compound of the present disclosure, or a pharmaceutically acceptable salt thereof. In certain embodiments, contacting the cell occurs in a subject in need thereof, thereby treating a disease or disorder mediated by deficient CFTR activity.

Also, disclosed herein are methods of treating a disease or a disorder mediated by deficient CFTR activity comprising administering a compound of the present disclosure or a pharmaceutically acceptable salt thereof. In some embodiments, the subject is a mammal, preferably a human. In some embodiments, the disease is associated with the regulation of fluid volumes across epithelial membranes, particularly an obstructive airway disease such as CF or COPD.

Such diseases and conditions include, but are not limited to, cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

Such diseases and conditions include, but are not limited to, cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome. In some embodiments, a disease is cystic fibrosis.

Provided herein are methods of treating cystic fibrosis, comprising administering to a subject in need thereof, a compound as disclosed herein or a pharmaceutically acceptable salt thereof. Also provided herein are methods of lessening the severity of cystic fibrosis, comprising administering to a subject in need thereof, a compound as disclosed herein or a pharmaceutically acceptable salt thereof. In some embodiments, the subject is a human. In some embodiments, the subject is at risk of developing cystic fibrosis, and administration is carried out prior to the onset of symptoms of cystic fibrosis in the subject.

Provided herein are compounds as disclosed herein for use in treating a disease or condition mediated by deficient CFTR activity. Also provided herein are uses of a compound as disclosed herein for the manufacture of a medicament for treating a disease or condition mediated by deficient CFTR activity.

Provided herein are kits for use in measuring the activity of CFTR or a fragment thereof in a biological sample in vitro or in vivo. The kit can contain: (i) a compound as disclosed herein, or a pharmaceutical composition comprising the disclosed compound, and (ii) instructions for: a) contacting the compound or composition with the biological sample; and b) measuring activity of said CFTR or a fragment thereof. In some embodiments, the biological sample is biopsied material obtained from a mammal or extracts thereof; blood, saliva, urine, feces, semen, tears, other body fluids, or extracts thereof. In some embodiments, the mammal is a human.

Provided herein are compounds as disclosed herein for use in treating kidney disease. In some embodiments, a kidney disease is autosomal dominant polycystic kidney disease (ADPKD) or autosomal recessive polycystic kidney disease (ARPKD). In some embodiments, a kidney disease is autosomal dominant polycystic kidney disease (ADPKD). In some embodiments, a kidney disease is autosomal recessive polycystic kidney disease (ARPKD).

### Combination Treatments

As used herein, the term “combination therapy” means administering to a subject (e.g., human) two or more CFTR modulators, or a CFTR modulator and an agent such as antibiotics, ENaC inhibitors, GSNO (S-nitrosothiol, s-nitroglutathione) reductase inhibitors, and a CRISPR Cas correction therapy or system (as described in US 2007/0022507 and the like). In some embodiments, combination therapy includes administration of a compound described herein with a compound that modulates CFTR protein or ABC protein activities (e.g., as described in WO2018167690A1 and the like)

In certain embodiments, the method of treating a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

In certain embodiments, the method of preventing a disease or condition mediated by deficient CFTR activity comprises administering a compound as disclosed herein conjointly with one or more other therapeutic agent(s). In some embodiments, one other therapeutic agent is administered. In other embodiments, at least two other therapeutic agents are administered.

Additional therapeutic agents include, for example, ENaC inhibitors, mucolytic agents, modulators of mucus rheology, bronchodilators, antibiotics, anti-infective agents, anti-inflammatory agents, ion channel modulating agents, therapeutic agents used in gene or mRNA therapy, agents that reduce airway surface liquid and/or reduce airway surface PH, CFTR correctors, and CFTR potentiators, or other agents that modulate CFTR activity. Other therapeutics include liposomal composition components such as those described in WO2012/170889, hybrid oligonucleotides that facilitate RNA cleavage such as those described in WO2016/130943, and single stranded oligonucleotides that modulate gene expression as described in WO2016/130929.

In some embodiments, at least one additional therapeutic agent is selected from one or more CFTR modulators, one or more CFTR correctors and one or more CFTR potentiators.

Non-limiting examples of additional therapeutics include VX-770 (Ivacaftor), VX-809 (Lumacaftor, 3-(6-(I-(2,2-5 difluorobenzo[d][1, 3]dioxol-5-yl)cyclopropanecarboxamido)-3-methylpyridin-2-yl) benzoic acid, VX-661 (Tezacaftor, I-(2,2-difluoro-1, 3-benzodioxol-5-yl)-N—[I-[(2R)-2,3-dihydroxypropyl]-6-fluoro-2-(2-hydroxy-1, I-dimethylethyl)-IH-indol-5-yl]-cyclopropanecarboxamide), VX-983, VX-152, VX-440, VX-445, VX-659, VX-371, Orkambi, Ataluren (PTC 124) (3-[5-(2-fluorophenyl)-1, 2,4-oxadiazol-3-yl]benzoic acid), PTI-130 (Proteostasis), PTI-801, PTI-808, PTI-428, N91115.74 (cavosonstat), QBW251 (Novartis) compounds described in WO2011113894, compounds N30 Pharmaceuticals (e.g., WO 2014/186704), deuterated ivacaftor (e.g., CTP-656 or VX-561), GLPG 2222, GLPG2451, GLPG3067, GLPG2851, GLPG2737, GLPG 1837 (N-(3-carbamoyl-5,5,7,7-tetramethyl-5,7-dihydro-4H-thieno[2,3-c]pyran-2-yl)-1H-pyrazole-5-carboxamide), GLPG 2665 (Galapagos), FDL 169 (Flatley Discovery lab), FDL 176, FDL438, FDL304, FD2052160, FD1881042, FD2027304, FD2035659, FD2033129, FD1860293, CFFT-Pot01, CFFT-Pot-02, P-1037, glycerol, phenylbutyrate, and the like.

Non-limiting examples of additional therapeutics include compounds disclosed in US Patent Application Nos. PCT/US20/63586, PCT/US20/63589, and PCT/US20/63590, each of which is incorporated by reference in its entirety.

Non-limiting examples of anti-inflammatory agents are N6022 (3-(5-(4-(1H-imidazol-1-yl)₁₀ phenyl)-1-(4-carbamoyl-2-methylphenyl)-′H-pyrrol-2-yl) propanoic acid), Ibuprofen, Lenabasum (anabasum), Acebilustat (CTX-4430), LAU-7b, POL6014, docosahexaenoic acid, alpha-1 anti-trypsin, sildenafil. Additional therapeutic agents also include, but are not limited to a mucolytic agent, a modifier of mucus rheology (such as hypertonic saline, mannitol, and oligosaccharide based therapy), a bronchodilator, an anti-infective (such as tazobactam, piperacillin, rifampin, meropenem, ceftazidime, aztreonam, tobramycin, fosfomycin, azithromycin, amitriptyline, vancomycin, gallium and colistin), an anti-infective agent, an anti-inflammatory agent, a CFTR modulator other than a compound of the present disclosure, and a nutritional agent. Additional therapeutic agents can include treatments for comorbid conditions of cystic fibrosis, such as exocrine pancreatic insufficiency which can be treated with Pancrelipase or Liprotamase.

Examples of CFTR potentiators include, but are not limited to, Ivacaftor (VX-770), CTP-656, NVS-QBW251, FD1860293, GLPG2451, GLPG1837, and N-(3-carbamoyl-5,5,7,7-tetramethyl-5,7-dihydro-4H-thieno[2,3-c]pyran-2-yl)-1H-pyrazole-5-carboxamide. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014180562, WO2015018823, and U.S. patent application Ser. Nos. 14/271,080, 14/451,619 and 15/164,317.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661), VX-983, GLPG2222, GLPG2665, GLPG2737, VX-152, VX-440, FDL169, FDL304, FD2052160, and FD2035659. Examples of correctors are also disclosed in US20160095858A1, and U.S. application Ser. Nos. 14/925,649 and 14/926,727.

In certain embodiments, the additional therapeutic agent is a CFTR amplifier. CFTR amplifiers enhance the effect of known CFTR modulators, such as potentiators and correctors. Examples of CFTR amplifier include PTI130 and PTI-428. Examples of amplifiers are also disclosed in publications: WO2015138909 and WO2015138934.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, AZD5634, and VX-371. Additional agents that reduce the activity of the epithelial sodium channel blocker (ENaC) can be found, for example, in PCT Publication No. WO2009074575 and WO2013043720; and U.S. Pat. No. 8,999,976.

In one embodiment, the ENaC inhibitor is VX-371.

In one embodiment, the ENaC inhibitor is SPX-101 (S18).

In certain embodiments, the combination of a compound of the present disclosure, with a second therapeutic agent may have a synergistic effect in the treatment of cancer and other diseases or disorders mediated by adenosine. In other embodiments, the combination may have an additive effect.

### EXEMPLIFICATION

### Analytical Procedures

¹H NMR spectra were recorded with a Bruker AC 400 MHz apparatus. Chemical shifts (δ) are quoted in parts per million (ppm) and coupling constants (J) in hertz (Hz).

LC-MS spectra were obtained, unless noted otherwise, with a UPLC Acquity device of Waters for the liquid chromatography analysis, coupling with a ZMD (Waters) mass spectrometer. This system was piloted by MassLynx v4.1 software. Detection was made in UV at 220 nm.

Operational conditions for liquid chromatography analysis are as follows:
Column: Assentis Express C₁₈ 50×2.1 mm, 2.7 μm supelco
Eluent: Way A: H₂O+0.02% TFA;
   Way B: CH₃CN+0.014% TFA;
Gradient: T₀ min: 2% B, T₁ min: 98% B, T_{1.3} min: 98% B, T_{1.33} min: 2% B, T_{1.5} min: following injection;
Flow: 1 mL/min;
Temperature: 55° C.
   SQD: ESI+30V
   UV detection wavelength: 220 nm
   Injection volume: 0.2 μl.

Preparatory HPLC purification was carried out under the following conditions:
Instrument: Gilson 281 (PHG011)
Column Xtimate C18 21.2*250 mm, 10 μm
Mobile Phase: A: water (10 mM NH4HCO3 spiked with 0.025% NH3·H₂O); B: acetonitrile
Gradient: 5% B for 3 min, then 5-37% B in 10 min, stop at 18 min
Flow Rate: 30.00 mL/min
Detection Wavelength (nm): 214/254
Retention Time (min): 8

### Abbreviations

AcOH: acetic acid
AIBN: azobisisobutyronitrile
BINAP: 2,2′-bis(diphenylphosphino)-1,1′-binaphthyl
Boc: tert-butyloxycarbonyl
n-BuOH: n-butanol
DABAL-Me3: bis(trimethylaluminum)-1,4-diazabicyclo[2.2.2]octane adduct
DBU: 1,8-diazabicyclo[5.4.0]undec-7-ene
DCM: dichloromethane
DCE: 1,2-dichloroethane
DEA: diethyl amine
DIPEA: N,N-diisopropylethylamine
DMAP: 4-dimethylaminopyridine
DMF: N,N-dimethylformamide
DMF-DMA: N,N-dimethylformamide dimethyl acetal
DMSO: dimethyl sulfoxide
dppf: 1,1′-bis(diphenylphosphino)ferrocene
DTT: dithiothreitol
Ee: enantiomeric excess
Eq: equivalents
ESI: electron spray ionization
EtOAc: ethyl acetate
EtOH: ethanol
FA: formic acid
HATU: 1-[bis(dimethylamino)methylene]-1H-1,2,3-triazolo[4,5-b]pyridinium 3-oxide hexafluorophosphate
HPLC: high performance liquid chromatography
In vacuo: under vacuum; under reduced pressure
LAH: lithium aluminum hydride
LC-MS: liquid chromatography-mass spectrometry
LDA: lithium diisopropylamide
LHMDS: lithium bis(trimethylsilyl)amide
MeOH: methanol
NBS: N-bromosuccinimide
NIS: N-iodosuccinimide
NMP: N-methyl-2-pyrrolidone
Pd/C: Palladium on carbon
Prep-HPLC: preparative HPLC
SFC: supercritical fluid chromatography
TBAF: tetra-n-butylammonium fluoride
TBS: tert-butyldimethylsilyl
TFA: trifluoroacetic acid
TIPS: triisopropylsilyl
THE: tetrahydrofuran
THP: tetrahydropyran
TLC: thin layer chromatography
Ts: tosyl

### Example 1. Synthesis of (S)-2-(3-(1-(9-((4,6-difluoro-1H-indol-5-yl)oxy)-5,6-dihydroimidazo[2,1-a]isoquinolin-3-yl)ethyl)-2-fluorophenyl)acetic acid

### Example 2. Synthesis of (R)-2-(3-(1-(9-((4,6-difluoro-1H-indol-5-yl)oxy)-5,6-dihydroimidazo[2,1-a]isoquinolin-3-yl)ethyl)-2-fluorophenyl)acetic acid

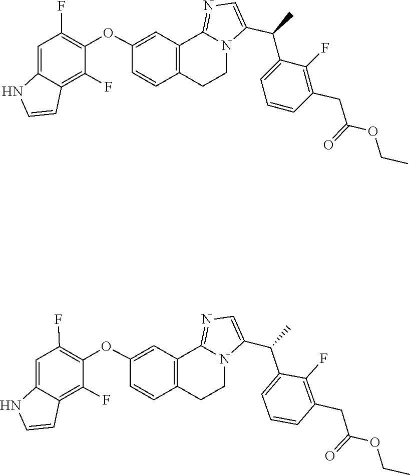 [SMILES: CCOC(=O)Cc1cccc([C@H](C)c2cnc3n2CCc2ccc(Oc4c(F)cc5[nH]ccc5c4F)cc2-3)c1F ; CCOC(=O)Cc1cccc([C@@H](C)c2cnc3n2CCc2ccc(Oc4c(F)cc5[nH]ccc5c4F)cc2-3)c1F]

### Methyl (4-methoxyphenethyl)carbamate

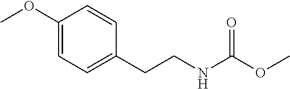

Step A: A mixture of 2-(4-methoxyphenyl)ethanamine (20.0 g, 132 mmol) and triethylamine (40.2 g, 397 mmol) in DCM (200 ml) was added dropwise to methyl carbonochloridate (13.1 g, 140 mmol). After addition, the mixture was stirred for another 2 hours. The mixture was then washed with saturated aqueous NaHCO₃ solution, brine, dried over anhydrous sodium sulfate and concentrated. The crude material was purified by flash column chromatography on silica, eluting with 10/1 petroleum ether/EtOAc, to afford methyl N-[2-(4-methoxyphenyl)ethyl]carbamate (20.0 g, 48%). MS (ESI): 210.2 m/z (M+H)⁺.

### 7-Hydroxy-3,4-dihydroisoquinolin-1(2H)-one

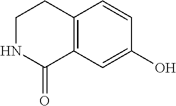 [SMILES: O=C1NCCc2ccc(O)cc21]

Step B: To methyl N-[2-(4-methoxyphenyl)ethyl]carbamate (17.0 g, 54.1 mmol) was added slowly at 0° C. trifluoromethanesulfonic acid (100 mL). The mixture was stirred at 70° C. for 24 hours, then cooled down, poured into ice-water (50 mL), and extracted with dichloromethane (50 mL×3). The combined organic phase was washed with brine, dried over Na₂SO₄, and concentrated in vacuo. The residue was purified by column chromatography on silica gel, eluting with 1:1 EtOAc:hexane, to afford 7-hydroxy-3,4-dihydro-2H-isoquinolin-1-one (12.5 g, 71%). MS (ESI): 164.1 m/z (M+H)⁺.

### 7-(2,6-Difluoro-4-nitrophenoxy)-3,4-dihydroisoquinolin-1(2H)-one

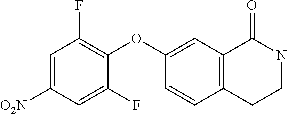 [SMILES: O=C1NCCc2ccc(Oc3c(F)cc([N+](=O)[O-])cc3F)cc21]

Step C: To a stirred solution of 1,2,3-trifluoro-5-nitrobenzene (6.81 g, 38.5 mmol) in DMF (50 mL) was added K₂CO₃ (10.7 g, 76.9 mmol) and 7-hydroxy-3,4-dihydro-2H-isoquinolin-1-one (12.0 g, 38.5 mmol). The resulting mixture was stirred at room temperature for 12 hours. The reaction was quenched with water (100 mL) and the mixture was extracted with EtOAc (100 mL×3). The combined organic layers were washed with saturated aqueous LiCl (30 mL), dried over Na₂SO₄ and concentrated. The residue was purified by silica gel column chromatography, eluting with 5:1 petroleum ether/EtOAc, to give 7-(2,6-difluoro-4-nitro-phenoxy)-3,4-dihydro-2H-isoquinolin-1-one (10.0 g, 77%) as a solid. MS (ESI): 321.1 m/z (M+H)⁺.

### 7-(4-Amino-2,6-difluorophenoxy)-3,4-dihydroisoquinolin-1(2H)-one

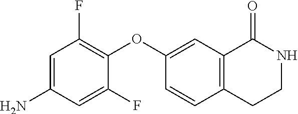 [SMILES: Nc1cc(F)c(Oc2ccc3c(c2)C(=O)NCC3)c(F)c1]

Step D: To a stirred suspension of 7-(2,6-difluoro-4-nitro-phenoxy)-3,4-dihydro-2H-isoquinolin-1-one (10.0 g, 29.7 mmol) in EtOH (150 mL) was added iron power (6.63 g, 119 mmol) and a solution of NH₄Cl (12.7 g, 237 mmol) in water (50 mL). The resulting mixture was heated at 80° C. and stirred for 4 hours. The insoluble material was removed by suction filtration and the filtrate was concentrated. The residue was dissolved in ethyl acetate (100 mL), washed with water (100 mL×3), brine (100 mL), dried over anhydrous Na₂SO₄, filtered, and concentrated to afford 7-(4-amino-2,6-difluoro-phenoxy)-3,4-dihydro-2H-isoquinolin-1-one (6.4 g, 61%) as a yellow solid. MS (ESI): 291.1 m/z (M+H)⁺.

### 7-(4-Amino-2,6-difluoro-3-iodophenoxy)-3,4-dihydroisoquinolin-1(2H)-one

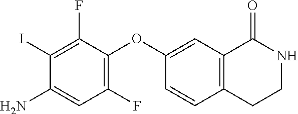 [SMILES: Nc1cc(F)c(Oc2ccc3c(c2)C(=O)NCC3)c(F)c1I]

Step E: To a stirred suspension of 7-(4-amino-2,6-difluoro-phenoxy)-3,4-dihydro-2H-isoquinolin-1-one (6.4 g, 18.2 mmol) in acetic acid (100 ml) was added N-iodosuccinimide (4.09 g, 18.2 mmol) portion wise at 0° C. The mixture was stirred at room temperature for one hour. The mixture was dissolved in ethyl acetate (200 ml) and washed with water (150 ml×3) and saturated aqueous NaHCO₃ (150 ml), dried over anhydrous Na₂SO₄, filtered, and concentrated in vacuo to afford the target compound as a yellow solid (6.8 g, 72%). MS (ESI): 417.0 m/z (M+H)⁺.

### 7-(4-Amino-2,6-difluoro-3-((trimethylsilyl)ethynyl)phenoxy)-3,4-dihydroisoquinolin-1(2H)-one

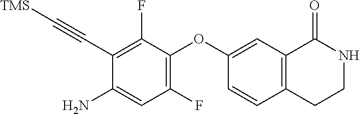 [SMILES: C[Si](C)(C)C#Cc1c(N)cc(F)c(Oc2ccc3c(c2)C(=O)NCC3)c1F]

Step F: To a stirred solution of 7-(4-amino-2,6-difluoro-3-iodo-phenoxy)-3,4-dihydro-2H-isoquinolin-1-one (6.8 g, 13.1 mmol) in DMF (80 ml) were added ethynyl(trimethyl)silane (3.87 g, 39.4 mmol), CuI (0.25 g, 1.31 mmol), triethylamine (3.98 g, 3.94 mmol) and Pd(dppf)Cl₂·DCM (1.07 g, 1.31 mmol). The reaction mixture was degassed with argon. The mixture was stirred overnight at 30° C. under an argon atmosphere and then quenched with water (200 mL). The mixture was extracted with ethyl acetate (3×100 ml). The organic layers were combined, washed with saturated aqueous LiCl solution (100 ml), dried over Na₂SO₄ and concentrated. The residue was chromatographed on silica gel, eluting with 3:1 petroleum ether:EtOAc, to provide 7-[4-amino-2,6-difluoro-3-(2-trimethylsilylethynyl)phenoxy]-3,4-dihydro-2H-isoquinolin-1-one (6.2 g, 81%) as a light brown solid. MS (ESI): 387.1 m/z (M+H)⁺.

### 7-((4,6-Difluoro-1H-indol-5-yl)oxy)-3,4-dihydroisoquinolin-1(2H)-one

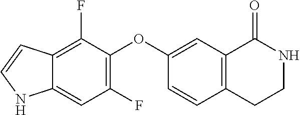 [SMILES: O=C1NCCc2ccc(Oc3c(F)cc4[nH]ccc4c3F)cc21]

Step G: To a stirred solution of 7-[4-amino-2,6-difluoro-3-(2-trimethylsilylethynyl)phenoxy]-3,4-dihydro-2H-isoquinolin-1-one (6.2 g, 10.5 mmol) in 1-methyl-2-pyrrolidinone (150 ml) was added potassium tert-butoxide (9.46 g, 84.6 mmol). The mixture was stirred at 80° C. for 2 hours. The solvent was removed under reduced pressure. The residue was purified by silica gel column chromatography, eluting with 1:1 petroleum ether:EtOAc, to give the desired product, 7-[(4,6-difluoro-1H-indol-5-yl)oxy]-3,4-dihydro-2H-isoquinolin-1-one (3.20 g, 59.9%) as a solid. MS (ESI): 315.1 m/z (M+H)⁺.

### 7-((4,6-Difluoro-1-tosyl-1H-indol-5-yl)oxy)-3,4-dihydroisoquinolin-1(2H)-one

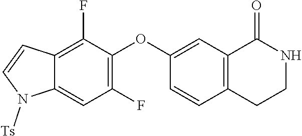 [SMILES: Cc1ccc(S(=O)(=O)n2ccc3c(F)c(Oc4ccc5c(c4)C(=O)NCC5)c(F)cc32)cc1]

Step H: To a mixture of 7-[(4,6-difluoro-1H-indol-5-yl)oxy]-3,4-dihydro-2H-isoquinolin-1-one (2.0 g, 3.94 mmol) and DBU (0.90 g, 5.92 mmol) in MeCN (20.0 ml) was added 1-(p-tolylsulfonyl)imidazole (0.96 g, 4.34 mmol). The mixture was stirred for 2 hours and concentrated. The crude material was purified by flash chromatography on silica gel, eluting with 2:1 petroleum ether:EtOAc, to afford 7-[4,6-difluoro-1-(p-tolylsulfonyl)indol-5-yl]oxy-3,4-dihydro-1H-isoquinolin-1-one (1.50 g, 77%). MS (ESI): 469.1 m/z (M+H)⁺.

### 7-((4,6-Difluoro-1-tosyl-1H-indol-5-yl)oxy)-3,4-dihydroisoquinolin-1(2H)-imine

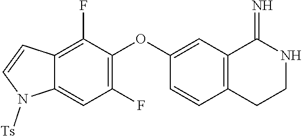 [SMILES: Cc1ccc(S(=O)(=O)n2ccc3c(F)c(Oc4ccc5c(c4)C(=N)NCC5)c(F)cc32)cc1]

Step I: To a stirred solution of 7-[4,6-difluoro-1-(p-tolylsulfonyl)indol-5-yl]oxy-3,4-dihydro-2H-isoquinolin-1-one (1.50 g, 3.04 mmol) in DCM (50 mL) was added Na₂CO₃ (1.61 g, 15.2 mmol) and Me₃OBF₄ (1.35 g, 9.12 mmol). The resulting mixture was stirred for 16 hours. Water (100 mL) was then added, and the mixture was extracted with EtOAc (30 mL×3). The combined organic phase was washed with brine (20 ml×3), dried over Na₂SO₄, filtered, and concentrated. The crude material was dissolved into 7N NH₃ in MeOH (5.0 ml), sealed and heated at 70° C. for 8 hours, then concentrated to give the desired product (1.0 g, 46%). MS (ESI): 468.1 m/z (M+H)⁺.

### Ethyl 2-(3-(1-(9-((4,6-difluoro-1H-indol-5-yl)oxy)-5,6-dihydroimidazo[2,1-a]isoquinolin-3-yl)ethyl)-2-fluorophenyl)acetate

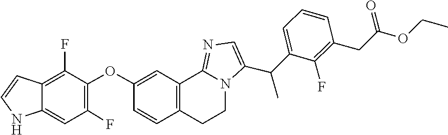 [SMILES: CCOC(=O)Cc1cccc(C(C)c2cnc3n2CCc2ccc(Oc4c(F)cc5[nH]ccc5c4F)cc2-3)c1F]

Step J: To a stirred solution of 7-[4,6-difluoro-1-(p-tolylsulfonyl)indol-5-yl]oxy-3,4-dihydro-2H-isoquinolin-1-imine (1.0 g, 1.39 mmol) in DMF (20 mL) was added Na₂CO₃ (0.73 g, 1.39 mmol) and ethyl 2-[3-(3-chloro-1-methyl-2-oxo-propyl)-2-fluoro-phenyl]acetate (0.399 g, 1.39 mmol). The resulting mixture was heated at 100° C. and stirred for 16 hours. After cooling to room temperature, water (100 mL) was added, and the mixture was extracted with EtOAc (30 mL×3). The combined organic phase was washed with a saturated aqueous LiCl solution (20 mL×3), dried over Na₂SO₄, filtered, and concentrated. The crude material was purified by flash column chromatography on silica to afford the desired product, ethyl 2-[3-[1-[9-[(4,6-difluoro-1H-indol-5-yl)oxy]-5,6-dihydroimidazo[2,1-a]isoquinolin-3-yl]ethyl]-2-fluorophenyl]acetate (0.50 g, 56%). MS (ESI): 546.2 m/z (M+H)⁺.

### Ethyl (R)-2-(3-(1-(9-((4,6-difluoro-1H-indol-5-yl)oxy)-5,6-dihydroimidazo[2,1-a]isoquinolin-3-yl)ethyl)-2-fluorophenyl)acetate and ethyl (S)-2-(3-(1-(9-((4,6-difluoro-1H-indol-5-yl)oxy)-5,6-dihydroimidazo[2,1-a]isoquinolin-3-yl)ethyl)-2-fluorophenyl)acetate

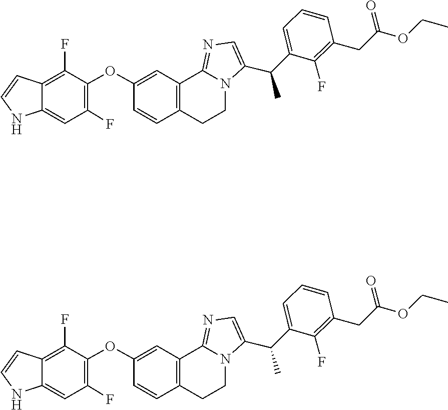 [SMILES: CCOC(=O)Cc1cccc([C@@H](C)c2cnc3n2CCc2ccc(Oc4c(F)cc5[nH]ccc5c4F)cc2-3)c1F ; CCOC(=O)Cc1cccc([C@H](C)c2cnc3n2CCc2ccc(Oc4c(F)cc5[nH]ccc5c4F)cc2-3)c1F]

Step K: Ethyl 2-[3-[1-[9-[(4,6-difluoro-1H-indol-5-yl)oxy]-5,6-dihydroimidazo[2,1-a]isoquinolin-3-yl]ethyl]-2-fluoro-phenyl]acetate (200 mg, 0.314 mmol) was separated by SFC into its constituent enantiomers. The absolute configuration of the faster eluting component was arbitrarily assigned as ethyl 2-[3-[(1R)-1-[9-[(4,6-difluoro-1H-indol-5-yl)oxy]-5,6-dihydroimidazo[2,1-a]isoquinolin-3-yl]ethyl]-2-fluoro-phenyl]acetate (68.0 mg), and the absolute configuration of the slower eluting enantiomer was correspondingly assigned as ethyl 2-[3-[(1S)-1-[9-[(4,6-difluoro-1H-indol-5-yl)oxy]-5,6-dihydroimidazo[2,1-a]isoquinolin-3-yl]ethyl]-2-fluorophenyl]acetate (68.0 mg).

### SFC Resolution Conditions:

Instrument: SFC-80 (Thar, Waters)
Column: SSWHELK 20*250 mm, 10 μm (Daicel)
Column temperature: 35° C.
Mobile phase: 30/70 CO₂/MeOH (0.2% Ammonia in Methanol)
Flow rate: 80 g/min; Back pressure: 100 bar
Detection wavelength: 214 nm; Cycle time: 4 min
Sample solution: 200 mg dissolved in 30 ml methanol; Injection volume: 2.5 ml

### (S)-2-(3-(1-(9-((4,6-Difluoro-1H-indol-5-yl)oxy)-5,6-dihydroimidazo[2,1-a]isoquinolin-3-yl)ethyl)-2-fluorophenyl)acetic acid

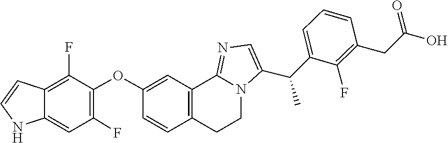 [SMILES: C[C@@H](c1cccc(CC(=O)O)c1F)c1cnc2n1CCc1ccc(Oc3c(F)cc4[nH]ccc4c3F)cc1-2]

Step L: To ethyl 2-[3-[(1S)-1-[9-[(4,6-difluoro-1H-indol-5-yl)oxy]-5,6-dihydroimidazo[2,1-a]isoquinolin-3-yl]ethyl]-2-fluoro-phenyl]acetate (68 mg, 0.112 mmol) was added LiOH·H₂O (23.5 mg, 0.56 mmol), water (1 ml) and MeOH (5 ml), and the reaction mixture was stirred at room temperature for 4 hours. After the starting material was consumed, the mixture was concentrated under reduced pressure and the residue was neutralized with 1N hydrochloric acid until pH-6. The mixture was then extracted with EtOAc (30 ml×2), the combined organic phase was washed with water, concentrated under reduced pressure, and purified by prep-HPLC to afford the desired compound, (S)-2-(3-(1-(9-((4,6-difluoro-1H-indol-5-yl)oxy)-5,6-dihydroimidazo[2,1-a]isoquinolin-3-yl)ethyl)-2-fluorophenyl)acetic acid (Example 1; 45.0 mg, 74%). MS (ESI): 518.2 m/z (M+H)⁺. ¹H NMR (400 MHz, DMSO-d₆): δ 11.66 (br, 1H), 8.29 (br, 1H), 7.47 (t, J=2.4 Hz, 1H), 7.32-7.27 (m, 2H), 7.15-7.11 (m, 3H), 7.03 (t, J=7.6 Hz, 1H), 6.95-6.87 (m, 2H), 6.56 (s, 1H), 4.26 (q, J=7.2 Hz, 1H), 4.10 (t, J=5.6 Hz, 2H), 3.56 (s, 2H), 3.04 (t, J=7.2 Hz, 2H), 1.47 (d, J=7.2 Hz, 3H) ppm.

### (R)-2-(3-(1-(9-((4,6-Difluoro-1H-indol-5-yl)oxy)-5,6-dihydroimidazo[2,1-a]isoquinolin-3-yl)ethyl)-2-fluorophenyl)acetic acid

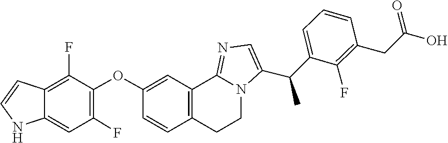 [SMILES: C[C@H](c1cccc(CC(=O)O)c1F)c1cnc2n1CCc1ccc(Oc3c(F)cc4[nH]ccc4c3F)cc1-2]

Step M: To ethyl 2-[3-[(1R)-1-[9-[(4,6-difluoro-1H-indol-5-yl)oxy]-5,6-dihydroimidazo[2,1-a]isoquinolin-3-yl]ethyl]-2-fluoro-phenyl]acetate (68 mg, 0.116 mmol) was added LiOH—H₂O (23.7 mg, 0.56 mmol), water (1 ml) and MeOH (5 ml), and the reaction mixture was stirred at room temperature for 4 hours. After the starting material was consumed, the mixture was concentrated, and the residue was neutralized with 1N hydrochloric acid until pH-6. The mixture was then extracted with EtOAc (30 ml×2). The combined organic phase was washed with water, brine, dried over sodium sulfate, filtered, and concentrated under reduced pressure. The crude material was purified by prep-HPLC to afford the desired compound (Example 2; 48.0 mg, 76%). MS (ESI): 518.2 m/z (M+H)⁺. ¹H NMR (400 MHz, DMSO-d₆): δ 11.66 (br, 1H), 8.29 (br, 1H), 7.47 (t, J=2.4 Hz, 1H), 7.32-7.27 (m, 2H), 7.15-7.11 (m, 3H), 7.03 (t, J=7.6 Hz, 1H), 6.95-6.87 (m, 2H), 6.56 (s, 1H), 4.26 (q, J=7.2 Hz, 1H), 4.10 (t, 2=5.6 Hz, 2H), 3.56 (s, 2H), 3.04 (t, J=7.2 Hz, 2H), 1.47 (d, J=7.2 Hz, 3H) ppm.

### Example 3. Synthesis of 2-(3-(1-(9-((4,6-difluoro-1H-indol-5-yl)oxy)imidazo[2,1-a]isoquinolin-3-yl)ethyl)-2-fluorophenyl)acetic acid

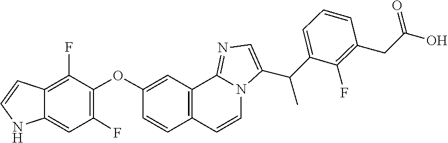 [SMILES: CC(c1cccc(CC(=O)O)c1F)c1cnc2c3cc(Oc4c(F)cc5[nH]ccc5c4F)ccc3ccn12]

### Ethyl 2-(3-(1-(9-((4,6-difluoro-1H-indol-5-yl)oxy)imidazo[2,1-a]isoquinolin-3-yl)ethyl)-2-fluorophenyl)acetate

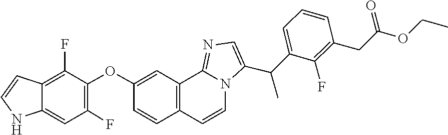 [SMILES: CCOC(=O)Cc1cccc(C(C)c2cnc3c4cc(Oc5c(F)cc6[nH]ccc6c5F)ccc4ccn23)c1F]

Step A: Pd/C (10 wt. %, 300 mg) was added to a solution of ethyl 2-[3-[1-[9-[(4,6-difluoro-1H-indol-5-yl)oxy]-5,6-dihydroimidazo[2,1-a]isoquinolin-3-yl]ethyl]-2-fluoro-phenyl]acetate (150 mg, 0.26 mmol) in Decalin (10 ml) under a nitrogen atmosphere. The mixture was stirred at 200° C. for 6 hours. After cooling to room temperature, the catalyst was removed by filtration and the filtrate was concentrated. This residue was purified by prep-HPLC to afford the desired product, ethyl 2-[3-[1-[9-[(4,6-difluoro-1H-indol-5-yl)oxy]imidazo[2,1-a]isoquinolin-3-yl]ethyl]-2-fluoro-phenyl]acetate (60 mg, 40%) as an oil. MS (ESI): 544.2 m/z (M+H)⁺.

### 2-(3-(1-(9-((4,6-Difluoro-1H-indol-5-yl)oxy)imidazo[2,1-a]isoquinolin-3-yl)ethyl)-2-fluorophenyl)acetic acid

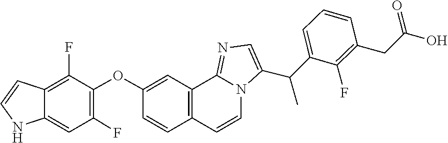 [SMILES: CC(c1cccc(CC(=O)O)c1F)c1cnc2c3cc(Oc4c(F)cc5[nH]ccc5c4F)ccc3ccn12]

Step B: To ethyl 2-[3-[1-[9-[(4,6-difluoro-1H-indol-5-yl)oxy]imidazo[2,1-a]isoquinolin-3-yl]ethyl]-2-fluoro-phenyl]acetate (60.0 mg, 0.10 mmol) was added LiOH·H₂O (30.9 mg, 0.75 mmol), water (1 ml) and MeOH (5 ml), and the reaction mixture was stirred at room temperature for 4 hours. After the starting material was consumed, the mixture was concentrated, and the residue was neutralized with 1N aqueous HCl until pH-6. The mixture was extracted with EtOAc (10 ml×2). The combined organic phase was washed with H₂O, brine, dried over sodium sulfate, filtered, and concentrated under reduced pressure. The residue was purified by prep-HPLC to afford 10.0 mg of the title compound (18%). MS (ESI): 516.2 m/z (M+H)⁺. ¹H NMR (400 MHz, DMSO-d₆): δ 11.76 (s, 1H), 8.36 (s, 1H), 8.21 (d, J=7.2 Hz, 1H), 7.89 (d, J=8.8 Hz, 1H), 7.64 (s, 1H), 7.58 (d, J=2.4 Hz, 1H), 7.50 (t, J=2.8 Hz, 1H), 7.41-7.36 (m, 2H), 7.20 (d, J=7.2 Hz, 1H), 7.17-7.06 (m, 2H), 7.01 (t, J=7.6 Hz, 1H), 6.59 (s, 1H), 4.47 (q, J=7.2 Hz, 1H), 3.50 (s, 2H), 1.56 (d, J=7.2 Hz, 3H) ppm.

### Example 4. Synthesis of 3-(3-((8-((4,6-difluoro-1H-indol-5-yl)oxy)-5H-triazolo[5,1-a]isoindol-2-yl)methyl)-2-fluorophenyl)propanoic acid

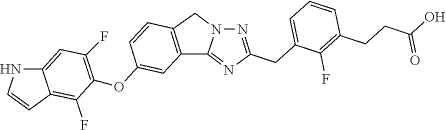 [SMILES: O=C(O)CCc1cccc(Cc2nc3n(n2)Cc2ccc(Oc4c(F)cc5[nH]ccc5c4F)cc2-3)c1F]

### 5-Hydroxy-2-methylbenzonitrile

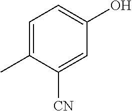

Step A: Concentrated sulfuric acid (50 mL, 940 mmol) was added to H₂O (100 mL). While the diluted acid was still hot, 5-amino-2-methylbenzonitrile (8.26 g, 62.5 mmol) was added giving a clear solution. This solution was cooled to 15° C. (during which time a precipitate formed) and 80 g of ice was added. As soon as the temperature dropped below 5° C., a solution of sodium nitrite (5.22 g, 75.7 mmol) in H₂O (50 mL) was added via syringe (with the needle extended below the surface of the liquid) keeping the internal temperature below 5° C. The solution went clear after 5 minutes of stirring, and then cold H₂O (50 mL), urea (590 mg, 9.7 mmol) and ice (50 g) were added sequentially. In a separate flask H₂O (50 mL) was added to sodium sulfate (47.5 g, 334 mmol) under an atmosphere of argon. Concentrated sulfuric acid (10 mL) was cautiously added and the reaction heated to reflux. The diazonium species was added to the refluxing mixture in portions and the heating continued for 2 hours (NOTE: Blast shield used during heating). The mixture was cooled to room temperature and extracted with EtOAc (200 mL×2). The combined organics were washed with brine (200 mL), dried over anhydrous sodium sulfate, filtered, and concentrated under reduced pressure. The residue was purified via flash column chromatography on silica, eluting with 0-30% acetone in petroleum ether, to afford the desired product, 5-hydroxy-2-methylbenzonitrile (5.47 g, 57%) as an orange solid. MS (ESI): 132.1 m/z (M−H)⁻.

### 5-(2,6-Difluoro-4-nitrophenoxy)-2-methylbenzonitrile

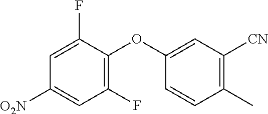 [SMILES: Cc1ccc(Oc2c(F)cc([N+](=O)[O-])cc2F)cc1C#N]

Step B: To a stirred solution of 5-hydroxy-2-methyl-benzonitrile (5.76 g, 39.8 mmol) and 1,2,3-trifluoro-5-nitrobenzene (7.05 g, 39.8 mmol) in DMF (70 mL) was added K₂CO₃ (11.0 g, 79.6 mmol). The resulting mixture was heated at 100° C. for 2 hours. After cooling to room temperature, the reaction mixture was added to water (50 mL) and the solid was collected by filtration. The solid was washed with water (50 mL) and dried to afford the desired product, 5-(2,6-difluoro-4-nitro-phenoxy)-2-methyl-benzonitrile (11.7 g, 88%) as a yellow solid. MS (ESI): 291.2 m/z (M+H)⁺.

### 4-(4-Amino-2,6-difluoro-phenoxy)-2-iodo-benzonitrile

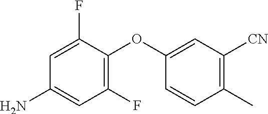 [SMILES: Cc1ccc(Oc2c(F)cc(N)cc2F)cc1C#N]

Step C: A mixture of 4-(2,6-difluoro-4-nitro-phenoxy)-2-iodo-benzonitrile (11.7 g, 25.3 mmol), Fe (5.66 g, 101 mmol) and NH₄Cl (10.8 g, 203 mmol) in EtOH (160 mL) and H₂O (40 mL) was heated refluxed under a nitrogen atmosphere for 16 hours. The reaction mixture was filtered while hot. The filtrate was evaporated under reduced pressure and the residue was dissolved in EtOAc (500 mL) and washed with brine (100 mL). The organic layer was dried over anhydrous sodium sulfate, filtered, and concentrated under reduced pressure to afford the desired product, 4-(4-amino-2,6-difluoro-phenoxy)-2-iodo-benzonitrile (10.6 g, 99%) as a yellow solid. MS (ESI): 261.2 m/z (M+H)⁺.

### 5-(4-Amino-2,6-difluoro-3-iodo-phenoxy)-2-methyl-benzonitrile

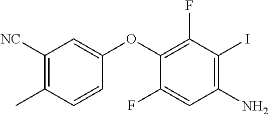 [SMILES: Cc1ccc(Oc2c(F)cc(N)c(I)c2F)cc1C#N]

Step D: To a stirred solution of 5-(4-amino-2,6-difluoro-phenoxy)-2-methyl-benzonitrile (10.6 g, 36.3 mmol) in acetic acid (100 mL) was added at room temperature in small portions N-iodosuccinimide (8.16 g, 36.3 mmol). The resulting mixture was stirred at room temperature for 2 hours and quenched with saturated aqueous NaHCO₃ to pH-7. The mixture was extracted with EtOAc (300 mL×3). The combined organic layers were washed with saturated aqueous NaHCO₃ (50 mL) and brine (100 mL), dried over anhydrous sodium sulfate, filtered, and concentrated. The residue was purified by silica gel column chromatography, eluting with 5-60% EtOAc in petroleum ether, to afford the desired product, 5-(4-amino-2,6-difluoro-3-iodo-phenoxy)-2-methyl-benzonitrile (14.5 g, 97%) as a yellow solid. MS (ESI): 387.0 m/z (M+H)⁺.

### 5-[4-Amino-2,6-difluoro-3-(2-trimethylsilylethynyl)phenoxy]-2-methyl-benzonitrile

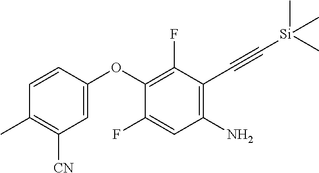

Step E: A mixture of 5-(4-amino-2,6-difluoro-3-iodo-phenoxy)-2-methyl-benzonitrile (14.5 g, 35.3 mmol), trimethylsilylacetylene (4.51 g, 45.9 mmol), Pd(PPh₃)₂Cl₂ (2.58 g, 3.53 mmol), CuI (0.672 g, 3.53 mmol) and triethylamine (6.48 mL, 45.9 mmol) in DMF (150 mL) was stirred under a nitrogen atmosphere at room temperature for 16 h. The mixture was diluted with EtOAc (500 mL) and filtered. The filtrate was washed with water (100 mL×3), brine (50 mL), dried over anhydrous sodium sulfate, filtered, and concentrated under reduced pressure. The residue was purified by silica gel chromatography, eluting with 0-10% EtOAc in petroleum ether, to afford the desired product, 5-[4-amino-2,6-difluoro-3-(2-trimethylsilylethynyl)phenoxy]-2-methyl-benzonitrile (12 g, 86%) as a yellow oil. MS (ESI): 357.1 m/z (M+H)⁺.

### 5-(4-Amino-3-ethynyl-2,6-difluoro-phenoxy)-2-methyl-benzonitrile

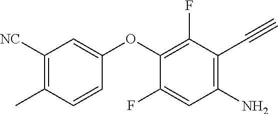 [SMILES: C#Cc1c(N)cc(F)c(Oc2ccc(C)c(C#N)c2)c1F]

Step F: A solution of 5-[4-amino-2,6-difluoro-3-(2-trimethylsilylethynyl)phenoxy]-2-methyl-benzonitrile (10 g, 25.2 mmol) and K₂CO₃ (6.98 g, 50.5 mmol) in MeOH (100 mL) was stirred at room temperature for 2 hours. The reaction mixture was diluted with EtOAc (500 mL) and washed with brine (100 mL), dried over anhydrous sodium sulfate, filtered, and concentrated under reduced pressure. The residue was purified by silica gel chromatography eluting with 0-40% EtOAc in petroleum ether to afford the desired product, 5-(4-amino-3-ethynyl-2,6-difluoro-phenoxy)-2-methyl-benzonitrile (7.02 g, 90%) as a yellow solid. MS (ESI): 285.0 m/z (M+H)⁺.

### 5-[(4,6-Difluoro-1H-indol-5-yl)oxy]-2-methyl-benzonitrile

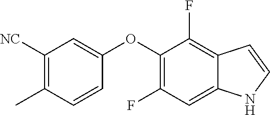 [SMILES: Cc1ccc(Oc2c(F)cc3[nH]ccc3c2F)cc1C#N]

Step G: A solution of 5-(4-amino-3-ethynyl-2,6-difluoro-phenoxy)-2-methyl-benzonitrile (7.02 g, 24.7 mmol) and potassium tert-butoxide (5.54 g, 49.4 mmol) in NMP (100 mL) was stirred at 80° C. for 2 hours. After cooling to room temperature, the reaction mixture was diluted with EtOAc (500 mL) and washed with brine (50 mL×2), dried over anhydrous sodium sulfate, filtered, and concentrated in vacuo. The residue was purified by silica gel chromatography, eluting with 0-40% acetone in petroleum ether, to afford the desired product, 5-[(4,6-difluoro-1H-indol-5-yl)oxy]-2-methyl-benzonitrile (4.0 g, 57%) as a yellow solid. MS (ESI): 285.0 m/z (M+H)⁺. ¹H NMR (400 MHz, CDCl₃): δ 8.41 (s, 1H), 7.24 (d, J=2.8 Hz, 1H), 7.22 (d, J=2.8 Hz, 1H), 7.15 (dd, J=8.8, 2.8 Hz, 1H), 7.10 (d, J=2.8 Hz, 1H), 7.07 (d, J=8.8 Hz, 1H), 6.65 (m, 1H), 2.49 (s, 3H) ppm.

### 5-[1-(Benzenesulfonyl)-4,6-difluoro-indol-5-yl]oxy-2-methyl-benzonitrile

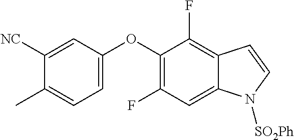

Step H: A solution of 5-[(4,6-difluoro-1H-indol-5-yl)oxy]-2-methyl-benzonitrile (2.0 g, 7.04 mmol), benzyl(triethyl)ammonium chloride (0.32 g, 1.41 mmol), NaOH (1.01 g, 25.3 mmol) and benzenesulfonyl chloride (2.98 g, 16.9 mmol) in dichloromethane (20 mL) was stirred at room temperature for 2 hours. The mixture was diluted with dichloromethane (100 mL), washed with brine (50 mL), dried over anhydrous sodium sulfate, filtered, and concentrated under reduced pressure. The residue was purified by silica gel column chromatography, eluting with 0-20% acetone in petroleum ether, to afford the desired product, 5-[1-(benzenesulfonyl)-4,6-difluoro-indol-5-yl]oxy-2-methyl-benzonitrile (2.8 g, 94%) as a white solid.

LC-Mass Method: Mobile Phase: A: water (10 mmol NH₄HCO3), B: CH₃CN; Gradient: 10% B increase to 95% B within 1.5 min; Flow Rate: 1.8 mL/min; Column: XBridge C18, 4.6*50 mm, 3.5 μm; Column Temperature: 50° C.; LC purity: 100% (214 nm), MS (ESI): no ionization observed; retention time: 2.34 min. ¹H NMR (400 MHz, DMSO-d₆): δ 8.16 (d, J=7.6 Hz, 2H), 8.00 (d, J=4.0 Hz, 1H), 7.88 (d, J=8.8 Hz, 1H), 7.78 (t, J=7.6 Hz, 1H), 7.67 (t, J=7.6 Hz, 2H), 7.50 (d, J=2.8 Hz, 1H), 7.41 (d, J=8.8 Hz, 1H), 7.26 (dd, J=8.8, 2.8 Hz, 1H), 7.00 (d, J=4.0 Hz, 1H), 2.42 (s, 3H) ppm.

### 5-[1-(Benzenesulfonyl)-4,6-difluoro-indol-5-yl]oxy-2-(bromomethyl)benzonitrile

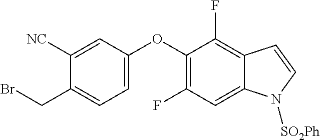

Step I: A mixture of 5-[1-(benzenesulfonyl)-4,6-difluoro-indol-5-yl]oxy-2-methyl-benzonitrile (1.0 g, 2.36 mmol), N-bromosuccinimide (629 mg, 3.53 mmol) and benzoyl benzene carboperoxoate (114 mg, 0.471 mmol) in 1,2-dichloroethane (20 mL) was heated at reflux for 16 hours under an argon atmosphere. The reaction mixture was cooled to room temperature and concentrated in vacuo to afford the crude desired product, 5-[1-(benzenesulfonyl)-4,6-difluoro-indol-5-yl]oxy-2-(bromomethyl)benzonitrile (0.85 g, 69%) as a white solid. MS (ESI): 503.0 & 505.0 m/z (M+H)⁺. ¹H NMR (400 MHz, CDCl₃) δ 7.94-7.92 (m, 2H), 7.73 (d, J=8.8 Hz, 1H), 7.63 (t, J=8.0 Hz, 1H), 7.61 (d, J=3.6 Hz, 1H), 7.54 (t, J=8.0 Hz, 2H), 7.48 (d, J=8.8 Hz, 1H), 7.17 (dd, J=8.8, 2.8 Hz, 1H), 7.11 (d, J=2.8 Hz, 1H), 6.77 (dd, J=3.6, 0.4 Hz, 1H), 4.60 (s, 2H) ppm.

### Methyl 2-(3-bromo-2-fluorophenyl)acetate

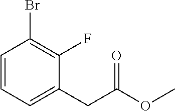

Step J: To a stirred solution of 2-(3-bromo-2-fluorophenyl)acetic acid (2.0 g, 8.58 mmol) in MeOH (20 mL) at room temperature was added dropwise concentrated sulfuric acid (0.5 mL) and the reaction was refluxed for 4 hours. The reaction mixture was concentrated under reduced pressure. The residue was dissolved in EtOAc (200 mL), washed with brine (50 mL×2), dried over anhydrous sodium sulfate, filtered, and concentrated to afford the desired product, methyl 2-(3-bromo-2-fluorophenyl)acetate (2.1 g, 99%) as a colorless oil. MS (ESI): 247.0 & 249.0 m/z (M+H)⁺.

### 2-(3-Bromo-2-fluoro-phenyl) acetohydrazide

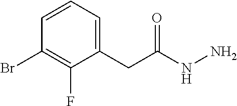 [SMILES: NNC(=O)Cc1cccc(Br)c1F]

Step K: A solution of methyl 2-(3-bromo-2-fluoro-phenyl)acetate (2.1 g, 8.50 mmol) and hydrazine hydrate (2.61 g, 51.0 mmol) in MeOH (20 mL) was heated at reflux for 3 hours. The reaction was concentrated in vacuo. The residue was diluted with EtOAc (200 mL), washed with brine (50 mL), dried over anhydrous sodium sulfate, filtered, and concentrated in vacuo to afford the desired product, 2-(3-bromo-2-fluoro-phenyl) acetohydrazide (1.8 g, 84%) as a white solid. MS (ESI): 247.1 & 249.1 m/z (M+H)⁺.

### N-[6-[1-(Benzenesulfonyl)-4,6-difluoro-indol-5-yl]oxy-1-imino-isoindolin-2-yl]-2-(3-bromo-2-fluoro-phenyl)acetamide

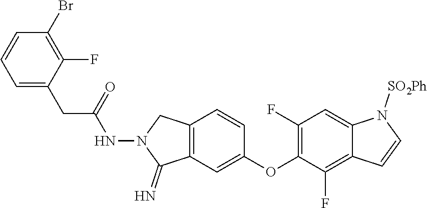

Step L: A mixture of 2-(3-bromo-2-fluoro-phenyl)acetohydrazide (product from Step K, 410 mg, 1.66 mmol) and 5-[1-(benzenesulfonyl)-4,6-difluoro-indol-5-yl]oxy-2-(bromomethyl) benzonitrile (product from Step I, 509 mg, 0.99 mmol) in MeOH (20 mL) was heated at reflux for 16 h. The mixture was concentrated in vacuo to afford the desired product, N-[6-[1-(benzenesulfonyl)-4,6-difluoro-indol-5-yl]oxy-1-imino-isoindolin-2-yl]-2-(3-bromo-2-fluoro-phenyl)acetamide (0.9 g, 51%) as a white solid. MS (ESI): 669.1 & 671.1 m/z (M+H)⁺

### 8-[1-(Benzenesulfonyl)-4,6-difluoro-indol-5-yl]oxy-2-[(3-bromo-2-fluoro-phenyl)methyl]-5H-[1,2,4]triazolo[5,1-a]isoindole

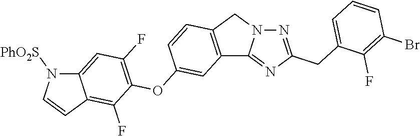

Step M: A mixture of N-[6-[I-(benzenesulfonyl)-4,6-difluoro-indol-5-yl]oxy-1-imino-isoindolin-2-yl]-2-(3-bromo-2-fluoro-phenyl)acetamide (0.9 g, 1.34 mmol) and POCl₃ (21.0 g, 134 mmol) was heated at reflux for 16 hours. The reaction mixture was concentrated in vacuo. Ice (50 g) was added to the residue and the mixture was extracted with EtOAc (100 mL). The organic layer was washed with brine (20 mL), dried over anhydrous sodium sulfate, filtered, and concentrated in vacuo. The residue was purified by column chromatography on silica gel, eluting with 10-60% acetone in petroleum ether, to afford the desired product, 8-[1-(benzenesulfonyl)-4,6-difluoro-indol-5-yl]oxy-2-[(3-bromo-2-fluoro-phenyl)methyl]-5H-[1,2,4]triazolo[5,1-a]isoindole (505 mg, 55%) as a yellow solid. MS (ESI): 651.0 & 653.0 m/z (M+H)⁺.

### Ethyl (E)-3-[3-[[8-[1-(benzenesulfonyl)-4,6-difluoro-indol-5-yl]oxy-5H-[1,2,4]triazolo[5,1-a]isoindol-2-yl]methyl]-2-fluoro-phenyl]prop-2-enoate

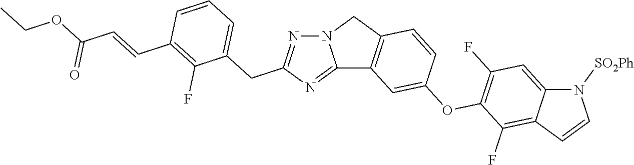

Step N: To a stirred solution of 8-[1-(benzenesulfonyl)-4,6-difluoro-indol-5-yl]oxy-2-[(3-bromo-2-fluoro-phenyl) methyl]-5H-[1,2,4]triazolo[5,1-a]isoindole (0.2 g, 0.307 mmol) and ethyl acrylate (92.2 mg, 0.921 mmol) in DMF (3 mL) at room temperature was added Pd(OAc)₂ (6.9 mg, 0.0307 mmol), Et₃N (155 mg, 1.54 mmol) and tris(o-tolyl)phosphine (18.7 mg, 0.0614 mmol), and the mixture was purged with argon for 1 min. The reaction tube was sealed and heated at 100° C. for 16 hours. After cooling to room temperature, the mixture was diluted with EtOAc (100 mL), washed with water (20 mL×3), dried over anhydrous sodium sulfate, filtered and concentrated in vacuo. The residue was purified by column chromatography on silica gel, eluting with 0-40% acetone in petroleum ether, to afford the desired product, ethyl (E)-3-[3-[[8-[1-(benzenesulfonyl)-4,6-difluoro-indol-5-yl]oxy-5H-[1,2,4]triazolo[5,1-a]isoindol-2-yl]methyl]-2-fluorophenyl]prop-2-enoate (101 mg, 40%) as a yellow solid. MS (ESI): 671.0 m/z (M+H)⁺.

### Ethyl 3-[3-[[8-[1-(benzenesulfonyl)-4,6-difluoro-indol-5-yl]oxy-5H-[1,2,4]triazolo[5,1-a]isoindol-2-yl]methyl]-2-fluoro-phenyl]propanoate

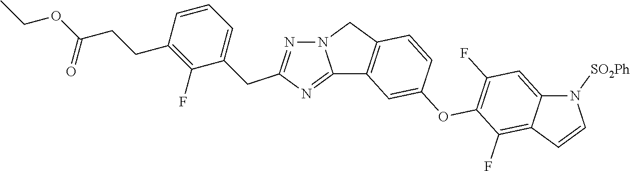

Step O: A suspension of ethyl (E)-3-[3-[[8-[1-(benzenesulfonyl)-4,6-difluoro-indol-5-yl]oxy-5H-[1,2,4]triazolo [5,1-a]isoindol-2-yl]methyl]-2-fluoro-phenyl]prop-2-enoate (90 mg, 0.134 mmol) and Raney nickel (39.4 mg, 0.671 mmol) in EtOH (100 mL) was stirred under a H₂ atmosphere at room temperature for 16 hours. The solids were filtered off and the filtrate was concentrated under reduced pressure to afford the crude desired product, ethyl 3-[3-[[8-[1-(benzenesulfonyl)-4,6-difluoro-indol-5-yl]oxy-5H-[1,2,4]triazolo[5,1-a]isoindol-2-yl]methyl]-2-fluorophenyl]propanoate (100 mg, 94%) as a yellow solid. MS (ESI): 673.2 m/z (M+H)⁺.

### 3-[3-[[8-[(4,6-Difluoro-1H-indol-5-yl)oxy]-5H-[1,2,4]triazolo[5,1-a]isoindol-2-yl]methyl]-2-fluoro-phenyl]propanoic acid

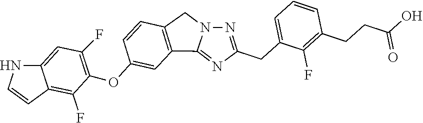 [SMILES: O=C(O)CCc1cccc(Cc2nc3n(n2)Cc2ccc(Oc4c(F)cc5[nH]ccc5c4F)cc2-3)c1F]

Step P: A mixture of ethyl 3-[3-[[8-[1-(benzenesulfonyl)-4,6-difluoro-indol-5-yl]oxy-5H-[1,2,4]triazolo [5,1-a]isoindol-2-yl]methyl]-2-fluoro-phenyl]propanoate (0.19 g, 0.28 mmol) and LiOH (135 mg, 5.64 mmol) in THE (20 mL) and water (6 mL) was stirred at 30° C. for 6 hours. After concentrating under reduced pressure, the mixture was acidified to pH-7 with 2N hydrochloric acid and extracted with EtOAc (50 mL×2). The combined organic extracts were dried over Na₂SO₄, filtered, and concentrated in vacuo. The residue was purified by prep-HPLC (NH₄HCO3/CH₃CN/water solvent system) and lyophilized to afford the desired product, 3-[3-[[8-[(4,6-difluoro-1H-indol-5-yl)oxy]-5H-[1,2,4]triazolo[5,1-a]isoindol-2-yl]methyl]-2-fluorophenyl]propanoic acid (65.4 mg, 46%) as a white solid. MS (ESI): 505.2 m/z (M+H)⁺. ¹H NMR (400 MHz, CD₃OD) δ 7.62 (d, J=8.4 Hz, 1H), 7.33 (d, J=3.2 Hz, 2H), 7.19-7.16 (m, 4H), 7.03 (t, J=7.6 Hz, 1H), 6.57 (d, J=2.8 Hz, 1H), 5.13 (s, 2H), 4.14 (s, 2H), 2.95 (t, J=7.6 Hz, 2H), 2.60 (t, J=7.6 Hz, 2H) ppm.

### Example 5. Synthesis of (S)-3-(3-(1-(9-((4,6-difluoro-1H-indol-5-yl)oxy)-5,6-dihydroimidazo[2,1-a]isoquinolin-3-yl)ethyl)-2-fluorophenyl)propanoic acid

### Example 6. Synthesis (R)-3-(3-(1-(9-((4,6-difluoro-1H-indol-5-yl)oxy)-5,6-dihydroimidazo[2,1-a]isoquinolin-3-yl)ethyl)-2-fluorophenyl)propanoic acid

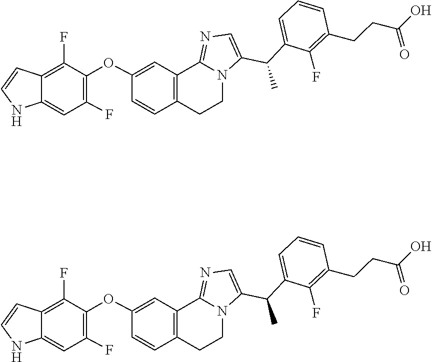 [SMILES: C[C@@H](c1cccc(CCC(=O)O)c1F)c1cnc2n1CCc1ccc(Oc3c(F)cc4[nH]ccc4c3F)cc1-2 ; C[C@H](c1cccc(CCC(=O)O)c1F)c1cnc2n1CCc1ccc(Oc3c(F)cc4[nH]ccc4c3F)cc1-2]

### 7-[4,6-Difluoro-1-(p-tolylsulfonyl)indol-5-yl]oxy-1-methoxy-3,4-dihydroisoquinoline

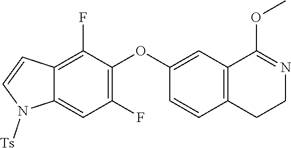 [SMILES: COC1=NCCc2ccc(Oc3c(F)cc4c(ccn4S(=O)(=O)c4ccc(C)cc4)c3F)cc21]

Step A: To a stirred solution of 7-[4,6-difluoro-1-(p-tolylsulfonyl)indol-5-yl]oxy-3,4-dihydro-2H-isoquinolin-1-one (Step G, Example 1; 1.5 g, 3.17 mmol) in dichloromethane (25 mL) was added sodium carbonate (1.68 g, 15.8 mmol) and Me₃OBF₄ (2.34 g, 15.8 mmol). The resulting mixture was stirred for 5 hours. Water (50 mL) was added, and the mixture was extracted with ethyl acetate (30 mL×3). The combined organic phase was washed with brine (20 mL), dried over sodium sulfate, filtered, and concentrated to give 7-[4,6-difluoro-1-(p-tolylsulfonyl)indol-5-yl]oxy-1-methoxy-3,4-dihydroisoquinoline (1.4 g, 82%). MS (ESI): 483.0 m/z (M+H)⁺.

### 7-((4,6-Difluoro-1-tosyl-1H-indol-5-yl)oxy)-3,4-dihydroisoquinolin-1-amine and 7-((4,6-difluoro-1H-indol-5-yl)oxy)-3,4-dihydroisoquinolin-1-amine

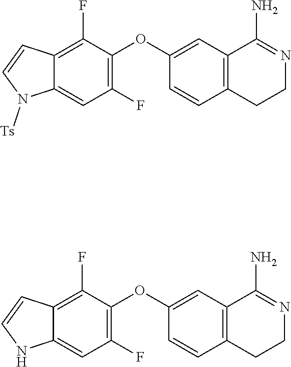 [SMILES: Cc1ccc(S(=O)(=O)n2ccc3c(F)c(Oc4ccc5c(c4)C(N)=NCC5)c(F)cc32)cc1 ; NC1=NCCc2ccc(Oc3c(F)cc4[nH]ccc4c3F)cc21]

Step B: A solution of 7-[4,6-difluoro-1-(p-tolylsulfonyl)indol-5-yl]oxy-1-methoxy-3,4-dihydroisoquinoline (1.4 g, 2.58 mmol) in 7N NH₃ solution in methanol (10 ml) was sealed in a reaction tube and heated at 70° C. for 15 hours. The solvent was removed under reduced pressure to afford the crude product mixture (1.3 g, 1:2 7-((4,6-difluoro-1-tosyl-1H-indol-5-yl)oxy)-3,4-dihydroisoquinolin-1-amine: 7-((4,6-difluoro-1H-indol-5-yl)oxy)-3,4-dihydroisoquinolin-1-amine). The crude product mixture was used without purification in the next reaction step. LCMS Method: Mobile Phase: A: water (0.1% TFA), B: acetonitrile (0.1% TFA); Gradient: 10% B for 0.2 min, increase to 90% B within 1.3 min, 90% B for 1.5 min; Flow Rate: 2 ml/min; Column: Sunshell C18 50*4.6 mm, 3.5 μm; Column Temperature: 50° C.; LC purity: 7-((4,6-difluoro-1-tosyl-1H-indol-5-yl)oxy)-3,4-dihydroisoquinolin-1-amine—32% (214 nm, retention time 1.72 minutes); MS (ESI): 468.1 ml (M+H)⁺; 7-((4,6-difluoro-1H-indol-5-yl)oxy)-3,4-dihydroisoquinolin-1-amine—46% (214 nm, retention time 1.48 minutes); MS (ESI): 314.1 m/z (M+H)⁺.

### (E)-2-(3-(3-Ethoxy-3-oxoprop-1-en-1-yl)-2-fluorophenyl)propanoic acid

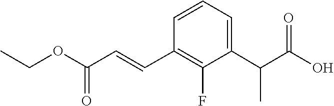 [SMILES: CCOC(=O)/C=C/c1cccc(C(C)C(=O)O)c1F]

Step C: A stirred solution of 2-(3-bromo-2-fluoro-phenyl)propanoic acid (3.1 g, 11.9 mmol), ethyl acrylate (5.97 g, 59.6 mmol), Pd(OAc)₂ (535 mg, 2.38 mmol), P(o-Tol)₃ (2.18 g, 7.15 mmol) and Et₃N (6.03 g, 59.6 mmol) in NMP (50 mL) was purged with nitrogen. The resulting mixture was stirred at 120° C. under nitrogen for 16 hours. The mixture was diluted with water (300 mL) and then acidified with 2M hydrochloric acid to pH-4 and extracted with ethyl acetate (100 mL×2). The combined organic phase was washed with water (50 ml) and brine (50 ml), dried over anhydrous sodium sulfate, filtered, and concentrated. The crude material was purified by flash column chromatography (eluting with 0 to 4% methanol in dichloromethane) to afford the title product (2.3 g, 63%). MS (ESI): 267.1 m/z (M+H)⁺.

### 2-(3-(3-ethoxy-3-oxopropyl)-2-fluorophenyl)propanoic acid

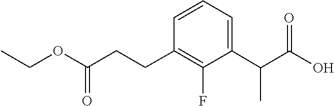 [SMILES: CCOC(=O)CCc1cccc(C(C)C(=O)O)c1F]

Step D: To a solution of (E)-2-(3-(3-ethoxy-3-oxoprop-1-en-1-yl)-2-fluorophenyl)propanoic acid (2.3 g, 7.43 mmol) in ethanol (30 mL) was added palladium on activated carbon (10%, 500 mg). The reaction mixture was stirred under hydrogen for 15 hours at 50° C. The catalyst was removed by filtration and washed with ethanol (20 mL). The filtrate was concentrated. The crude material was purified by flash column chromatography (eluting with 0 to 4% methanol in dichloromethane) to afford 2-(3-(3-ethoxy-3-oxopropyl)-2-fluorophenyl)propanoic acid as a light-yellow oil (1.8 g, 78%). MS (ESI): 269.2 m/z (M+H)⁺.

### Ethyl 3-[3-(3-bromo-1-methyl-2-oxo-propyl)-2-fluoro-phenyl]propanoate

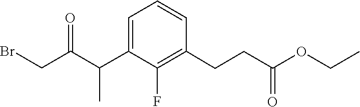 [SMILES: CCOC(=O)CCc1cccc(C(C)C(=O)CBr)c1F]

Step E: A solution of 2-[3-(3-ethoxy-3-oxo-propyl)-2-fluoro-phenyl]propanoic acid (1.8 g, 6.51 mmol) in SOCl₂ (5 mL) was stirred at 80° C. for 2 hours. The solvent was removed in vacuo. The residue was dissolved in acetonitrile (20 mL), then a solution of trimethylsilyldiazomethane in hexane (13 mL, 2M, 26 mmol) was added at 0° C. The mixture was stirred at room temperature overnight, then 36% hydrobromic acid (5.4 g) was added and stirring continued at room temperature for another hour. Water (100 mL) was added and then extracted with ethyl acetate (60 mL×2). The organic phase was washed with brine, dried over sodium sulfate, filtered, and concentrated. The residue was purified by silica gel column chromatography, eluting with 0-20% ethyl acetate in petroleum ether, to give ethyl 3-[3-(3-bromo-1-methyl-2-oxo-propyl)-2-fluoro-phenyl]propanoate as an oil (1.7 g, 67%). MS (ESI): 345.0 m/z (M+H)⁺.

### Ethyl 3-(3-(1-(9-((4,6-difluoro-1H-indol-5-yl)oxy)-5,6-dihydroimidazo[2,1-a]isoquinolin-3-yl)ethyl)-2-fluorophenyl)propanoate

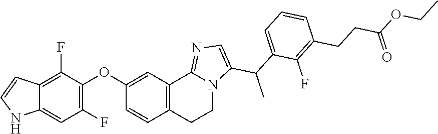 [SMILES: CCOC(=O)CCc1cccc(C(C)c2cnc3n2CCc2ccc(Oc4c(F)cc5[nH]ccc5c4F)cc2-3)c1F]

Step F: To a stirred solution of ethyl 3-(3-(4-bromo-3-oxobutan-2-yl)-2-fluorophenyl)propanoate (1.3 g, 2.45 mmol) in N,N-dimethylformamide (15 mL) was added sodium bicarbonate (411 mg, 4.89 mmol) and the product mixture from Step B (960 mg, 2.45 mmol). The mixture was stirred at 80° C. for 3 hours. The mixture was cooled to room temperature and diluted with water (60 mL), extracted with ethyl acetate (30 mL×3). The organic phase was washed with saturated aqueous lithium chloride (20 mL×2), brine, dried over sodium sulfate, filtered, and concentrated. The residue was added to a solution of tetrabutylammonium fluoride in tetrahydrofuran (10 mL, 10 mmol) and the mixture was stirred at 25° C. for 16 hours. The mixture was poured into water (50 mL), extracted with ethyl acetate (30 mL×3). The organic phase was washed with brine, dried over sodium sulfate, filtered, and concentrated. The residue was purified by silica gel column chromatography, eluting with 0-4% methanol in dichloromethane, to give ethyl 3-[3-[1-[9-[(4,6-difluoro-1H-indol-5-yl)oxy]-5,6-dihydroimidazo[2,1-a]isoquinolin-3-yl]ethyl]-2-fluoro-phenyl]propanoate as a light-yellow solid (610 mg, 50%). MS (ESI): 560.2 m/z (M+H)⁺.

### Ethyl (S)-3-(3-(1-(9-((4,6-difluoro-1H-indol-5-yl)oxy)-5,6-dihydroimidazo[2,1-a]isoquinolin-3-yl)ethyl)-2-fluorophenyl)propanoate and

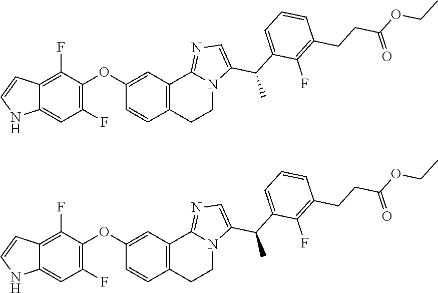 [SMILES: CCOC(=O)CCc1cccc([C@H](C)c2cnc3n2CCc2ccc(Oc4c(F)cc5[nH]ccc5c4F)cc2-3)c1F ; CCOC(=O)CCc1cccc([C@@H](C)c2cnc3n2CCc2ccc(Oc4c(F)cc5[nH]ccc5c4F)cc2-3)c1F]

Step G: The enantiomeric mixture, ethyl 3-[3-[1-[9-[(4,6-difluoro-1H-indol-5-yl)oxy]-5,6-dihydroimidazo[2,1-a]isoquinolin-3-yl]ethyl]-2-fluoro-phenyl]propanoate (220 mg, 0.651 mmol) was separated by chiral HPLC into its constituent enantiomers. The absolute configurations of both enantiomers were assigned arbitrarily. Thus, the absolute configuration of the faster eluting enantiomer was assigned as ethyl 3-[3-[(1S)-1-[9-[(4,6-difluoro-1H-indol-5-yl)oxy]-5,6-dihydroimidazo[2,1-a]isoquinolin-3-yl]ethyl]-2-fluoro-phenyl]propanoate (75 mg, a white solid), MS (ESI): 560.2 m/z (M+H)⁺, and that of the slower eluting enantiomer as ethyl 3-[3-[(1R)-1-[9-[(4,6-difluoro-1H-indol-5-yl)oxy]-5,6-dihydroimidazo [2,1-a]isoquinolin-3-yl]ethyl]-2-fluoro-phenyl]propanoate (72 mg, a white solid). MS (ESI): 560.2 m/z (M+H)⁺ observed for both enantiomers.

### Chiral HPLC Separation Conditions:

Instrument: SFC-200 (Thar, Waters)
Column: (S,S)-Whelk-O1 20*250 mm, 10 μm (Regis)
Column temperature: 35° C.
Mobile phase: 1:1 CO₂/methanol (0.2% ammonia in methanol)
Flow rate: 120 g/minute; Back pressure: 100 bar
Detection wavelength: 214 nm; Cycle time: 8.6 minutes
Sample solution: 200 mg dissolved in 15 ml methanol; Injection volume: 5 ml

### (S)-3-(3-(1-(9-((4,6-Difluoro-1H-indol-5-yl)oxy)-5,6-dihydroimidazo[2,1-a]isoquinolin-3-yl)ethyl)-2 fluorophenyl)propanoic acid

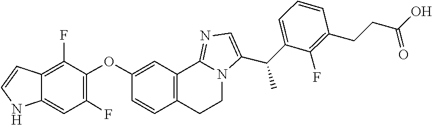 [SMILES: C[C@@H](c1cccc(CCC(=O)O)c1F)c1cnc2n1CCc1ccc(Oc3c(F)cc4[nH]ccc4c3F)cc1-2]

Step H: To a stirred solution of ethyl 3-[3-[(1S)-1-[9-[(4,6-difluoro-1H-indol-5-yl)oxy]-5,6-dihydroimidazo[2,1-a]isoquinolin-3-yl]ethyl]-2-fluoro-phenyl]propanoate (72 mg, 0.125 mmol) in a mixture of methanol (1.5 mL) and tetrahydrofuran (0.5 mL) was added lithium hydroxide monohydrate (15.7 mg, 0.374 mmol) in water (0.5 mL). The resulting mixture was stirred at 30° C. for 6 hours. After the reaction was completed, the solvent was removed and then water (10 mL) was added, and the pH was adjusted to 5-6 with 1N hydrochloric acid. The formed precipitate was collected by filtration and then dried to afford (S)-3-(3-(1-(9-((4,6-difluoro-1H-indol-5-yl)oxy)-5,6-dihydroimidazo[2,1-a]isoquinolin-3-yl)ethyl)-2-fluorophenyl)propanoic acid (Example 5) as a white solid (62 mg, 91%). MS (ESI): 532.2 m/z (M+H)⁺. ¹H NMR (400 MHz, CD₃OD): δ 7.42 (d, J=2.8 Hz, 1H), 7.29-7.26 (m, 2H), 7.13-7.09 (m, 2H), 7.05-6.94 (m, 3H), 6.91 (s, 1H), 6.54 (d, J=2.8 Hz, 1H), 4.41 (q, J=7.2 Hz, 1H), 4.19 (t, J=6.4 Hz, 2H), 3.14 (t, J=6.8 Hz, 2H), 2.95 (t, J=7.6 Hz, 2H), 2.62 (t, J=7.6 Hz, 2H), 1.57 (d, J=7.2 Hz, 3H) ppm.

### (R)-3-(3-(1-(9-((4,6-Difluoro-1H-indol-5-yl)oxy)-5,6-dihydroimidazo[2,1-a]isoquinolin-3-yl)ethyl)-2-fluorophenyl)propanoic acid

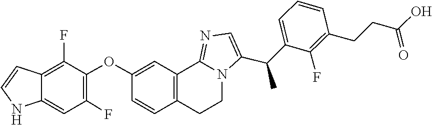 [SMILES: C[C@H](c1cccc(CCC(=O)O)c1F)c1cnc2n1CCc1ccc(Oc3c(F)cc4[nH]ccc4c3F)cc1-2]

Step I: To a stirred solution of ethyl 3-[3-[(1R)-1-[9-[(4,6-difluoro-1H-indol-5-yl)oxy]-5,6-dihydroimidazo[2,1-a]isoquinolin-3-yl]ethyl]-2-fluoro-phenyl]propanoate (72 mg, 0.125 mmol) in a mixture of methanol (1.5 mL) and tetrahydrofuran (0.5 mL) was added a solution of lithium hydroxide monohydrate (15.7 mg, 0.374 mmol) in water (0.5 mL). The resulting mixture was stirred at 30° C. for 6 hours. After the reaction was completed, the solvent was removed, water (10 mL) was added, and the pH was adjusted to 5-6 with 1N hydrochloric acid. The precipitate was collected by filtration and then dried to afford (R)-3-(3-(1-(9-((4,6-difluoro-1H-indol-5-yl)oxy)-5,6-dihydroimidazo[2,1-a]isoquinolin-3-yl)ethyl)-2-fluorophenyl)propanoic acid (Example 6) (61 mg, 90%). MS (ESI): 532.2 m/z (M+H)⁺. ¹H NMR (400 MHz, CD₃OD): δ 7.41 (d, J=2.8 Hz, 1H), 7.38 (d, J=8.4 Hz, 1H), 7.31 (d, J=3.2 Hz, 1H), 7.19 (s, 1H), 7.18-7.07 (m, 3H), 7.05-7.01 (m, 2H), 6.54 (d, J=3.2 Hz, 1H), 4.46 (q, J=6.8 Hz, 1H), 4.30-4.24 (m, 2H), 3.22 (t, J=7.2 Hz, 2H), 2.95 (t, J=7.6 Hz, 2H), 2.60 (t, J=7.6 Hz, 2H), 1.60 (d, J=7.2 Hz, 3H) ppm.

### Example 7. Synthesis of 3-(3-(1-(9-((4,6-difluoro-1H-indol-5-yl)oxy)imidazo[2,1-a]isoquinolin-3-yl)ethyl)-2-fluorophenyl)propanoic acid

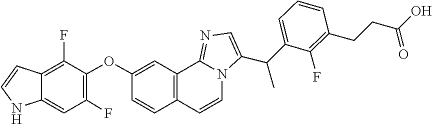 [SMILES: CC(c1cccc(CCC(=O)O)c1F)c1cnc2c3cc(Oc4c(F)cc5[nH]ccc5c4F)ccc3ccn12]

### Ethyl 3-(3-(1-(9-((4,6-difluoro-1H-indol-5-yl)oxy)imidazo[2,1-a]isoquinolin-3-yl)ethyl)-2-fluorophenyl)propanoate

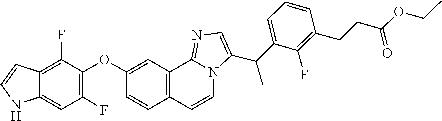 [SMILES: CCOC(=O)CCc1cccc(C(C)c2cnc3c4cc(Oc5c(F)cc6[nH]ccc6c5F)ccc4ccn23)c1F]

Step A: Palladium on activated carbon (10 wt. %, 880 mg) was added, under a nitrogen atmosphere, to a solution of ethyl 3-[3-[1-[9-[(4,6-difluoro-1H-indol-5-yl)oxy]-5,6-dihydroimidazo[2,1-a]isoquinolin-3-yl]ethyl]-2-fluorophenyl]propanoate (Step F, Example 5, 490 mg, 0.867 mmol) in Decalin (25 ml). The mixture was stirred at 200° C. for 6 hours. The solvent was concentrated and the residue was purified by silica gel column chromatography, eluting with 0-4% methanol in dichloromethane, to give the desired product, ethyl 3-[3-[1-[9-[(4,6-difluoro-1H-indol-5-yl)oxy]imidazo[2,1-a]isoquinolin-3-yl]ethyl]-2-fluoro-phenyl]propanoate (190 mg, 39%) as a solid. MS (ESI): 558.0 m/z (M+H)⁺.

### 3-(3-(1-(9-((4,6-Difluoro-1H-indol-5-yl)oxy)imidazo[2,1-a]isoquinolin-3-yl)ethyl)-2-fluorophenyl)propanoic acid

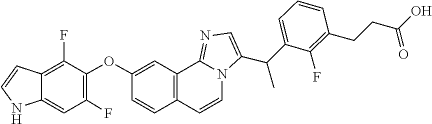 [SMILES: CC(c1cccc(CCC(=O)O)c1F)c1cnc2c3cc(Oc4c(F)cc5[nH]ccc5c4F)ccc3ccn12]

Step B: To a stirred solution of ethyl 3-[3-[1-[9-[(4,6-difluoro-1H-indol-5-yl)oxy]imidazo[2,1-a]isoquinolin-3-yl]ethyl]-2-fluoro-phenyl]propanoate (175 mg, 0.31 mmol) in methanol (3 mL) and tetrahydrofuran (1 mL) was added a solution of lithium hydroxide monohydrate (40 mg, 0.94 mmol) in water (1 mL) and then the mixture was stirred at room temperature for 15 hours. The solvent was removed under reduced pressure and the residue was dissolved in water. The aqueous phase was neutralized with 1M hydrochloric acid and then extracted with ethyl acetate (10 mL×3). The organic phase was concentrated under reduced pressure and the residue was purified by prep-HPLC to give 3-(3-(1-(9-((4,6-difluoro-1H-indol-5-yl)oxy)imidazo[2,1-a]isoquinolin-3-yl)ethyl)-2-fluorophenyl)propanoic acid (45 mg, 27%) as a light white solid. MS (ESI): 530.1 m/z (M+H)⁺. ¹H NMR (400 MHz, DMSO-d₆): δ 11.69 (s, 1H), 8.21 (d, J=7.2 Hz, 1H), 7.89 (d, J=8.8 Hz, 1H), 7.63 (s, 1H), 7.55 (d, J=2.4 Hz, 1H), 7.51 (t, J=2.8 Hz, 1H), 7.41-7.35 (m, 2H), 7.20 (d, J=7.2 Hz, 1H), 7.15-7.11 (m, 1H), 7.09-7.06 (m, 1H), 7.03 (d, J=7.2 Hz, 1H), 6.59 (t, J=2.0 Hz, 1H), 4.49 (q, J=6.8 Hz, 1H), 2.84 (t, J=7.6 Hz, 2H), 2.52 (t, J=7.6 Hz, 2H), 1.57 (d, J=7.2 Hz, 3H) ppm.

### Example 8. Synthesis of 3-(3-(1-(10-((4,6-difluoro-1H-indol-5-yl)oxy)-6,7-dihydro-5H-benzo[c]imidazo[1,2-a]azepin-2-yl)ethyl)-2-fluorophenyl)propanoic acid

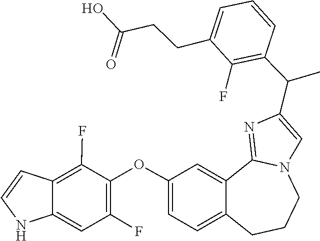 [SMILES: CC(c1cn2c(n1)-c1cc(Oc3c(F)cc4[nH]ccc4c3F)ccc1CCC2)c1cccc(CCC(=O)O)c1F]

### 5-(2,6-Difluoro-4-nitrophenoxy)-2-fluorobenzonitrile

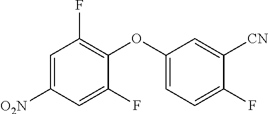 [SMILES: N#Cc1cc(Oc2c(F)cc([N+](=O)[O-])cc2F)ccc1F]

Step A: To a stirred solution of 1,2,3-trifluoro-5-nitrobenzene (45 g, 0.254 mol) and 2-fluoro-5-hydroxybenzonitrile (38.3 g, 0.28 mol) in DMF (200 mL) was added K₂CO₃ (70 g, 0.5 mol). The resulting mixture was heated for 2 hours at 100° C. To the cooled reaction was added water (1500 mL), and the mixture extracted with EtOAc (1000 mL×2). The combined organic phase was washed with brine (500 mL×3), dried over anhydrous Na₂SO₄, filtered and concentrated under reduced pressure to afford the desired product, 5-(2,6-difluoro-4-nitrophenoxy)-2-fluorobenzonitrile (74 g, 99%) as a yellow solid. ¹H NMR (500 MHz, DMSO-d₆): δ 8.03-7.98 (m, 2H), 7.28-7.23 (m, 2H), 7.21-7.19 (m, 1H) ppm.

### 5-(4-Amino-2,6-difluorophenoxy)-2-fluorobenzonitrile

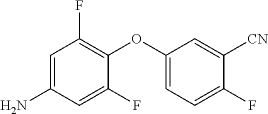 [SMILES: N#Cc1cc(Oc2c(F)cc(N)cc2F)ccc1F]

Step B: To a suspension of 5-(2,6-difluoro-4-nitrophenoxy)-2-fluorobenzonitrile (37 g, 126 mmol) in EtOH (450 mL) was added iron power (28.1 g, 543 mmol) and a solution of NH₄C1 (53.8 g, 1.01 mol) in water (150 mL). The resulting mixture was heated to reflux for 4 hours. The reaction mixture was filtered through Celite, and the filtrate was concentrated under reduced pressure to give a residue, which was dissolved in ethyl acetate (1 L). The organic phase was washed with water (200 mL×3), brine (200 mL), dried over anhydrous Na₂SO₄, filtered, and concentrated in vacuo to afford the desired product, 5-(4-amino-2,6-difluorophenoxy)-2-fluorobenzonitrile (33.2 g, quantitative yield) as a yellow solid. ¹H NMR (500 MHz, CDCl₃) δ 7.23-7.20 (m, 1H), 7.14 (t, J=8.5 Hz, 1H), 7.08 (dd, J=5.0, 3.5 Hz, 1H), 6.33-6.28 (m, 2H), 3.90 (br, s 2H) ppm.

### 5-(4-Amino-2,6-difluoro-3-iodophenoxy)-2-fluorobenzonitrile

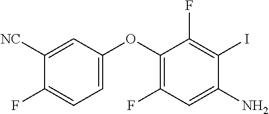 [SMILES: N#Cc1cc(Oc2c(F)cc(N)c(I)c2F)ccc1F]

Step C: A solution of 5-(4-amino-2,6-difluorophenoxy)-2-fluorobenzonitrile (33.2 g, 126 mmol) and N-iodosuccinimide (31.1 g, 126 mmol) in AcOH (250 mL) was stirred at room temperature for 1 hour. The solution was concentrated under reduced pressure. The residue was suspended in saturated aqueous NaHCO₃ (500 mL), and the resulting mixture was extracted with ethyl acetate (500 mL×3). The organic phase was washed with brine (200 mL×3), dried over anhydrous Na₂SO₄, filtered and concentrated. The residue was purified by silica gel column chromatography (eluting with 4/1 petroleum ether/ethyl acetate) to afford the desired product, 5-(4-amino-2,6-difluoro-3-iodophenoxy)-2-fluorobenzonitrile (45.5 g, 92%) as a yellow solid.

¹H NMR (500 MHz, DMSO-d₆): δ 7.63 (dd, J=5.0, 3.5 Hz, 1H), 7.49 (t, J=9.0 Hz, 1H), 7.39-7.36 (m, 1H), 6.63 (dd, J=13, 2.0 Hz, 1H), 5.88 (br s, 2H) ppm.

### 5-[4-Amino-2,6-difluoro-3-(2-trimethylsilylethynyl)phenoxy]-2-fluorobenzonitrile

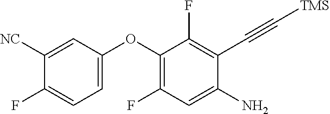 [SMILES: C[Si](C)(C)C#Cc1c(N)cc(F)c(Oc2ccc(F)c(C#N)c2)c1F]

Step D: To a solution of 5-(4-amino-2,6-difluoro-3-iodophenoxy)-2-fluorobenzonitrile (61 g, 156 mmol) in DMF (300 mL) was added Pd(dppf)Cl₂ (3.43 g, 4.7 mmol), CuI (2.98 g, 15.6 mmol) and Et₃N (23.7 g, 235 mmol), followed by the addition of ethynyl(trimethyl)silane (28 mL, 187 mmol). The resulting mixture was stirred at ambient temperature overnight under a nitrogen atmosphere. The reaction was quenched with water (500 mL) and extracted with ethyl acetate (500 mL×3). The combined organic phase was washed with brine (150 mL×3), dried over anhydrous Na₂SO₄, filtered, and concentrated in vacuo. The residue was purified by silica gel column chromatography (eluting with 1/3/3 petroleum ether/EtOAc/DCM) to afford the desired product, 5-[4-amino-2,6-difluoro-3-(2-trimethylsilylethynyl)phenoxy]-2-fluorobenzonitrile (42 g, 75%) as a yellow solid. MS (ESI): 361 m/z (M+H)⁺.

### 5-[(4,6-Difluoro-1H-indol-5-yl)oxy]-2-fluorobenzonitrile

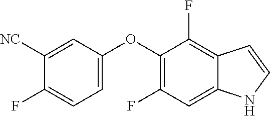 [SMILES: N#Cc1cc(Oc2c(F)cc3[nH]ccc3c2F)ccc1F]

Step E: A mixture of 5-[4-amino-2,6-difluoro-3-(2-trimethylsilylethynyl)phenoxy]-2-fluoro-benzonitrile (28.0 g, 77.7 mmol) and CuI (29.6 g, 155 mmol) in DMF (250 mL) was flushed with argon for 2 minutes and heated for 5 hours at 100° C. under argon. The insoluble materials were removed by suction filtration and the filtrate was diluted with ethyl acetate (1500 mL). The organic phase was washed with brine (300 mL×5), dried over anhydrous Na₂SO₄, filtered, and concentrated. The residue was purified by silica gel column chromatography (eluting with 4/1 petroleum ether/ethyl acetate) to afford the desired product, 5-[(4,6-difluoro-1H-indol-5-yl)oxy]-2-fluorobenzonitrile (18 g, 80%) as a yellow solid. ¹H NMR (500 MHz, DMSO-d₆) δ 11.64 (br s, 1H), 7.62 (dd, J=5.0, 3.5 Hz, 1H), 7.51-7.47 (m, 2H), 7.39-7.36 (m, 1H), 7.32 (d, J=10.0 Hz, 1H), 6.58 (t, J=2.0 Hz, 1H) ppm.

### 5-((4,6-Difluoro-1-((2-(trimethylsilyl)ethoxy)methyl)-1H-indol-5-yl)oxy)-2-fluorobenzonitrile

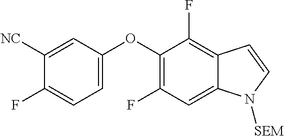 [SMILES: C[Si](C)(C)CCOCn1ccc2c(F)c(Oc3ccc(F)c(C#N)c3)c(F)cc21]

Step F: To a stirred solution of 5-[(4,6-difluoro-1H-indol-5-yl)oxy]-2-fluoro-benzonitrile (1.00 eq, 10.00 g, 34.7 mmol) in DMF (30 mL) was added sodium hydride (1.20 eq, 999 mg, 41.6 mmol). The mixture was stirred at 0° C. for 30 minutes before 2-(trimethylsilyl)ethoxymethyl chloride (1.10 eq, 6.8 mL, 38.2 mmol) was added. The mixture was further stirred at room temperature for 2 hours, diluted with water (100 mL), and extracted with EtOAc (80 mL×2). The organic layer was washed with water (100 mL×2) and brine (80 mL), dried over Na₂SO₄ and concentrated to afford the desired product, 5-[4,6-difluoro-1-(2-trimethylsilylethoxymethyl)indol-5-yl]oxy-2-fluoro-benzonitrile (14.00 g, 33.5 mmol, 96%). MS (ESI): 419 m/z (M+H)⁺.

### 5-((4,6-Difluoro-1-((2-(trimethylsilyl)ethoxy)methyl)-1H-indol-5-yl)oxy)-2-hydroxybenzonitrile

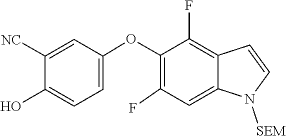 [SMILES: C[Si](C)(C)CCOCn1ccc2c(F)c(Oc3ccc(O)c(C#N)c3)c(F)cc21]

Step G: To a stirred solution of 5-[4,6-difluoro-1-(2-trimethylsilylethoxymethyl)indol-5-yl]oxy-2-fluoro-benzonitrile (1.00 eq, 7.00 g, 16.7 mmol) in DMF (50 mL) was added 2-(methylsulfonyl)ethanol (1.20 eq, 2492 mg, 20.1 mmol). The mixture was cooled to 0° C. before sodium hydride (1.30 eq, 522 mg, 21.7 mmol) was added. The mixture was warmed to room temperature and stirred for 2 hours. The reaction was diluted with water (100 mL) and extracted with EtOAc (80 mL×2). The organic layer was washed with saturated aqueous LiCl (80 mL×2) and brine (90 mL), dried over Na₂SO₄, and concentrated. The residue was purified by column chromatography on silica, eluting with 0-30% EtOAc in petroleum ether, to afford the desired product, 5-[4,6-difluoro-1-(2-trimethylsilylethoxymethyl)indol-5-yl]oxy-2-hydroxy-benzonitrile (4.50 g, 10.8 mmol, 65%). MS (ESI): 417 m/z (M+H)⁺.

### 2-Cyano-4-((4,6-difluoro-1-((2-(trimethylsilyl)ethoxy)methyl)-1H-indol-5-yl)oxy)phenyl trifluoromethanesulfonate

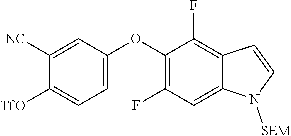 [SMILES: C[Si](C)(C)CCOCn1ccc2c(F)c(Oc3ccc(OS(=O)(=O)C(F)(F)F)c(C#N)c3)c(F)cc21]

Step H: To a solution of 5-[4,6-difluoro-1-(2-trimethylsilylethoxymethyl)indol-5-yl]oxy-2-hydroxy-benzonitrile (1.00 eq, 4.60 g, 11.0 mmol) in DCM (50 mL) was added N,N-diethylethanamine (1.50 eq, 2.3 mL, 16.6 mmol). The mixture was cooled to 0° C. and trifluoromethanesulfonic anhydride (1.20 eq, 2.2 mL, 13.3 mmol) was added dropwise. The reaction was stirred at 0° C. for 10 min and quenched with saturated aqueous NaHCO₃ (50 mL). The organic layer was washed with brine (50 mL), dried over Na₂SO₄, and concentrated. The residue was purified by silica gel column chromatography, eluting with 0-15% EtOAc in petroleum ether, to afford the desired product, 2-cyano-4-((4,6-difluoro-1-((2-(trimethylsilyl)ethoxy)methyl)-1H-indol-5-yl)oxy)phenyl trifluoromethanesulfonate (3.90 g, 7.11 mmol, 64%) as a yellow oil. MS (ESI): 549 m/z (M+H)⁺.

### 5-((4,6-Difluoro-1-((2-(trimethylsilyl)ethoxy)methyl)-1H-indol-5-yl)oxy)-2-vinylbenzonitrile

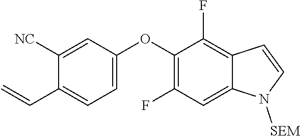 [SMILES: C=Cc1ccc(Oc2c(F)cc3c(ccn3COCC[Si](C)(C)C)c2F)cc1C#N]

Step I: To a stirred solution of [2-cyano-4-[4,6-difluoro-1-(2-trimethylsilylethoxymethyl)indol-5-yl]oxy-phenyl]trifluoromethanesulfonate (1.00 eq, 3.90 g, 7.11 mmol) in 1,4-Dioxane (20 mL) and water (2 mL) was added potassium vinyl trifluoroborate (1.10 eq, 1048 mg, 7.82 mmol), potassium carbonate (3.00 eq, 2991 mg, 21.3 mmol), and Pd(dppf)Cl₂ (0.10 eq, 576 mg, 0.711 mmol). The mixture was stirred at 90° C. for 16 hours, and concentrated. The residue was purified by silica column chromatography, eluting with 0-30% EtOAc in petroleum ether, to afford the desired product, 5-((4,6-difluoro-1-((2-(trimethylsilyl)ethoxy)methyl)-1H-indol-5-yl)oxy)-2-vinylbenzonitrile (2.00 g, 4.69 mmol, 66%) as a brown oil. MS (ESI): 427 m/z (M+H)⁺.

### 5-((4,6-Difluoro-1-((2-(trimethylsilyl)ethoxy)methyl)-1H-indol-5-yl)oxy)-2-vinylbenzothioamide

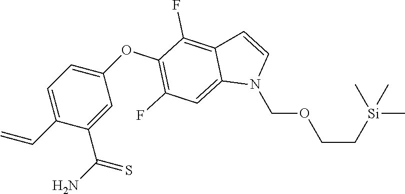 [SMILES: C=Cc1ccc(Oc2c(F)cc3c(ccn3COCC[Si](C)(C)C)c2F)cc1C(N)=S]

Step J: To a stirred solution of 5-[4,6-difluoro-1-(2-trimethylsilylethoxymethyl)indol-5-yl]oxy-2-vinyl-benzonitrile (1.00 eq., 2.00 g, 4.69 mmol) in DMF (20 mL) was added magnesium chloride (4.00 eq, 1786 mg, 18.8 mmol) and sodium hydrosulfide (8.00 eq, 2179 mg, 37.5 mmol). The mixture was stirred at room temperature for 16 hours, quenched with water (50 mL), and extracted with EtOAc (50 mL×2). The organic layer was washed with saturated aqueous LiCl (80 mL×2) and brine (50 mL), dried over Na₂SO₄ and concentrated. The crude was purified by silica gel column chromatography, eluting with 0-30% EtOAc in petroleum ether, to afford the desired product, 5-[4,6-difluoro-1-(2-trimethylsilylethoxymethyl)indol-5-yl]oxy-2-vinyl-benzenecarbothioamide (450 mg, 0.98 mmol, 21%) as a brown solid. MS (ESI): 461 m/z (M+H)⁺.

### Methyl 5-((4,6-difluoro-1-((2-(trimethylsilyl)ethoxy)methyl)-1H-indol-5-yl)oxy)-2-vinylbenzimidothioate hydroiodide

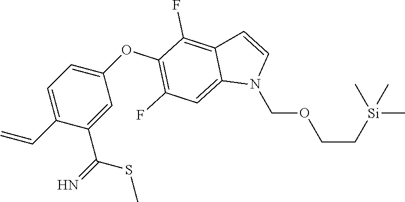 [SMILES: C=Cc1ccc(Oc2c(F)cc3c(ccn3COCC[Si](C)(C)C)c2F)cc1C(=N)SC]

Step K: To a stirred solution of 5-[4,6-difluoro-1-(2-trimethylsilylethoxymethyl)indol-5-yl]oxy-2-vinyl-benzenecarbothioamide (1.00 eq, 450 mg, 0.977 mmol) in acetone (10 mL) was added methyl iodide (5.00 eq, 0.31 mL, 4.88 mmol). The mixture was stirred at 50° C. for 16 hours and concentrated to afford the desired product, hydroiodide salt of methyl 5-[4,6-difluoro-1-(2-trimethylsilylethoxymethyl)indol-5-yl]oxy-2-vinyl-benzenecarboximidothioate (460 mg, 0.969 mmol, 99%) as a brown oil. MS (ESI): 475 m/z (M+H)⁺.

### N-Allyl-5-[4,6-difluoro-1-(2-trimethylsilylethoxymethyl)indol-5-yl]oxy-2-vinyl-benzamidine

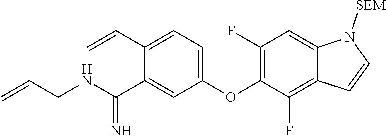 [SMILES: C=CCNC(=N)c1cc(Oc2c(F)cc3c(ccn3COCC[Si](C)(C)C)c2F)ccc1C=C]

Step L: To a stirred solution of the hydroiodide salt of methyl 5-[4,6-difluoro-1-(2-trimethylsilylethoxymethyl)indol-5-yl]oxy-2-vinyl-benzenecarboximidothioate (1.00 eq, 588 mg, 0.976 mmol) in pyridine (10 mL) was added 2-propen-1-amine hydrochloride (1.10 eq, 100 mg, 1.07 mmol). The mixture was stirred at 90° C. for 16 hours and concentrated. The residue was purified by silica gel column chromatography, eluting with 0-100% methanol in DCM, to afford the desired product, N-allyl-5-[4,6-difluoro-1-(2-trimethylsilylethoxymethyl)indol-5-yl]oxy-2-vinyl-benzamidine (300 mg, 0.62 mmol, 64%). MS (ESI): 484 m/z (M+H)⁺.

### Ethyl 3-[3-[1-[1-allyl-2-[5-[4,6-difluoro-1-(2-trimethylsilylethoxymethyl)indol-5-yl]oxy-2-vinyl-phenyl]imidazol-4-yl]ethyl]-2-fluoro-phenyl]propanoate

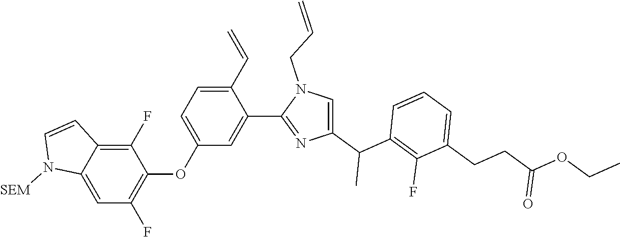 [SMILES: C=CCn1cc(C(C)c2cccc(CCC(=O)OCC)c2F)nc1-c1cc(Oc2c(F)cc3c(ccn3COCC[Si](C)(C)C)c2F)ccc1C=C]

Step M: To a stirred solution of ethyl 3-[3-(3-bromo-1-methyl-2-oxo-propyl)-2-fluoro-phenyl]propanoate (Step E, Example 5) (1.00 eq, 214 mg, 0.620 mmol) in THE (10 mL) was added N-allyl-5-[4,6-difluoro-1-(2-trimethylsilylethoxymethyl)indol-5-yl]oxy-2-vinyl-benzamidine (1.00 eq, 300 mg, 0.620 mmol) and sodium bicarbonate (3.00 eq, 156 mg, 1.86 mmol). The mixture was stirred at 70° C. for 16 hours, quenched with water (30 mL), and extracted with EtOAc (30 mL×2). The organic layer was washed with water (50 mL×2), brine (50 mL), dried over Na₂SO₄, and concentrated. The residue was purified by silica column chromatography, eluting with 0-100% MeOH in DCM, to afford the desired product, ethyl 3-[3-[1-[1-allyl-2-[5-[4,6-difluoro-1-(2-trimethylsilylethoxymethyl)indol-5-yl]oxy-2-vinyl-phenyl]imidazol-4-yl]ethyl]-2-fluoro-phenyl]propanoate (100 mg, 0.137 mmol, 22%) as a brown solid. MS (ESI): 730 m/z (M+H)⁺.

### Ethyl 3-[3-[1-[10-[4,6-difluoro-1-(2-trimethylsilylethoxynethyl)indol-5-yl]oxy-5H-imidazo[2,1-a][2]benzazepin-2-yl]ethyl]-2-fluoro-phenyl]propanoate

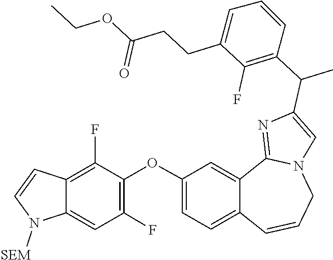 [SMILES: CCOC(=O)CCc1cccc(C(C)c2cn3c(n2)-c2cc(Oc4c(F)cc5c(ccn5COCC[Si](C)(C)C)c4F)ccc2C=CC3)c1F]

Step N: To a stirred solution of ethyl 3-[3-[1-[1-allyl-2-[5-[4,6-difluoro-1-(2-trimethylsilylethoxymethyl)indol-5-yl]oxy-2-vinyl-phenyl]imidazol-4-yl]ethyl]-2-fluoro-phenyl]propanoate (1.00 eq, 100 mg, 0.137 mmol) in 1,2-dichloroethane (200 mL) was added 2nd generation Grubbs catalyst (0.200 eq, 23 mg, 0.0274 mmol). The mixture was stirred at 90° C. for 16 hours, and concentrated. The residue was purified by silica gel column chromatography, eluting with 0-30% EtOAc in petroleum ether, to afford the desired product, ethyl 3-[3-[1-[10-[4,6-difluoro-1-(2-trimethylsilylethoxymethyl)indol-5-yl]oxy-5H-imidazo[2,1-a][2]benzazepin-2-yl]ethyl]-2-fluoro-phenyl]propanoate (80 mg, 0.11 mmol, 83%) as a solid. MS (ESI): 702 m/z (M+H)⁺.

### Ethyl 3-[3-[1-[10-[4,6-difluoro-1-(2-trimethylsilylethoxymethyl)indol-5-yl]oxy-6,7-dihydro-5H-imidazo[2,1-a][2]benzazepin-2-yl]ethyl]-2-fluoro-phenyl]propanoate

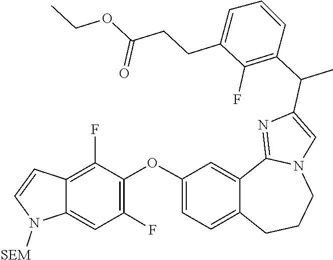 [SMILES: CCOC(=O)CCc1cccc(C(C)c2cn3c(n2)-c2cc(Oc4c(F)cc5c(ccn5COCC[Si](C)(C)C)c4F)ccc2CCC3)c1F]

Step O: To a stirred solution of ethyl 3-[3-[1l-[10-[4,6-difluoro-1-(2-trimethylsilylethoxymethyl)indol-5-yl]oxy-5H-imidazo[2,1-a][2]benzazepin-2-yl]ethyl]-2-fluoro-phenyl]propanoate (1.00 eq, 80 mg, 0.11 mmol) in ethanol (5 mL) was added Pd on activated carbon (10 wt. %, 20 mg). The mixture was stirred under a hydrogen atmosphere at room temperature for 4 hours. The mixture was filtered through a pad of Celite. The filtrate was concentrated to afford the desired product, ethyl 3-[3-[l-[10-[4,6-difluoro-1-(2-trimethylsilylethoxymethyl)indol-5-yl]oxy-6,7-dihydro-5H-imidazo[2,1-a][2]benzazepin-2-yl]ethyl]-2-fluoro-phenyl]propanoate (70 mg, 0.10 mmol, 87%) as a colorless oil. MS (ESI): 704 n/z (M+H)⁺.

### 3-[3-[1-[10-[(4,6-Difluoro-1H-indol-5-yl)oxy]-6,7-dihydro-5H-imidazo[2,1-a][2]benzazepin-2-yl]ethyl]-2-fluoro-phenyl]propanoic acid

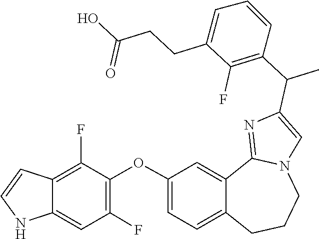 [SMILES: CC(c1cn2c(n1)-c1cc(Oc3c(F)cc4[nH]ccc4c3F)ccc1CCC2)c1cccc(CCC(=O)O)c1F]

Step P: To a stirred solution of ethyl 3-[3-[l-[10-[4,6-difluoro-1-(2-trimethylsilylethoxymethyl)indol-5-yl]oxy-6,7-dihydro-5H-imidazo[2,1-a][2]benzazepin-2-yl]ethyl]-2-fluoro-phenyl]propanoate (1.00 eq, 70 mg, 0.10 mmol) in TBAF (1M in THF, 5 mL) was added ethylenediamine (1.50 eq, 9.0 mg, 0.15 mmol). The mixture was stirred at 70° C. for 16 hours, and diluted with EtOAc (30 mL). The organic layer was washed with hydrochloric acid (1M, 20 mL×2), water (30 mL), brine (20 mL), dried over Na₂SO₄, and concentrated. The residue was purified by silica gel column chromatography, eluting with 0-100% MeOH in DCM, to afford the desired product, 3-[3-[1-[10-[(4,6-difluoro-1H-indol-5-yl)oxy]-6,7-dihydro-5H-imidazo[2,1-a][2]benzazepin-2-yl]ethyl]-2-fluoro-phenyl]propanoic acid (6.7 mg, 0.01 mmol, 12%). MS (ESI): 546 m/z (M+H)⁺. ¹H NMR (400 MHz, CD₃OD): δ 7.27 (t, J=5.6 Hz, 2H), 7.15-7.03 (m, 4H), 7.01-6.90 (m, 3H), 6.52 (d, J=3.1 Hz, 1H), 4.40 (q, J=6.4 Hz, 1H), 3.92 (t, J=6.9 Hz, 2H), 2.93 (t, J=7.6 Hz, 2H), 2.65 (t, J=7.0 Hz, 2H), 2.55 (t, J=7.6 Hz, 2H), 2.35-2.26 (m, 2H), 1.56 (d, J=7.2 Hz, 3H) ppm.

### Example 9. Synthesis of 3-(3-(1-(10-((4,6-difluoro-1H-indol-5-yl)oxy)-6,7-dihydro-5H-benzo[c][1,2,4]triazolo[1,5-a]azepin-2-yl)ethyl)-2-fluorophenyl)propanoic acid

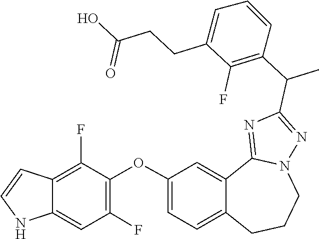 [SMILES: CC(c1nc2n(n1)CCCc1ccc(Oc3c(F)cc4[nH]ccc4c3F)cc1-2)c1cccc(CCC(=O)O)c1F]

### Tert-Butyl l-allyl-2-(2-(3-(3-ethoxy-3-oxopropyl)-2-fluorophenyl)propanoyl)hydrazine-1-carboxylate

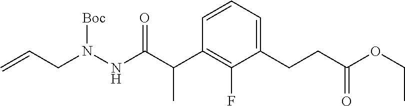 [SMILES: C=CCN(NC(=O)C(C)c1cccc(CCC(=O)OCC)c1F)C(=O)OC(C)(C)C]

Step A: To a stirred solution of 2-[3-(3-ethoxy-3-oxo-propyl)-2-fluoro-phenyl]propanoic acid (1.00 eq, 1.00 g, 3.73 mmol) in acetonitrile (60 mL) was added tert-butyl N-allyl-N-amino-carbamate (1.00 eq, 642 mg, 3.73 mmol), Chloro-N,N,N′,N′-tetramethylformamidinium hexafluorophosphate (1.00 eq, 1046 mg, 3.73 mmol) and 1-methylimidazole (3.50 eq, 1.0 mL, 13.0 mmol). The mixture was stirred at room temperature for 4 hours, quenched with water (60 mL) and extracted with EtOAc (70 mL×2). The organic layer was washed with water (70 mL×2), brine (80 mL), dried over Na₂SO₄, and concentrated. The residue was purified by silica gel column chromatography, eluting with 0-50% EtOAc in petroleum ether, to afford the desired product, ethyl 3-[3-[2-(2-allyl-2-tert-butoxycarbonyl-hydrazino)-1-methyl-2-oxo-ethyl]-2-fluoro-phenyl]propanoate (1.30 g, 3.08 mmol, 83%) as a white solid. MS (ESI): 445 m/z (M+Na)⁺.

### Ethyl 3-(3-(1-(2-allylhydrazineyl)-1-oxopropan-2-yl)-2-fluorophenyl)propanoate hydrochloride

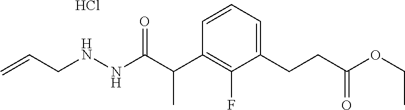 [SMILES: C=CCNNC(=O)C(C)c1cccc(CCC(=O)OCC)c1F ; Cl]

Step B: To a solution of ethyl 3-[3-[2-(2-allyl-2-tert-butoxycarbonyl-hydrazino)-1-methyl-2-oxo-ethyl]-2-fluoro-phenyl]propanoate (1.00 eq, 1.30 g, 3.08 mmol) was added 4M HCl solution in 1,4-dioxane (50 mL). The mixture was stirred at room temperature for 2 hours and concentrated to afford the desired product, ethyl 3-[3-[2-(2-allylhydrazino)-1-methyl-2-oxo-ethyl]-2-fluoro-phenyl]propanoate hydrochloride (1.20 g, 3.04 mmol, 99%) as a white solid. MS (ESI): 323 m/z (M+H)⁺.

### 5-((4,6-Difluoro-1-((2-(trimethylsilyl)ethoxy)methyl)-1H-indol-5-yl)oxy)-2-(methoxymethoxy)benzonitrile

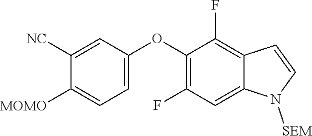

Step C: To a stirred solution of 5-[4,6-difluoro-1-(2-trimethylsilylethoxymethyl)indol-5-yl]oxy-2-hydroxy-benzonitrile (1.00 eq, 500 mg, 1.20 mmol) in DMF (6 mL) was added bromo(methoxy)methane (1.10 eq, 165 mg, 1.32 mmol). The mixture was cooled to 0° C. before NaH (1.20 eq, 35 mg, 1.44 mmol) was added. The mixture was warmed to room temperature and stirred for 16 hours, diluted with water (50 mL) and extracted with EtOAc (40 mL×2). The organic layer was washed with saturated aqueous LiCl (40 mL×2) and brine (30 mL), dried over Na₂SO₄ and concentrated to afford the desired product, 5-[4,6-difluoro-1-(2-trimethylsilylethoxymethyl)indol-5-yl]oxy-2-(methoxymethoxy)benzonitrile (600 mg, 0.78 mmol, 65%). MS (ESI): 461 m/z (M+H)⁺.

### 5-((4,6-Difluoro-1-((2-(trimethylsilyl)ethoxy)methyl)-1H-indol-5-yl)oxy)-2-(methoxymethoxy)benzothioamide

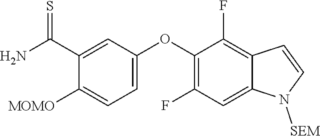

Step D: To a stirred solution of 5-[4,6-difluoro-1-(2-trimethylsilylethoxymethyl)indol-5-yl]oxy-2-(methoxymethoxy)benzonitrile (1.00 eq, 600 mg, 1.30 mmol) in DMF (8 mL) was added sodium hydrosulfide (6.00 eq, 454 mg, 7.82 mmol) and magnesium chloride (3.00 eq, 372 mg, 3.91 mmol). The mixture was stirred at room temperature for 16 hours, diluted with water, and extracted with EtOAc (50 mL×2). The organic layer was washed with saturated aqueous LiCl (50 mL×2), brine (50 mL), dried over Na₂SO₄, and concentrated to afford the desired product, 5-[4,6-difluoro-1-(2-trimethylsilylethoxymethyl)indol-5-yl]oxy-2-(methoxymethoxy)benzenecarbothioamide (600 mg, 0.91 mmol, 70%) as a brown oil. MS (ESI): 495 m/z (M+H)⁺.

### Methyl 5-((4,6-difluoro-1-((2-(trimethylsilyl)ethoxy)methyl)-1H-indol-5-yl)oxy)-2-(methoxymethoxy)benzimidothioate

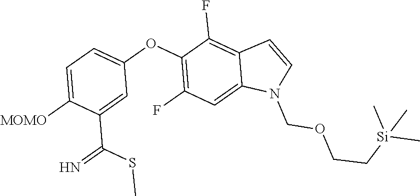

Step E: To a stirred solution of 5-[4,6-difluoro-1-(2-trimethylsilylethoxymethyl)indol-5-yl]oxy-2-(methoxymethoxy)benzenecarbothioamide (1.00 eq, 600 mg, 1.21 mmol) in acetone (10 mL) was added methyl iodide (1.00 eq, 0.076 mL, 1.21 mmol). The mixture was stirred at 50° C. for 16 hours and concentrated to afford the desired product, methyl 5-[4,6-difluoro-1-(2-trimethylsilylethoxymethyl)indol-5-yl]oxy-2-(methoxymethoxy)benzenecarboximidothioate (600 mg, 1.18 mmol, 97%) as a brown oil. MS (ESI): 509 m/z (M+H)⁺.

### Ethyl 3-(3-(1-(1-allyl-5-(5-((4,6-difluoro-1-((2-(trimethylsilyl)ethoxy)methyl)-1H-indol-5-yl)oxy)-2-hydroxyphenyl)-1H-1,2,4-triazol-3-yl)ethyl)-2-fluorophenyl)propanoate

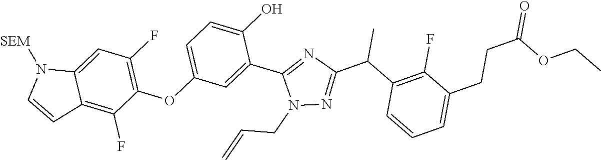 [SMILES: C=CCn1nc(C(C)c2cccc(CCC(=O)OCC)c2F)nc1-c1cc(Oc2c(F)cc3c(ccn3COCC[Si](C)(C)C)c2F)ccc1O]

Step F: To a stirred solution of ethyl 3-[3-[2-(2-allylhydrazino)-1-methyl-2-oxo-ethyl]-2-fluoro-phenyl]propanoate (1.00 eq, 347 mg, 1.08 mmol) in pyridine (10 mL) was added methyl 5-[4,6-difluoro-1-(2-trimethylsilylethoxymethyl)indol-5-yl]oxy-2-hydroxy-benzenecarboximidothioate (1.00 eq, 500 mg, 1.08 mmol). The mixture was stirred at 90° C. for 16 hours, cooled, and extracted with EtOAc (50 mL×2). The organic layer was washed with 1M hydrochloric acid (30 mL×2), brine (30 mL), dried over Na₂SO₄, and concentrated. The residue was purified by silica gel column chromatography, eluting with 0-30% EtOAc in petroleum ether, to afford the desired product, ethyl 3-[3-[1-[I-allyl-5-[5-[4,6-difluoro-1-(2-trimethylsilylethoxymethyl)indol-5-yl]oxy-2-hydroxy-phenyl]-1,2,4-triazol-3-yl]ethyl]-2-fluoro-phenyl]propanoate (100 mg, 0.14 mmol, 13%) as a brown solid. MS (ESI): 721 m/z (M+H)⁺.

### Ethyl 3-(3-(1-(1-allyl-5-(5-((4,6-difluoro-1-((2-(trimethylsilyl)ethoxy)methyl)-1H-indol-5-yl)oxy)-2-(((trifluoromethyl)sulfonyl)oxy)phenyl)-1H-1,2,4-triazol-3-yl)ethyl)-2-fluorophenyl)propanoate

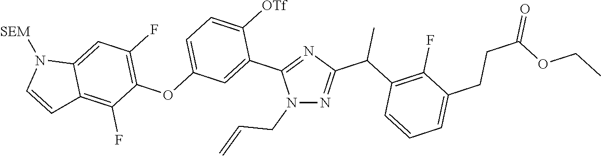 [SMILES: C=CCn1nc(C(C)c2cccc(CCC(=O)OCC)c2F)nc1-c1cc(Oc2c(F)cc3c(ccn3COCC[Si](C)(C)C)c2F)ccc1OS(=O)(=O)C(F)(F)F]

Step G: To a solution of ethyl 3-[3-[1-[1-allyl-5-[5-[4,6-difluoro-1-(2-trimethylsilylethoxymethyl)indol-5-yl]oxy-2-hydroxy-phenyl]-1,2,4-triazol-3-yl]ethyl]-2-fluoro-phenyl]propanoate (1.00 eq, 200 mg, 0.28 mmol) stirred in DCM (20 mL) was added DIPEA (3.00 eq, 107 mg, 0.83 mmol). The mixture was cooled to 0° C. before trifluoromethanesulfonic anhydride (1.50 eq, 0.070 mL, 0.42 mmol) was added. The reaction was stirred at 0° C. for 6 hours before quenching with saturated aqueous NaHCO₃ (20 mL). The organic layer was washed with brine (20 mL), dried over Na₂SO₄, and concentrated. The residue was purified by column chromatography on silica to afford the desired product, ethyl 3-[3-[1-[1-allyl-5-[5-[4,6-difluoro-1-(2-trimethylsilylethoxymethyl)indol-5-yl]oxy-2-(trifluoromethylsulfonyloxy)phenyl]-1,2,4-triazol-3-yl]ethyl]-2-fluoro-phenyl]propanoate (200 mg, 0.234 mmol, 85%) as a white solid. MS (ESI): 853 m/z (M+H)⁺.

### Ethyl 3-(3-(1-(1-allyl-5-(5-((4,6-difluoro-1-((2-(trimethylsilyl)ethoxy)methyl)-1H-indol-5-yl)oxy)-2-vinylphenyl)-1H-1,2,4-triazol-3-yl)ethyl)-2-fluorophenyl)propanoate

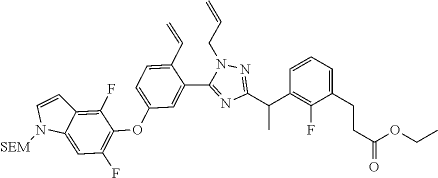 [SMILES: C=CCn1nc(C(C)c2cccc(CCC(=O)OCC)c2F)nc1-c1cc(Oc2c(F)cc3c(ccn3COCC[Si](C)(C)C)c2F)ccc1C=C]

Step H: To a stirred solution of ethyl 3-[3-[1-[1-allyl-5-[5-[4,6-difluoro-1-(2-trimethylsilylethoxymethyl)indol-5-yl]oxy-2-(trifluoromethylsulfonyloxy)phenyl]-1,2,4-triazol-3-yl]ethyl]-2-fluoro-phenyl]propanoate (1.00 eq, 180 mg, 0.21 mmol) in 1,4-Dioxane (9 mL) and water (1 mL) was added potassium carbonate (3.00 eq, 89 mg, 0.63 mmol), Pd(dppf)Cl₂ (0.10 eq, 17 mg, 0.02 mmol) and potassium vinyl trifluoroborate (1.0 eq, 28 mg, 0.21 mmol). The mixture was stirred at 90° C. for 16 hours, and diluted with EtOAc (50 mL). The organic layer was washed with water (30 mL×2), brine, dried over Na₂SO₄, and concentrated. The residue was purified by silica gel column chromatography to afford the desired product, ethyl 3-[3-[1-[1-allyl-5-[5-[4,6-difluoro-1-(2-trimethylsilylethoxymethyl)indol-5-yl]oxy-2-vinyl-phenyl]-1,2,4-triazol-3-yl]ethyl]-2-fluoro-phenyl]propanoate (100 mg, 0.137 mmol, 65%) as a colorless oil. MS (ESI): 731 m/z (M+H)⁺.

### Ethyl 3-(3-(1-(10-((4,6-difluoro-1-((2-(trimethylsilyl)ethoxy)methyl)-1H-indol-5-yl)oxy)-5H-benzo[c][1,2,4]triazolo[1,5-a]azepin-2-yl)ethyl)-2-fluorophenyl)propanoate

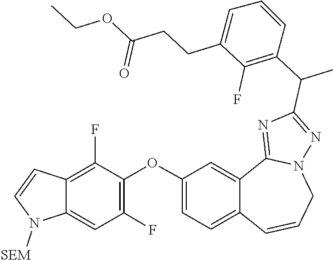 [SMILES: CCOC(=O)CCc1cccc(C(C)c2nc3n(n2)CC=Cc2ccc(Oc4c(F)cc5c(ccn5COCC[Si](C)(C)C)c4F)cc2-3)c1F]

Step I: To a stirred solution of ethyl 3-[3-[1-[1-allyl-5-[5-[4,6-difluoro-1-(2-trimethylsilylethoxymethyl)indol-5-yl]oxy-2-vinyl-phenyl]-1,2,4-triazol-3-yl]ethyl]-2-fluoro-phenyl]propanoate (1.00 eq, 30 mg, 0.04 mmol) in DCE (30 mL) was added 2^{nd} generation Grubbs catalysts (0.50 eq, 17 mg, 0.021 mmol). The mixture was stirred at 90° C. for 16 hours and concentrated. The residue was purified by silica gel column chromatography, eluting with 0-30% EtOAc in petroleum ether, to afford the desired product, ethyl 3-[3-[1-[10-[4,6-difluoro-1-(2-trimethylsilylethoxymethyl)indol-5-yl]oxy-5H-[1,2,4]triazolo[5,1-a][2]benzazepin-2-yl]ethyl]-2-fluoro-phenyl]propanoate (15 mg, 0.02 mmol, 52%) as a colorless oil. MS (ESI): 703 m/z (M+H)⁺.

### Ethyl 3-(3-(1-(10-((4,6-difluoro-1-((2-(trimethylsilyl)ethoxy)methyl)-1H-indol-5-yl)oxy)-6,7-dihydro-5H-benzo[c][1,2,4]triazolo[1,5-a]azepin-2-yl)ethyl)-2-fluorophenyl)propanoate

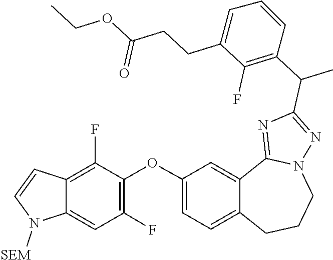 [SMILES: CCOC(=O)CCc1cccc(C(C)c2nc3n(n2)CCCc2ccc(Oc4c(F)cc5c(ccn5COCC[Si](C)(C)C)c4F)cc2-3)c1F]

Step J: To a stirred solution of ethyl 3-[3-[1-[10-[4,6-difluoro-1-(2-trimethylsilylethoxymethyl)indol-5-yl]oxy-5H-[1,2,4]triazolo[5,1-a][2]benzazepin-2-yl]ethyl]-2-fluoro-phenyl]propanoate (1.00 eq, 60 mg, 0.085 mmol) in ethanol (10 mL) was added Pd on activated carbon (10 wt. %, 50 mg). The mixture was stirred at room temperature for 16 hours under a hydrogen atmosphere, filtered, and concentrated to afford the desired product, ethyl 3-[3-[1-[10-[4,6-difluoro-1-(2-trimethylsilylethoxymethyl)indol-5-yl]oxy-6,7-dihydro-5H-[1,2,4]triazolo[5,1-a][2]benzazepin-2-yl]ethyl]-2-fluoro-phenyl]propanoate (55 mg, 0.08 mmol, 91%) as colorless oil. MS (ESI): 705 m/z (M+H)⁺.

### 3-(3-(1-(10-((4,6-Difluoro-1H-indol-5-yl)oxy)-6,7-dihydro-5H-benzo[c][1,2,4]triazolo[1,5-a]azepin-2-yl)ethyl)-2-fluorophenyl)propanoic acid

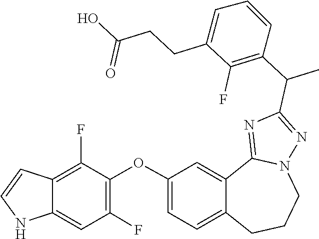 [SMILES: CC(c1nc2n(n1)CCCc1ccc(Oc3c(F)cc4[nH]ccc4c3F)cc1-2)c1cccc(CCC(=O)O)c1F]

Step K: To a stirred solution of ethyl 3-[3-[1-[10-[4,6-difluoro-1-(2-trimethylsilylethoxymethyl)indol-5-yl]oxy-6,7-dihydro-5H-[1,2,4]triazolo[5,1-a][2]benzazepin-2-yl]ethyl]-2-fluoro-phenyl]propanoate (1.00 eq, 10 mg, 0.014 mmol) in methanol (1 mL) was added 4M HCl in methanol (2 mL). The mixture was stirred at room temperature for 16 hours and concentrated. To the residue was added, THF (2 mL), a solution of LiOH (5 mg) in water (2 ml). The mixture was stirred at room temperature for another 16 hours. Purification of the crude product by prep-HPLC afforded the desired product, 3-[3-[1-[10-[(4,6-difluoro-1H-indol-5-yl)oxy]-6,7-dihydro-5H-[1,2,4]triazolo[5,1-a][2]benzazepin-2-yl]ethyl]-2-fluoro-phenyl]propanoic acid (0.57 mg, 0.00104 mmol, 7%). MS (ESI): 547 m/z (M+H)⁺. ¹H NMR (400 MHz, CD₃OD): δ 7.38 (d, J=2.5 Hz, 1H), 7.33-7.26 (m, 2H), 7.11 (d, J=9.1 Hz, 3H), 6.98 (t, J=7.5 Hz, 2H), 6.53 (d, J=2.9 Hz, 1H), 4.55 (q, J=7.1 Hz, 1H), 4.26 (t, J=6.8 Hz, 2H), 2.93 (s, 2H), 2.80 (t, J=6.6 Hz, 2H), 2.56 (s, 2H), 2.38 (dt, J=13.2, 6.6 Hz, 2H), 1.63 (d, J=7.2 Hz, 4H) ppm.

### Example 10

### Synthesis of methyl 3-(3-(1-(11-((4,6-difluoro-1H-indol-5-yl)oxy)-6,7-dihydro-5H-benzo[b][1,2,4]triazolo[5,1-d][1,5]oxazocin-2-yl)ethyl)-2-fluorophenyl)propanoate

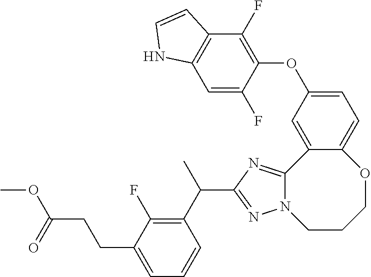 [SMILES: COC(=O)CCc1cccc(C(C)c2nc3n(n2)CCCOc2ccc(Oc4c(F)cc5[nH]ccc5c4F)cc2-3)c1F]

### 2-[3-(3-Ethoxy-3-oxo-propyl)-2-fluoro-phenyl]propanoic acid

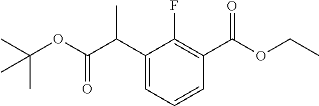 [SMILES: CCOC(=O)c1cccc(C(C)C(=O)OC(C)(C)C)c1F]

Step A: To a solution of tert-butyl 2-[3-(3-ethoxy-3-oxo-propyl)-2-fluoro-phenyl]propanoate (1.00 eq, 6.00 g, 18.5 mmol) in DCM (20 mL) was added trifluoroacetic acid (14.0 eq, 20 mL, 260 mmol). The reaction was stirred at room temperature for 2 h. The reaction mixture was concentrated and purified by flash column chromatography on silica gel, eluting with 0-15% ethyl acetate in petroleum ether, to give 2-[3-(3-ethoxy-3-oxo-propyl)-2-fluoro-phenyl]propanoic acid (4.70 g, 15.6 mmol, 84% yield) as a colorless oil. MS (ESI): 269 m/z (M+H)⁺.

### Ethyl 3-[3-[2-(2-allyl-2-tert-butoxycarbonyl-hydrazino)-1-methyl-2-oxo-ethyl]-2-fluoro-phenyl]propanoate

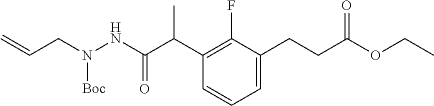 [SMILES: C=CCN(NC(=O)C(C)c1cccc(CCC(=O)OCC)c1F)C(=O)OC(C)(C)C]

Step B: To a solution of 2-[3-(3-ethoxy-3-oxo-propyl)-2-fluoro-phenyl]propanoic acid (1.00 eq, 13.90 g, 51.8 mmol) in MeCN (130 mL) was added 1-methylimidazole (4.50 eq, 19 mL, 233 mmol), tert-butyl N-allyl-N-amino-carbamate (1.10 eq, 9.82 g, 57.0 mmol) and chloro-N,N,N′,N′-tetramethylformamidinium hexafluorophosphate (1.05 eq, 15.26 g, 54.4 mmol). The reaction was stirred at room temperature for 2 hours. The reaction mixture was diluted with water (200 mL). The product was extracted with ethyl acetate (200 mL). The separated organic layer was washed with brine (2×100 mL), dried over anhydrous sodium sulfate, filtered, and concentrated. The residue was purified by flash column chromatography on silica gel, eluting with 0-15% ethyl acetate in petroleum ether, to give ethyl 3-[3-[2-(2-allyl-2-tert-butoxycarbonyl-hydrazino)-1-methyl-2-oxo-ethyl]-2-fluoro-phenyl]propanoate (20.00 g, 45.4 mmol, 88% yield) as an oil. MS (ESI): 445 m/z (M+Na)⁺.

### Ethyl 3-[3-[2-(2-allylhydrazino)-1-methyl-2-oxo-ethyl]-2-fluoro-phenyl]propanoate

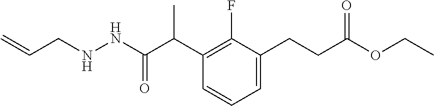 [SMILES: C=CCNNC(=O)C(C)c1cccc(CCC(=O)OCC)c1F]

Step C: To a solution of ethyl 3-[3-[2-(2-allyl-2-tert-butoxycarbonyl-hydrazino)-1-methyl-2-oxo-ethyl]-2-fluoro-phenyl]propanoate (1.00 eq, 19.00 g, 45.0 mmol) in DCM (100 mL) was added HCl (4M in dioxane, 100 mL). The reaction was stirred at room temperature for 2 hours. The mixture was quenched with 1M potassium carbonate aqueous solution (150 mL), extracted with ethyl acetate (150 mL). The organic extract was washed with brine (2×100 mL), dried over anhydrous sodium sulfate, filtered, and concentrated. The residue was purified by flash chromatography on silica, eluting with 0-35% methanol in dichloromethane, to give ethyl 3-[3-[2-(2-allylhydrazino)-1-methyl-2-oxo-ethyl]-2-fluoro-phenyl]propanoate (13.50 g, 39.8 mmol, 88% yield). MS (ESI): 323 m/z (M+H)⁺.

### 5-[4,6-Difluoro-1-(2-trimethylsilylethoxymethyl)indol-5-yl]oxy-2-fluoro-benzonitrile

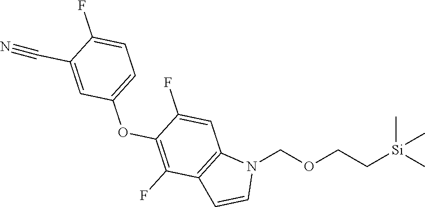 [SMILES: C[Si](C)(C)CCOCn1ccc2c(F)c(Oc3ccc(F)c(C#N)c3)c(F)cc21]

Step D: To a solution of 5-[(4,6-difluoro-1H-indol-5-yl)oxy]-2-fluoro-benzonitrile (1.00 eq, 10.00 g, 34.7 mmol) in DMF (100 mL) at 0° C. under N2 was added sodium hydride (1.20 eq, 999 mg, 41.6 mmol). The mixture was stirred for 1 hour, treated with 2-(trimethylsilyl)ethoxymethyl chloride (1.10 eq, 6.8 mL, 38.2 mmol), and stirred at 0° C. for another 2 hours. The mixture was diluted with water (300 mL) and extracted with ethyl acetate (100 mL×3). The combined organic extracts were washed with brine (50 mL×2), dried over anhydrous sodium sulfate, filtered, and concentrated. The residue was purified by flash chromatography on silica, eluting with 0-15% ethyl acetate in petroleum ether, to give 5-[4,6-difluoro-1-(2-trimethylsilylethoxymethyl)indol-5-yl]oxy-2-fluoro-benzonitrile (12.00 g, 28.7 mmol, 82.65% yield). MS (ESI): 419 m/z (M+H)⁺.

### 5-[4,6-Difluoro-1-(2-trimethylsilylethoxynethyl)indol-5-yl]oxy-2-hydroxy-benzonitrile

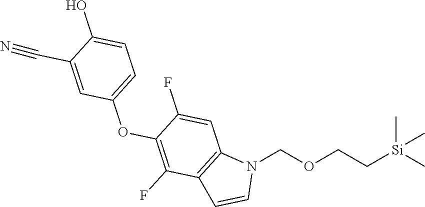 [SMILES: C[Si](C)(C)CCOCn1ccc2c(F)c(Oc3ccc(O)c(C#N)c3)c(F)cc21]

Step E: To a stirred solution of 5-[4,6-difluoro-1-(2-trimethylsilylethoxymethyl)indol-5-yl]oxy-2-fluoro-benzonitrile (1.00 eq, 12.00 g, 28.7 mmol) in DMF (180 mL) was added sodium hydride (3.00 eq, 2065 mg, 86.0 mmol) at 0° C. under N2. The mixture was stirred at 0° C. for 1 hour, treated with 2-(methylsulfonyl)ethanol (1.50 eq, 5340 mg, 43.0 mmol), and stirred at 0° C. for another 16 hours. The mixture was diluted with water (300 mL) and extracted with ethyl acetate (100 mL×3). The combined organic extracts were washed with brine (50 mL×2), dried over anhydrous sodium sulfate, filtered, and concentrated. The residue was purified by flash chromatography on silica, eluting with 0-15% ethyl acetate in petroleum ether, to give 5-[4,6-difluoro-1-(2-trimethylsilylethoxymethyl)indol-5-yl]oxy-2-hydroxy-benzonitrile (8.00 g, 19.2 mmol, 67% yield). MS (ESI): 417 m/z (M+H)⁺.

### 5-[4,6-Difluoro-1-(2-trimethylsilylethoxymethyl)indol-5-yl]oxy-2-(methoxymethoxy)benzonitrile

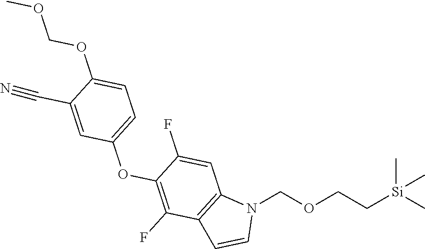 [SMILES: COCOc1ccc(Oc2c(F)cc3c(ccn3COCC[Si](C)(C)C)c2F)cc1C#N]

Step F: To a stirred solution of 5-[4,6-difluoro-1-(2-trimethylsilylethoxymethyl)indol-5-yl]oxy-2-hydroxy-benzonitrile (1.00 eq, 8.00 g, 19.2 mmol) in DMF (100 mL) was added sodium hydride (1.30 eq, 599 mg, 25.0 mmol) at 0° C. under N2. After stirring for 1 hour, bromo(methoxy)methane (1.20 eq, 2880 mg, 23.0 mmol) was added. The reaction was stirred at 0° C. for 16 hours. The mixture was diluted with water (300 mL) and extracted with ethyl acetate (100 mL×3). The combined extracts were washed with brine (50 mL×2), dried over anhydrous sodium sulfate, filtered, and concentrated. The residue was purified by flash chromatography on silica, eluting with 0-23% ethyl acetate in petroleum ether, to give 5-[4,6-difluoro-1-(2-trimethylsilylethoxymethyl)indol-5-yl]oxy-2-(methoxymethoxy)benzonitrile (7.00 g, 15.2 mmol, 79% yield). MS (ESI): 461 m/z (M+H)⁺.

### 5-[4,6-Difluoro-1-(2-trimethylsilylethoxymethyl)indol-5-yl]oxy-2-(methoxymethoxy) benzenecarbothioamide

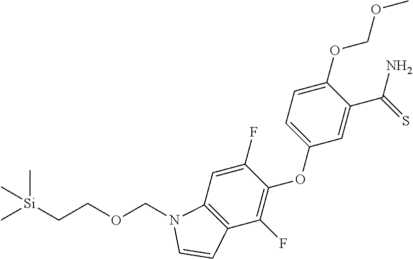

Step G: To a solution of magnesium chloride hexahydrate (3.00 eq, 2384 mg, 11.7 mmol) in DMF (20 mL) was added sodium bisulfide (6.00 eq, 1315 mg, 23.5 mmol) and 5-[4,6-difluoro-1-(2-trimethylsilylethoxymethyl)indol-5-yl]oxy-2-(methoxymethoxy)benzonitrile (1.00 eq, 1.80 g, 3.91 mmol). The mixture was stirred at room temperature for 16 hours, diluted with water (100 mL), and extracted with ethyl acetate (50 mL×3). The combined organic extracts were washed with brine (30 mL), dried over anhydrous sodium sulfate, filtered, and concentrated. The residue was purified by flash chromatography on silica gel, eluting with 0-30% ethyl acetate in petroleum ether, to give 5-[4,6-difluoro-1-(2-trimethylsilylethoxymethyl)indol-5-yl]oxy-2-(methoxymethoxy)benzenecarbothioamide (1.80 g, 3.64 mmol, 93% yield). MS (ESI): 495 m/z (M+H)⁺

### Methyl 5-[4,6-difluoro-1-(2-trimethylsilylethoxymethyl)indol-5-yl]oxy-2-(methoxymethoxy) benzenecarboximidothioate

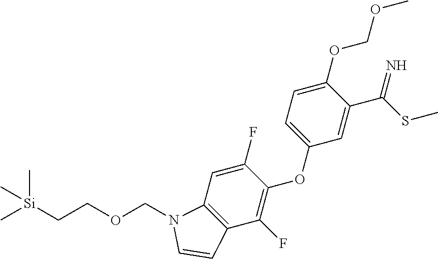

Step H: To a solution of 5-[4,6-difluoro-1-(2-trimethylsilylethoxymethyl)indol-5-yl]oxy-2-(methoxymethoxy)benzenecarbothioamide (1.00 eq, 1.80 g, 3.64 mmol) in acetone (40 mL) was added methyl iodide (5.00 eq, 1.1 mL, 18.2 mmol) and sodium bicarbonate (5.00 eq, 1529 mg, 18.2 mmol). The mixture was stirred at room temperature for 16 hours, diluted with water (100 mL), and extracted with ethyl acetate (50 mL×3). The combined organic extracts were washed with brine (30 mL), dried over anhydrous sodium sulfate, filtered, and concentrated to give methyl 5-[4,6-difluoro-1-(2-trimethylsilylethoxymethyl)indol-5-yl]oxy-2-(methoxymethoxy)benzenecarboximidothioate (1.80 g, 3.54 mmol, 97% yield). MS (ESI): 509.2 m/z (M+H)⁺

### Ethyl 3-[3-[1-[1-allyl-5-[5-[4,6-difluoro-1-(2-trimethylsilylethoxymethyl)indol-5-yl]oxy-2-(methoxymethoxy)phenyl]-1,2,4-triazol-3-yl]ethyl]-2-fluoro-phenyl]propanoate

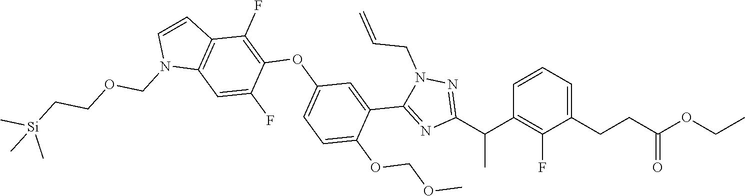 [SMILES: C=CCn1nc(C(C)c2cccc(CCC(=O)OCC)c2F)nc1-c1cc(Oc2c(F)cc3c(ccn3COCC[Si](C)(C)C)c2F)ccc1OCOC]

Step I: To a solution of methyl 5-[4,6-difluoro-1-(2-trimethylsilylethoxymethyl)indol-5-yl]oxy-2-(methoxymethoxy)benzenecarboximidothioate (1.00 eq, 3.00 g, 5.90 mmol) in pyridine (30 mL) was added ethyl 3-[3-[2-(2-allylhydrazino)-1-methyl-2-oxo-ethyl]-2-fluoro-phenyl]propanoate (1.20 eq, 2282 mg, 7.08 mmol). The reaction mixture was stirred at 90° C. for 3 days. The mixture was diluted with water (100 mL), extracted with ethyl acetate (50 mL×3), washed with brine (100 mL×2), dried over anhydrous sodium sulfate, filtered, and concentrated. The residue was purified by flash chromatography on silica, eluting with 0-19% ethyl acetate in petroleum ether, to give ethyl 3-[3-[1-[1-allyl-5-[5-[4,6-difluoro-1-(2-trimethylsilylethoxymethyl)indol-5-yl]oxy-2-(methoxymethoxy)phenyl]-1,2,4-triazol-3-yl]ethyl]-2-fluoro-phenyl]propanoate (520 mg, 0.680 mmol, 11% yield). MS (ESI): 765.3 m/z (M+H)⁺

### Ethyl 3-[3-[1-[5-[5-[4,6-difluoro-1-(2-trimethylsilylethoxynethyl)indol-5-yl]oxy-2-(methoxymethoxy)phenyl]-1-(3-hydroxypropyl)-1,2,4-triazol-3-yl]ethyl]-2-fluoro-phenyl]propanoate

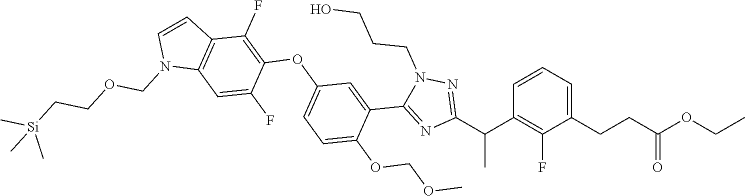 [SMILES: CCOC(=O)CCc1cccc(C(C)c2nc(-c3cc(Oc4c(F)cc5c(ccn5COCC[Si](C)(C)C)c4F)ccc3OCOC)n(CCCO)n2)c1F]

Step J: To a solution of ethyl 3-[3-[1-[1-allyl-5-[5-[4,6-difluoro-1-(2-trimethylsilylethoxymethyl) indol-5-yl]oxy-2-(methoxymethoxy)phenyl]-1,2,4-triazol-3-yl]ethyl]-2-fluoro-phenyl]propanoate (1.00 eq, 570 mg, 0.745 mmol) in THE (2 mL) was added 9-borabicyclo[3.3.1]nonane (1.00 eq, 10 mL, 0.745 mmol). The mixture was stirred at room temperature for 16 hours. After consumption of the starting material, hydrogen peroxide (219 eq, 5.0 mL, 163 mmol) and sodium carbonate (6.71 eq, 5.0 mL, 5.00 mmol, 1 M in water) were added. The mixture was stirred at room temperature for 4 hours, then diluted with water (50 mL), and extracted with ethyl acetate (30 mL×2). The combined extracts were washed with brine (20 mL), dried over anhydrous sodium sulfate, filtered, and concentrated. The residue was purified by flash chromatography on silica, eluting with 0-50% ethyl acetate in petroleum ether, to give ethyl 3-[3-[1-[5-[5-[4,6-difluoro-1-(2-trimethylsilylethoxymethyl)indol-5-yl]oxy-2-(methoxymethoxy)phenyl]-1-(3-hydroxypropyl)-1,2,4-triazol-3-yl]ethyl]-2-fluoro-phenyl]propanoate (190 mg, 0.243 mmol, 32% yield). MS (ESI): 783.3 m/z (M+H)⁺

### 3-[3-[1-[5-[5-[(4,6-Difluoro-1H-indol-5-yl)oxy]-2-(methoxymethoxy)phenyl]-1-(3-hydroxypropyl)-1,2,4-triazol-3-yl]ethyl]-2-fluoro-phenyl]propanoic acid

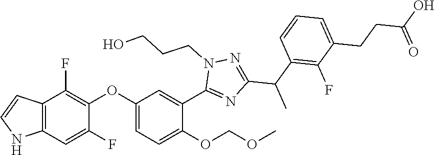 [SMILES: COCOc1ccc(Oc2c(F)cc3[nH]ccc3c2F)cc1-c1nc(C(C)c2cccc(CCC(=O)O)c2F)nn1CCCO]

Step K: To a solution of ethyl 3-[3-[1-[5-[5-[4,6-difluoro-1-(2-trimethylsilylethoxymethyl)indol-5-yl]oxy-2-(methoxymethoxy)phenyl]-1-(3-hydroxypropyl)-1,2,4-triazol-3-yl]ethyl]-2-fluoro-phenyl]propanoate (1.00 eq, 190 mg, 0.243 mmol) in TBAF (1.00 eq, 5.0 mL, 0.243 mmol) was added ethylenediamine (1.50 eq, 0.025 mL, 0.364 mmol). The mixture was stirred at 70° C. for 16 hours. To the mixture was added aqueous hydrochloric acid (1 M) to adjust the pH to 2, followed by extraction with ethyl acetate (50 mL×3). The combined organic extracts were washed with brine (20 mL), dried over anhydrous sodium sulfate, filtered and concentrated to afford 3-[3-[1-[5-[5-[(4,6-difluoro-1H-indol-5-yl)oxy]-2-(methoxymethoxy) phenyl]-1-(3-hydroxypropyl)-1,2,4-triazol-3-yl]ethyl]-2-fluoro-phenyl]propanoic acid (130 mg, 0.208 mmol, 86% yield). MS (ESI): 625.3 m/z (M+H)⁺

### Methyl 3-[3-[1-[5-[5-[(4,6-difluoro-1H-indol-5-yl)oxy]-2-hydroxy-phenyl]-1-(3-hydroxypropyl)-1,2,4-triazol-3-yl]ethyl]-2-fluoro-phenyl]propanoate

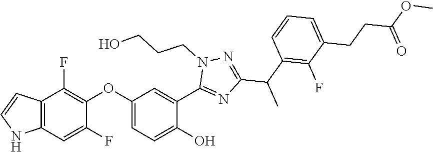 [SMILES: COC(=O)CCc1cccc(C(C)c2nc(-c3cc(Oc4c(F)cc5[nH]ccc5c4F)ccc3O)n(CCCO)n2)c1F]

Step L: To a solution of 3-[3-[1-[5-[5-[(4,6-difluoro-1H-indol-5-yl)oxy]-2-(methoxymethoxy)phenyl]-1-(3-hydroxypropyl)-1,2,4-triazol-3-yl]ethyl]-2-fluoro-phenyl]propanoic acid (1.00 eq, 130 mg, 0.208 mmol) in methanol (238 eq, 2.0 mL, 49.4 mmol) was added sulfuric acid (45.1 eq, 0.50 mL, 9.38 mmol). The mixture was stirred at 50° C. for 1 hour, diluted with water (100 mL), and extracted with ethyl acetate (50 mL×2). The combined organic extracts were washed with brine (20 mL×2), dried over anhydrous sodium sulfate, filtered, and concentrated. The residue was purified by flash chromatography on silica, eluting with 0-83% ethyl acetate in petroleum ether, to give methyl 3-[3-[1-[5-[5-[(4,6-difluoro-1H-indol-5-yl)oxy]-2-hydroxy-phenyl]-1-(3-hydroxypropyl)-1,2,4-triazol-3-yl]ethyl]-2-fluoro-phenyl]propanoate (100 mg, 0.168 mmol, 81% yield). MS (ESI): 595.2 m/z (M+H)⁺

### Methyl 3-[3-[1-[14-[(4,6-difluoro-1H-indol-5-yl)oxy]-10-oxa-3, 5,6-triazatricyclo[9.4.0.02,6]pentadeca-1(15),2,4,11,13-pentaen-4-yl]ethyl]-2-fluoro-phenyl]propanoate

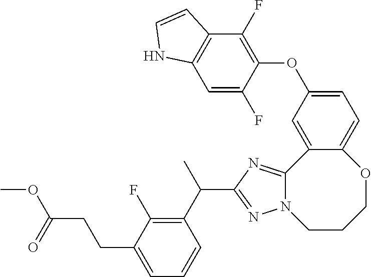 [SMILES: COC(=O)CCc1cccc(C(C)c2nc3n(n2)CCCOc2ccc(Oc4c(F)cc5[nH]ccc5c4F)cc2-3)c1F]

Step M: To a solution of methyl 3-[3-[1-[5-[5-[(4,6-difluoro-1H-indol-5-yl)oxy]-2-hydroxy-phenyl]-1-(3-hydroxypropyl)-1,2,4-triazol-3-yl]ethyl]-2-fluoro-phenyl]propanoate (1.00 eq, mg, 0.0252 mmol) in toluene (16 mL) was added under N2 (cyanomethylene)tributylphosphorane (6.00 eq, 37 mg, 0.151 mmol). The mixture was stirred at 150° C. in a microwave oven for 1 hour. The mixture was diluted with water (100 mL) and extracted with ethyl acetate (50 mL×2). The combined organic extracts were washed with brine (30 mL×2), dried over anhydrous sodium sulfate, filtered, and concentrated. The residue was purified by preparatory HPLC to give methyl 3-[3-[l-[14-[(4,6-difluoro-1H-indol-5-yl)oxy]-10-oxa-3,5,6-triazatricyclo[9.4.0.02,6]pentadeca-1(15),2,4,11,13-pentaen-4-yl]ethyl]-2-fluoro-phenyl]propanoate (10 mg, 0.0173 mmol, 69% yield). MS (ESI): 577.3 m/z (M+H)⁺

### Example 11

### Synthesis of 3-[3-[1-[14-[(4,6-difluoro-1H-indol-5-yl)oxy]-10-oxa-3,5,6-triazatricyclo[9.4.0.02,6]pentadeca-1(15),2,4,11,13-pentaen-4-yl]ethyl]-2-fluoro-phenyl]propanoic acid

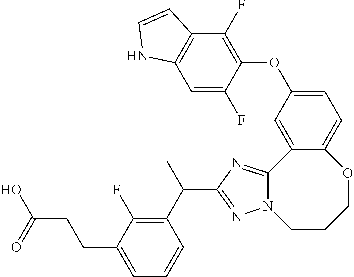 [SMILES: CC(c1nc2n(n1)CCCOc1ccc(Oc3c(F)cc4[nH]ccc4c3F)cc1-2)c1cccc(CCC(=O)O)c1F]

Step A: To a solution of methyl 3-[3-[1-[14-[(4,6-difluoro-1H-indol-5-yl)oxy]-10-oxa-3,5,6-triazatricyclo[9.4.0.02,6]pentadeca-1(15),2,4,11,13-pentaen-4-yl]ethyl]-2-fluoro-phenyl]propanoate (1.00 eq, 10 mg, 0.0173 mmol) in tetrahydrofuran (2 mL) was added lithium hydroxide (2.00 mL, 1M in water). The reaction was stirred at room temperature for 6 hours. The mixture was acidified with 1M hydrogen chloride in water (10 mL), extracted with ethyl acetate (30 mL). The organic extract was washed with brine (20 mL×2), dried over anhydrous sodium sulfate, filtered, and concentrated. The residue was purified by prep-HPLC to give 3-[3-[1-[14-[(4,6-difluoro-H-indol-5-yl)oxy]-10-oxa-3,5,6-triazatricyclo[9.4.0.02,6]pentadeca-1(15),2,4,11,13-pentaen-4-yl]ethyl]-2-fluoro-phenyl]propanoic acid (4.7 mg, 0.00835 mmol, 48% yield). MS (ESI): 563.2 m/z (M+H)⁺

### Example 12

### Synthesis of 3-[3-[1-[10-[(4,6-difluoro-1H-indol-5-yl)oxy]-6-(hydroxymethyl)-5,6-dihydro-[1,2,4]triazolo[1,5-d][1,4]benzoxazepin-2-yl]ethyl]-2-fluoro-phenyl]propanoic acid

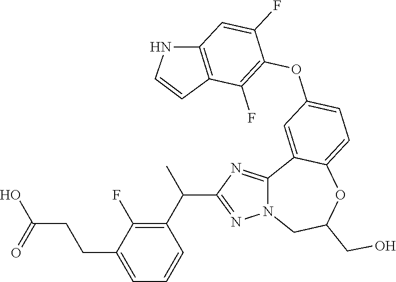 [SMILES: CC(c1nc2n(n1)CC(CO)Oc1ccc(Oc3c(F)cc4[nH]ccc4c3F)cc1-2)c1cccc(CCC(=O)O)c1F]

### Ethyl 3-[3-[1-[1-allyl-5-[5-[4,6-difluoro-1-(2-trimethylsilylethoxymethyl)indol-5-yl]oxy-2-(methoxymethoxy)phenyl]-1,2,4-triazol-3-yl]ethyl]-2-fluoro-phenyl]propanoate

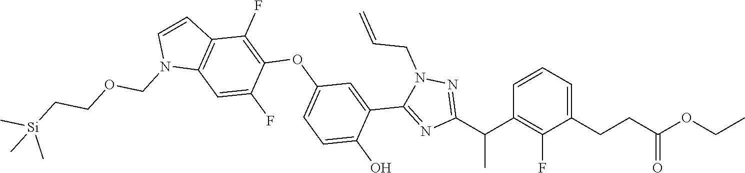 [SMILES: C=CCn1nc(C(C)c2cccc(CCC(=O)OCC)c2F)nc1-c1cc(Oc2c(F)cc3c(ccn3COCC[Si](C)(C)C)c2F)ccc1O]

Step A: To a solution of methyl 5-[4,6-difluoro-1-(2-trimethylsilylethoxymethyl)indol-5-yl]oxy-2-(methoxymethoxy)benzenecarboximidothioate (1.00 eq, 1.80 g, 3.54 mmol) in pyridine (18 mL) was added ethyl 3-[3-[2-(2-allylhydrazino)-1-methyl-2-oxo-ethyl]-2-fluoro-phenyl]propanoate (1.30 eq, 1483 mg, 4.60 mmol). The mixture was stirred at 90° C. for 16 hours, diluted with water (150 mL), and extracted with ethyl acetate (50 mL×3). The combined organic extracts were washed with brine (50 mL×2), dried over anhydrous sodium sulfate, filtered, and concentrated. The residue was purified by flash chromatography on silica, eluting with 0-23% ethyl acetate in petroleum ether, to give ethyl 3-[3-[1-[1-allyl-5-[5-[4,6-difluoro-1-(2-trimethylsilylethoxymethyl)indol-5-yl]oxy-2-(methoxymethoxy)phenyl]-1,2,4-triazol-3-yl]ethyl]-2-fluoro-phenyl]propanoate (1.40 g, 1.83 mmol, 52% yield)—product A, and ethyl 3-[3-[1-[I-allyl-5-[5-[4,6-difluoro-1-(2-trimethylsilylethoxymethyl)indol-5-yl]oxy-2-hydroxy-phenyl]-1,2,4-triazol-3-yl]ethyl]-2-fluoro-phenyl]propanoate (730 mg, 1.01 mmol, 29% yield)—product B.
Product A: MS (ESI): 765.3 m/z (M+H)⁺
Product B: MS (ESI): 721.3 m/z (M+H)⁺

### Ethyl 3-[3-[1-[5-[5-[4,6-difluoro-1-(2-trimethylsilylethoxymethyl)indol-5-yl]oxy-2-hydroxy-phenyl]-1-(2,3-dihydroxypropyl)-1,2,4-triazol-3-yl]ethyl]-2-fluoro-phenyl]propanoate

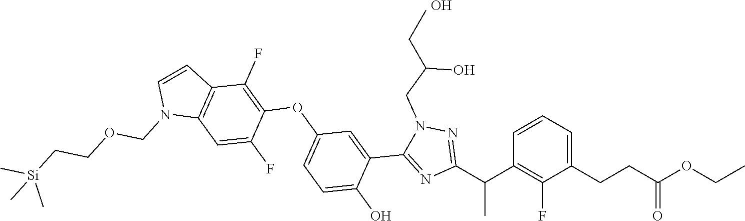

Step B: To a stirred solution of ethyl 3-[3-[1-[I-allyl-5-[5-[4,6-difluoro-1-(2-trimethylsilylethoxymethyl)indol-5-yl]oxy-2-hydroxy-phenyl]-1,2,4-triazol-3-yl]ethyl]-2-fluoro-phenyl]propanoate (1.00 eq, 100 mg, 0.139 mmol) in THE (2 mL) and water (0.4 mL) was added potassium osmate (VI) dihydrate (0.0500 eq, 2.6 mg, 0.00694 mmol) and sodium periodate (3.00 eq, 89 mg, 0.416 mmol). The mixture was stirred at room temperature for 16 hours, diluted with water (100 mL) and extracted with ethyl acetate (50 mL×2). The combined organic extracts were washed with brine (30 mL×2), dried over anhydrous sodium sulfate, filtered, and concentrated. The residue was purified by flash chromatography on silica gel, eluting with 0-59% ethyl acetate in petroleum ether, to give ethyl 3-[3-[1-[5-[5-[4,6-difluoro-1-(2-trimethylsilylethoxymethyl)indol-5-yl]oxy-2-hydroxy-phenyl]-1-(2,3-dihydroxypropyl)-1,2,4-triazol-3-yl]ethyl]-2-fluoro-phenyl]propanoate (62 mg, 0.0821 mmol, 59% yield). MS (ESI): 755.2 m/z (M+H)⁺

### Ethyl 3-[3-[1-[10-[4,6-difluoro-1-(2-trimethylsilylethoxymethyl)indol-5-yl]oxy-6-(hydroxymethyl)-5,6-dihydro-[1,2,4]triazolo[1,5-d][1,4]benzoxazepin-2-yl]ethyl]-2-fluoro-phenyl]propanoate and ethyl 3-[3-[1-[14-[4,6-difluoro-1-(2-trimethylsilylethoxymethyl)indol-5-yl]oxy-8-hydroxy-10-oxa-3,5,6-triazatricyclo[9.4.0.02,6]pentadeca-1(15),2,4,11,13-pentaen-4-yl]ethyl]-2-fluoro-phenyl]propanoate

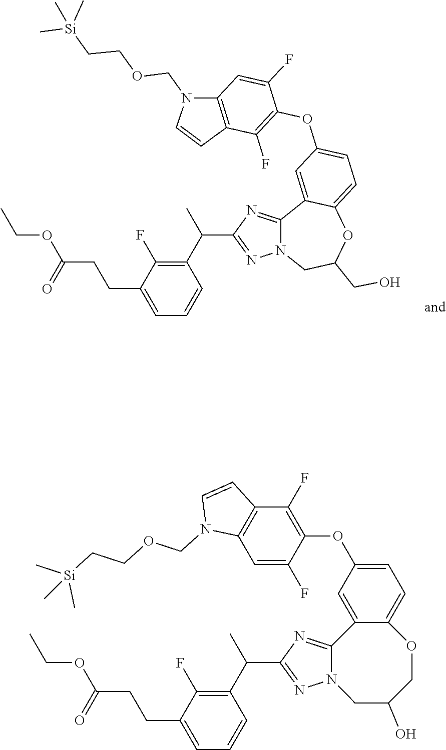 [SMILES: CCOC(=O)CCc1cccc(C(C)c2nc3n(n2)CC(CO)Oc2ccc(Oc4c(F)cc5c(ccn5COCC[Si](C)(C)C)c4F)cc2-3)c1F ; CCOC(=O)CCc1cccc(C(C)c2nc3n(n2)CC(O)COc2ccc(Oc4c(F)cc5c(ccn5COCC[Si](C)(C)C)c4F)cc2-3)c1F]

Step C: To a stirred solution of ethyl 3-[3-[1-[5-[5-[4,6-difluoro-1-(2-trimethylsilylethoxymethyl)indol-5-yl]oxy-2-hydroxy-phenyl]-1-(2,3-dihydroxypropyl)-1,2,4-triazol-3-yl]ethyl]-2-fluoro-phenyl]propanoate (1.00 eq, 120 mg, 0.159 mmol) in toluene (100 mL) was added under N2 cyanomethylenetributylphosphorane (6.00 eq, 230 mg, 0.95 mmol). The mixture was stirred at 150° C. in a microwave oven for 1 hour. The mixture was diluted with water (200 mL) and extracted with ethyl acetate (50 mL×2). The combined organic extracts were washed with brine (30 mL×2), dried over anhydrous sodium sulfate, filtered, and concentrated. The residue was purified by prep-HPLC to give ethyl 3-[3-[1-[(8Z)-14-[4,6-difluoro-1-(2-trimethylsilylethoxymethyl)indol-5-yl]oxy-10-oxa-3,5,6-triazatricyclo[9.4.0.02,6]pentadeca-1(15),2,4,8,11,13-hexaen-4-yl]ethyl]-2-fluoro-phenyl]propanoate (15 mg, 0.0209 mmol, 13% yield), ethyl 3-[3-[1-[10-[4,6-difluoro-1-(2-trimethylsilylethoxymethyl)indol-5-yl]oxy-6-(hydroxymethyl)-5,6-dihydro-[1,2,4]triazolo[1,5-d][1,4]benzoxazepin-2-yl]ethyl]-2-fluoro-phenyl]propanoate—product A (23 mg, 0.0312 mmol, 20% yield), ethyl 3-[3-[1-[10-[4,6-difluoro-1-(2-trimethylsilylethoxymethyl)indol-5-yl]oxy-6-methylene-5H-[1,2,4]triazolo[1,5-d][1,4]benzoxazepin-2-yl]ethyl]-2-fluoro-phenyl]propanoate (12 mg, 0.0167 mmol, 10% yield) and ethyl 3-[3-[1-[14-[4,6-difluoro-1-(2-trimethylsilylethoxymethyl)indol-5-yl]oxy-8-hydroxy-10-oxa-3,5,6-triazatricyclo[9.4.0.02,6]pentadeca-1(15),2,4,11,13-pentaen-4-yl]ethyl]-2-fluoro-phenyl]propanoate—product B (5.0 mg, 0.00679 mmol, 4% yield).

### LC-MS Method:

Mobile Phase: A: water (0.01% TFA), B: ACN (0.01% TFA)
Gradient: 5% to 95% B within 1.3 min, 95% B for 1.7 min
Column: Sunfire C18, 4.6*50 mm, 3.5 μm
Flow Rate: 1.8 mL/min; Column Temperature: 50° C.
Product A: MS (ESI): 737.3 m/z (M+H)⁺ at 2.25 min
Product B: MS (ESI): 737.3 m/z (M+H)⁺ at 2.29 min

### 3-[3-[1-[10-[(4,6-Difluoro-1H-indol-5-yl)oxy]-6-(hydroxymethyl)-5,6-dihydro-[1,2,4]triazolo[1,5-d][1,4]benzoxazepin-2-yl]ethyl]-2-fluoro-phenyl]propanoic acid

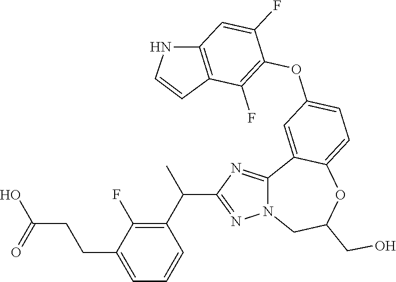 [SMILES: CC(c1nc2n(n1)CC(CO)Oc1ccc(Oc3c(F)cc4[nH]ccc4c3F)cc1-2)c1cccc(CCC(=O)O)c1F]

Step D: To a stirred solution of ethyl 3-[3-[1-[10-[4,6-difluoro-1-(2-trimethylsilylethoxymethyl)indol-5-yl]oxy-6-(hydroxymethyl)-5,6-dihydro-[1,2,4]triazolo[1,5-d][1,4]benzoxazepin-2-yl]ethyl]-2-fluoro-phenyl]propanoate (1.00 eq, 23 mg, 0.0312 mmol) in TBAF (1.00 eq, 2.0 mL, 0.0312 mmol) was added ethylenediamine (1.50 eq, 0.0032 mL, 0.0468 mmol). The mixture was stirred at 70° C. for 16 hours. To the mixture was added aqueous hydrochloric acid (1 M) to adjust the pH to 2, followed by extraction with ethyl acetate (50 mL×3). The combined organic extracts were washed with brine (20 mL), dried over anhydrous sodium sulfate, filtered, and concentrated. The residue was purified by prep-HPLC to give 3-[3-[1-[10-[(4,6-difluoro-1H-indol-5-yl)oxy]-6-(hydroxymethyl)-5,6-dihydro-[1,2,4]triazolo[1,5-d][1,4]benzoxazepin-2-yl]ethyl]-2-fluoro-phenyl]propanoic acid (16 mg, 0.0271 mmol, 87% yield). MS (ESI): 579.2 m/z (M+H)⁺

### Examples 13 and 14

### Synthesis of diastereomers of 3-[3-[1-[14-[(4,6-difluoro-1H-indol-5-yl)oxy]-8-hydroxy-10-oxa-3,5,6-triazatricyclo[9.4.0.02,6]pentadeca-1(15),2,4,11,13-pentaen-4-yl]ethyl]-2-fluoro-phenyl]propanoic acid

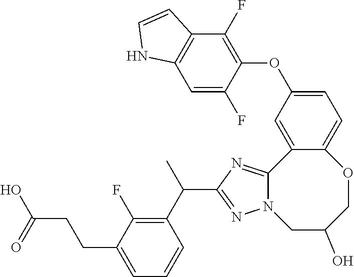 [SMILES: CC(c1nc2n(n1)CC(O)COc1ccc(Oc3c(F)cc4[nH]ccc4c3F)cc1-2)c1cccc(CCC(=O)O)c1F]

Step A: To a solution of ethyl 3-[3-[1-[14-[4,6-difluoro-1-(2-trimethylsilylethoxymethyl)indol-5-yl]oxy-8-hydroxy-10-oxa-3,5,6-triazatricyclo[9.4.0.02,6]pentadeca-1(15),2,4,11,13-pentaen-4-yl]ethyl]-2-fluoro-phenyl]propanoate (product B from Step C, Example 12) (1.00 eq, 7.0 mg, 0.00950 mmol) in TBAF (1.00 eq, 2.0 mL, 0.00950 mmol) was added ethylenediamine (1.50 eq, 0.00096 mL, 0.0142 mmol). The mixture was stirred at 70° C. for 16 hours. To the mixture was added hydrochloric acid (1 M) to adjust the pH to 2, followed by extraction with ethyl acetate (50 mL×3). The combined organic extracts were washed with brine (20 mL), dried over anhydrous sodium sulfate, filtered, and concentrated. The residue was purified by prep-HPLC to give Diastereomer 1 (2.0 mg, 0.00346 mmol, 36% yield) and Diastereomer 2 (2.7 mg, 0.00467 mmol, 49% yield) of 3-[3-[1-[14-[(4,6-difluoro-1H-indol-5-yl)oxy]-8-hydroxy-10-oxa-3,5,6-triazatricyclo[9.4.0.02,6]pentadeca-1(15),2,4,11,13-pentaen-4-yl]ethyl]-2-fluoro-phenyl]propanoic acid.

### LC-Mass Method:

Mobile Phase: A: water (0.01% TFA), B: ACN (0.01% TFA)
Gradient from 5 to 95% of B in 1.4 min at 2.0 mL/min
Column: SunFire C18 (4.6×50 mm, 3.5 μm); Column Temperature: 50° C.
Diastereomer 1: MS (ESI): 579.2 m/z (M+H)⁺ at 1.83 min
Diastereomer 2: MS (ESI): 579.2 m/z (M+H)⁺ at 1.86 min

### Examples 15 and 16

### Synthesis of enantiomers of 3-[3-[1-[10-[(4,6-difluoro-1H-indol-5-yl)oxy]-5,6-dihydro-[1,2,4]triazolo[1,5-d][1,4]benzoxazepin-2-yl]ethyl]-2-fluoro-phenyl]propanoic acid

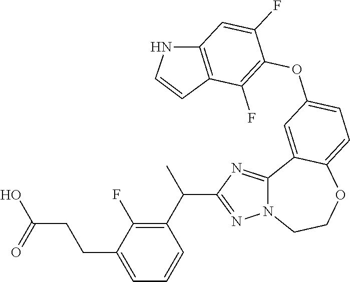 [SMILES: CC(c1nc2n(n1)CCOc1ccc(Oc3c(F)cc4[nH]ccc4c3F)cc1-2)c1cccc(CCC(=O)O)c1F]

### Ethyl 3-[3-[1-[5-[5-[4,6-difluoro-1-(2-trimethylsilylethoxymethyl)indol-5-yl]oxy-2-(methoxymethoxy)phenyl]-1-(2-oxoethyl)-1,2,4-triazol-3-yl]ethyl]-2-fluoro-phenyl]propanoate

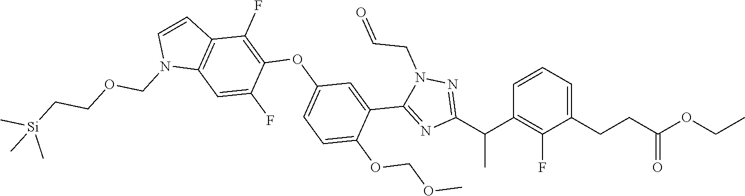 [SMILES: CCOC(=O)CCc1cccc(C(C)c2nc(-c3cc(Oc4c(F)cc5c(ccn5COCC[Si](C)(C)C)c4F)ccc3OCOC)n(CC=O)n2)c1F]

Step A: To a stirred solution of ethyl 3-[3-[1-[5-[5-[4,6-difluoro-1-(2-trimethylsilylethoxymethyl)indol-5-yl]oxy-2-(methoxymethoxy)phenyl]-1-(2,3-dihydroxypropyl)-1,2,4-triazol-3-yl]ethyl]-2-fluoro-phenyl]propanoate (1.00 eq, 1.30 g, 1.63 mmol) in THF (15 mL) and water (3 mL) was added sodium periodate (3.00 eq, 1044 mg, 4.88 mmol). The mixture was stirred at room temperature for 16 hours, diluted with water (200 mL), and extracted with ethyl acetate (80 mL×3). The combined organic extracts were washed with brine (50 mL), dried over anhydrous sodium sulfate, filtered, and concentrated to afford a crude product. MS (ESI): 767.3 m/z (M+H)⁺. The crude product (1 g) was used directly in the next step.

### Ethyl 3-[3-[1-[5-[5-[4,6-difluoro-1-(2-trimethylsilylethoxynethyl)indol-5-yl]oxy-2-(methoxymethoxy)phenyl]-1-(2-hydroxyethyl)-1,2,4-triazol-3-yl]ethyl]-2-fluoro-phenyl]propanoate

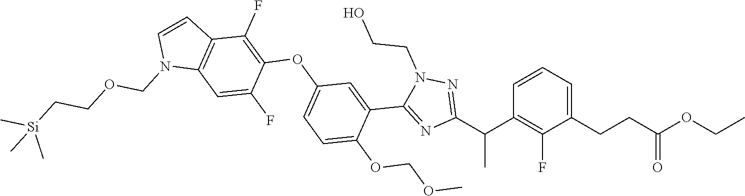 [SMILES: CCOC(=O)CCc1cccc(C(C)c2nc(-c3cc(Oc4c(F)cc5c(ccn5COCC[Si](C)(C)C)c4F)ccc3OCOC)n(CCO)n2)c1F]

Step B: To a stirred solution of ethyl 3-[3-[1-[5-[5-[4,6-difluoro-1-(2-trimethylsilylethoxymethyl)indol-5-yl]oxy-2-(methoxymethoxy)phenyl]-1-(2-oxoethyl)-1,2,4-triazol-3-yl]ethyl]-2-fluoro-phenyl]propanoate (1.00 eq, 1.00 g, 1.30 mmol) in methanol (10 mL) was added sodium borohydride (3.00 eq, 148 mg, 3.91 mmol). The mixture was stirred at room temperature for 2 hours, diluted with water (100 mL), and extracted with ethyl acetate (30 mL×3). The combined organic extracts were washed with brine (30 mL), dried over anhydrous sodium sulfate, filtered, and concentrated. The crude product was purified by column chromatography on silica, eluting with 0-45% ethyl acetate in petroleum ether, to afford ethyl 3-[3-[1-[5-[5-[4,6-difluoro-1-(2-trimethylsilylethoxymethyl)indol-5-yl]oxy-2-(methoxymethoxy)phenyl]-1-(2-hydroxyethyl)-1,2,4-triazol-3-yl]ethyl]-2-fluoro-phenyl]propanoate (300 mg, 0.390 mmol, 30% yield). MS (ESI): 769.3 m/z (M+H)⁺

### 3-[3-[1-[5-[5-[(4,6-Difluoro-1H-indol-5-yl)oxy]-2-(methoxymethoxy)phenyl]-1-(2-hydroxyethyl)-1,2,4-triazol-3-yl]ethyl]-2-fluoro-phenyl]propanoic acid

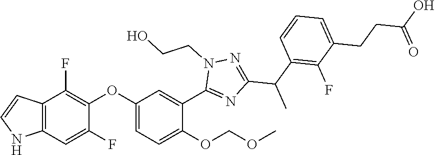 [SMILES: COCOc1ccc(Oc2c(F)cc3[nH]ccc3c2F)cc1-c1nc(C(C)c2cccc(CCC(=O)O)c2F)nn1CCO]

Step C: To a solution of ethyl 3-[3-[1-[10-[4,6-difluoro-1-(2-trimethylsilylethoxymethyl)indol-5-yl]oxy-6-(hydroxymethyl)-5,6-dihydro-[1,2,4]triazolo[1,5-d][1,4]benzoxazepin-2-yl]ethyl]-2-fluoro-phenyl]propanoate (1.00 eq, 23 mg, 0.0312 mmol) in TBAF (1.00 eq, 2.0 mL, 0.0312 mmol) was added ethylenediamine (1.50 eq, 0.0032 mL, 0.0468 mmol). The mixture was stirred at 70° C. for 16 hours. To the mixture was added hydrochloric acid (1 M) to adjust the pH to 2, followed by extraction with ethyl acetate (50 mL×3). The combined organic extracts were washed with brine (20 mL), dried over anhydrous sodium sulfate, filtered and concentrated to give 3-[3-[1-[5-[5-[(4,6-difluoro-1H-indol-5-yl)oxy]-2-(methoxymethoxy)phenyl]-1-(2-hydroxyethyl)-1,2,4-triazol-3-yl]ethyl]-2-fluoro-phenyl]propanoic acid (178 mg, 0.292 mmol, 75% yield). MS (ESI): 611.3 m/z (M+H)⁺

### Methyl 3-[3-[1-[5-[5-[(4,6-difluoro-1H-indol-5-yl)oxy]-2-hydroxy-phenyl]-1-(2-hydroxyethyl)-1,2,4-triazol-3-yl]ethyl]-2-fluoro-phenyl]propanoate

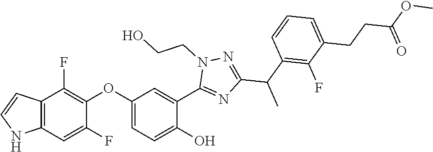 [SMILES: COC(=O)CCc1cccc(C(C)c2nc(-c3cc(Oc4c(F)cc5[nH]ccc5c4F)ccc3O)n(CCO)n2)c1F]

Step D: To a stirred solution of 3-[3-[1-[5-[5-[(4,6-difluoro-1H-indol-5-yl)oxy]-2-(methoxymethoxy)phenyl]-1-(2-hydroxyethyl)-1,2,4-triazol-3-yl]ethyl]-2-fluoro-phenyl]propanoic acid (1.00 eq, 178 mg, 0.292 mmol) in methanol (254 eq, 3.0 mL, 74.2 mmol) was added sulfuric acid (19.3 eq, 0.30 mL, 5.63 mmol). The mixture was stirred at 50° C. for 1 hour, diluted with water (100 mL), and extracted with ethyl acetate (50 mL×2). The combined organic extracts were washed with brine (20 mL×2), dried over anhydrous sodium sulfate, filtered, and concentrated. The residue was purified by flash chromatography on silica, eluting with 0-83% ethyl acetate in petroleum ether, to give methyl 3-[3-[1-[5-[5-[(4,6-difluoro-1H-indol-5-yl)oxy]-2-hydroxy-phenyl]-1-(2-hydroxyethyl)-1,2,4-triazol-3-yl]ethyl]-2-fluoro-phenyl]propanoate (120 mg, 0.207 mmol, 71% yield). MS (ESI): 581.3 n/z (M+H)⁺

### Methyl 3-[3-[1-[10-[(4,6-difluoro-1H-indol-5-yl)oxy]-5,6-dihydro-[1,2,4]triazolo[1,5-d][1,4]benzoxazepin-2-yl]ethyl]-2-fluoro-phenyl]propanoate

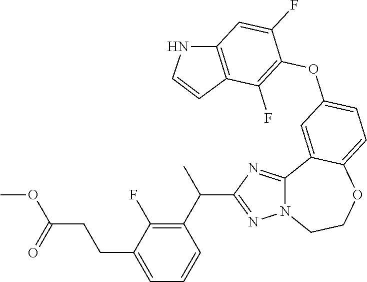 [SMILES: COC(=O)CCc1cccc(C(C)c2nc3n(n2)CCOc2ccc(Oc4c(F)cc5[nH]ccc5c4F)cc2-3)c1F]

Step E: To a stirred solution of methyl 3-[3-[1-[5-[5-[(4,6-difluoro-1H-indol-5-yl)oxy]-2-hydroxy-phenyl]-1-(2-hydroxyethyl)-1,2,4-triazol-3-yl]ethyl]-2-fluoro-phenyl]propanoate (1.00 eq, 120 mg, 0.207 mmol) in toluene (12 mL) was added under N2 cyanomethylenetributylphosphorane (6.00 eq, 299 mg, 1.24 mmol). The mixture was stirred at 150° C. in a microwave oven for 1 hour. The mixture was diluted with water (300 mL) and extracted with ethyl acetate (100 mL×3). The combined organic extracts were washed with brine (50 mL×2), dried over anhydrous sodium sulfate, filtered, and concentrated. The residue was purified by flash chromatography on silica, eluting with 0-42% ethyl acetate in petroleum ether, to give methyl 3-[3-[1-[10-[(4,6-difluoro-1H-indol-5-yl)oxy]-5,6-dihydro-|1,2,4|triazolo|1,5-d∥1,4|benzoxazepin-2-yl|ethyl|-2-fluoro-phenyl|propanoate (60 mg, 0.107 mmol, 52% yield). MS (ESI): 563.1 m/z (M+H)⁺

### 3-[3-[1-[10-[(4,6-Difluoro-1H-indol-5-yl)oxy]-5,6-dihydro-[1,2,4]triazolo[1,5-d][1,4]benzoxazepin-2-yl]ethyl]-2-fluoro-phenyl]propanoic acid

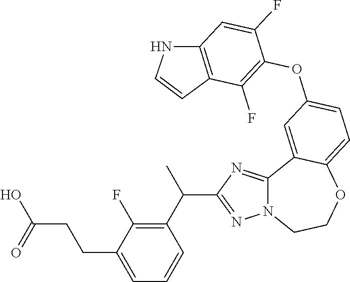 [SMILES: CC(c1nc2n(n1)CCOc1ccc(Oc3c(F)cc4[nH]ccc4c3F)cc1-2)c1cccc(CCC(=O)O)c1F]

Step F: To a stirred solution of methyl 3-[3-[1-[10-[(4,6-difluoro-1H-indol-5-yl)oxy]-5,6-dihydro-[1,2,4]triazolo[1,5-d][1,4]benzoxazepin-2-yl]ethyl]-2-fluoro-phenyl]propanoate (1.00 eq, 50 mg, 0.0889 mmol) in tetrahydrofuran (2 mL) was added lithium hydroxide (2.00 mL, 1M in water). The reaction was stirred at room temperature for 6 hours, acidified with hydrochloric acid (1 M in water, 15 mL), and extracted with ethyl acetate (30 mL). The separated organic layer was washed with brine (20 mL×2), dried over anhydrous sodium sulfate, filtered, and concentrated. The residue was purified by prep-HPLC to give 3-[3-[1-[10-[(4,6-difluoro-1H-indol-5-yl)oxy]-5,6-dihydro-[1,2,4]triazolo[1,5-d][1,4]benzoxazepin-2-yl]ethyl]-2-fluoro-phenyl]propanoic acid (30 mg, 0.0547 mmol, 62% yield).

The product racemic mixture was separated by chiral SFC to give Enantiomer 1 of 3-[3-[1-[10-[(4,6-difluoro-1H-indol-5-yl)oxy]-5,6-dihydro-[1,2,4]triazolo[1,5-d][1,4]benzoxazepin-2-yl]ethyl]-2-fluoro-phenyl]propanoic acid (11 mg, 0.0204 mmol, 37% yield) and Enantiomer 2 of 3-[3-[1-[10-[(4,6-difluoro-1H-indol-5-yl)oxy]-5,6-dihydro-[1,2,4]triazolo[1,5-d][1,4]benzoxazepin-2-yl]ethyl]-2-fluoro-phenyl]propanoic acid (12 mg, 0.0219 mmol, 40% yield) as white solids.

### Chiral Preparatory Chromatography Conditions:

Instrument: SFC-150 (Waters); Detection wavelength: 214 nm
Column: (R,R)Whelk-O1 4.6*100 mm 3.5 μm; Column temperature: 40° C.
Flow rate: 3.0 mL/min. Back pressure: 2000 psi; Cycle time: 2.73 min
Mobile phase: MeOH spiked with 0.2% NH₃ (7M in MeOH).
Sample solution: 30 mg dissolved in 15 ml methanol; Injection volume: 5.00 μl.
Enantiomer 1: MS (ESI): 549.3 m/z (M+H)⁺
Enantiomer 2: MS (ESI): 549.3 m/z (M+H)⁺

### Biological Assays

### Example 17. Aggregation Analysis Using Differential Static Light Scattering (DSLS)

Purified recombinant NBD1 was produced using previously described methods (A. Schmidt, J. L. Mendoza, P. J. Thomas (2011) Biochemical and Biophysical Approaches to Probe CFTR Structure (365-376) M. D. Amaral, K. Kunzelmann (eds.), Cystic Fibrosis, Methods in Molecular Biology 741, Springer Science+Business Media). The effect of test compounds on thermal stability of NBD1 was evaluated by differential static light scattering (DSLS) using the Harbinger Stargazer-384 instrument (Epiphyte Three, Toronto, Canada). Test compounds were dissolved and diluted to desired concentrations in 100% DMSO. The compounds or DMSO controls (100 nL) were stamped into wells of a 385-well low volume optical plate (Corning Inc., Corning, NY) using the Echo 555 acoustic liquid handler (Labcyte Inc., San Jose, CA).

NBD1 protein was diluted to 0.2 mg/ml in S200 buffer (50 mM Tris-HCl, 150 mM NaCl, 5 mM MgCl₂, 2 mM ATP, 2 mM DTT, pH7.6) containing 1% glycerol. 10 uL of protein solution was aliquoted into the 384-well plate harboring the test compounds and 10 uL mineral oil was overlayed onto the protein solution, using the epMotion robotic liquid handler (Eppendorf North America, Hauppauge, NY). After placing into the Stargazer instrument, the plate was heated at 1° C. per minute to 70° C. Images were captured from 25° C. to 70° C. every 0.5° C. At the end of the experiment run, instrument software integrated image files and analyzed data automatically. A linear regression curve was generated for each well, representing the increase in light scattering over time. A temperature of aggregation (Tagg) was calculated based on the inflection point of the curve. To better compare data across experiments the average Tagg for DMSO control wells was calculated and subtracted from values for wells containing compounds to obtain a “ΔTagg” value. These ΔTagg values reflect stabilizing efficacy of the compounds.

Data for Compounds 1-9 are provided in Table 2 below.

TABLE 2

| | DSLS NBD1/3D |
|---|---|
| | Human Stability |
| | delta-T (° C.) |
| Example No | @ 100 (μM) |
| Example 1 | A |
| Example 2 | A |
| Example 3 | A |
| Example 4 | B |
| Example 5 | B |
| Example 6 | A |
| Example 7 | A |
| Example 8 | A |
| Example 9 | B |
| Example 10 | C |
| Example 11 | A |
| Example 12 | A |
| Example 13 | A |
| Example 14 | A |
| Example 15 | A |
| Example 16 | C |

*A: T ≥ 8° C.; B: 4-8° C.; C: ≤ 4° C.

### Example 18. TECC24 AUC Fold Over DMSO @10 μM

The effects of a test agent on CFTR-mediated transepithelial chloride transport was measured using TECC24 recording analysis. Test agents were solubilized in DMSO. Solubilized test agents were mixed with incubation medium containing DMEM/F12, Ultroser G (2%; Crescent Chemical, catalog #67042), Hyclone Fetal Clone II (2%; GE Healthcare, catalog #SH30066.02), bovine brain extract (0.25%; Lonza, catalog #CC-4098), insulin (2.5 g/mL), IL-13 (10 ng/mL), hydrocortisone (20 nM), transferrin (2.5 g/mL), triiodothyronine (500 nM), ethanolamine (250 nM), epinephrine (1.5 μM), phosphoethanolamine (250 nM), and retinoic acid (10 nM). Primary human bronchial epithelial cells from a ΔF508 homozygous CF donor (CF-HBE cells; from University of North Carolina Cystic Fibrosis Tissue Procurement Center), grown on Transwell HTS 24-well cell culture inserts (Costar, catalog #3378), were exposed to test agents or controls dissolved in incubation medium. The CF-HBE cells were cultured at 36.5° C. for 48 hours before TECC24 recordings were performed in the presence or absence of test agent, a positive control or vehicle (DMSO).

Following incubation, the transwell cell culture inserts containing the test agent or control-treated CF-HBE cells were loaded onto a TECC24 apparatus (TECC v7 or MTECC v2; EP Design) to record the transepithelial voltage (VT) and resistance (TEER) using 4 AgCl electrodes per well configured in current-clamp mode. The apical and basolateral bath solutions both contained (in mM) 140 NaCl, 5 KCl, 2 CaCl₂), 1 MgCl₂, 10 Hepes, and 10 glucose (adjusted to pH 7.4 with NaOH). To inhibit basal Na+ absorption, the ENaC inhibitor benzamil (10 μM) was added to the bath. Then, the adenylate cyclase activator, forskolin (10 μM), was added to the bath to activate CFTR. The forskolin-stimulated Cl—transport was halted by addition of CFTR inhibitor-172 (20 μM) to the bath at the end of the experiment to confirm specificity. VT and TEER recordings were digitally acquired at routine intervals using TECC or MTECC software (EP Design). VT and TEER were transformed into equivalent transepithelial Cl—current (IEQ), and the Area Under the Curve (AUC) of the IEQ time course between forskolin and CFTR inhibitor-172 addition is generated using Excel (Microsoft). Efficacy is expressed as the ratio of the test agent AUC divided by vehicle AUC. EC50s based on AUC are generated using the non-linear regression log(agonist) vs. response function in Prism software (Graphpad) with HillSlope fixed=1.

If a test agent increased the AUC of the forskolin-stimulated IEQ relative to vehicle in CF-HBE cells, and this increase was inhibited by CFTR inhibitor-172, then the test agent was considered a CFTR corrector. The data is shown in Table 3 below.

TABLE 3

| | TECC24 @ |
|---|---|
| Example No. | 10 uM over DMSO |
| Example 3 | A |
| Example 4 | B |
| Example 5 | C |
| Example 6 | A |
| Example 10 | C |
| Example 11 | C |
| Example 13 | C |
| Example 14 | B |
| Example 15 | A |

ND = Not determined; “A” refers to AUC @10 uM >= 12; “B” refers to AUC @ 10 uM between 4-12; “C” refers to AUC @10 uM <= 4.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 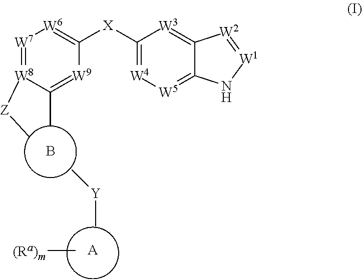
or a pharmaceutically acceptable salt thereof
wherein
W¹ is selected from the group consisting of —C(H)═, and —N═;
W² is selected from the group consisting of —C(H)═, —C(R^{d})═, and —N═;
W³ is selected from the group consisting of —C(H)═, —C(R^{d})═, and —N═;
W⁴ is selected from the group consisting of —C(H)═, —C(R^{d4})═, and —N═;
W⁵ is selected from the group consisting of —C(H)═, —C(R^{d5})═, and —N═;
W⁶ is selected from the group consisting of —C(H)═, —C(R^{c})═, and —N═;
W⁷ is selected from the group consisting of —C(H)═, —C(R^{c})═, and —N═;
W⁸ is selected from the group consisting of C or N;
W⁹ is selected from the group consisting of —C(H)═, —C(R^{c9})═, and —N═;
Ring A is optionally substituted phenyl or optionally substituted 5-6-membered heteroaryl;
Ring B is optionally substituted 5-member heteroaryl, wherein R^{b} is optionally substituted with n instances of R^{b};
each R^{a} is independently selected from the group consisting of halogen, oxo —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein each R^{a} is independently substituted with 0-4 instances of R^{aa},
each R^{aa} is independently selected from the group consisting of halogen, —COOH, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —SO₂R¹, —SO(NR²)R¹, —SO₂N(R²)R¹, —N(H)C(O)R¹, and —N(H)C(O)N(R¹)₂,
wherein two instances of R^{aa} are optionally taken together with any intervening atoms to form an optionally substituted 4-6 membered heterocyclyl ring;
each R^{b} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{c} is independently selected from the group consisting of halogen, oxo —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R^{d} is independently selected from the group consisting of halogen, oxo, —CN, —NO₂—OR¹, —SR¹, —N(R¹)₂, —C(O)OR¹, C(O)N(R¹)₂, —N(H)C(O)R¹, —SO₂R¹, —SO(NR²)R¹, —SO₂N(R²)R¹, —N(H)C(O)N(R¹)₂, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
R^{c9} is halogen;
R^{d5} is halogen;
R^{d5} is halogen;
each R¹ is independently selected from the group consisting of hydrogen, —(CH₂)₁₋₃R², —C(O)R², —(CH₂)₁₋₃OR², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
wherein two instances of R¹ are optionally taken together with any intervening atoms to form an optionally substituted 3-7 membered heterocyclyl ring;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
X is selected from the group consisting of —O—, —S—, —S(O)—, —S(O)₂—, —SO(NR²)—, —N(R¹)—, —C(R¹)(R²)—, —C(O)—, and —CH(OH)—;
Y is selected from the group consisting of optionally substituted C₁-C₃ alkylene, —O—, —S—, —S(O)—, —SO(NR²)—, and —S(O)₂—;
Z is optionally substituted C₁-C₄ alkylene, wherein one methylene unit is optionally replaced with —O—, —N(R¹)—, S—, —S(O)—, or —S(O)₂—;
n is 0, 1, or 2; and
m is 0, 1, 2, or 3.

2. The compound of claim 1, wherein W¹ is —N═.

3. The compound of claim 1, wherein W² is —N═.

4. The compound of claim 1, wherein W³ is —N═.

5. The compound of claim 1, wherein W⁴ is —N═.

6. The compound of claim 1, wherein the compound is of Formula (I-a), (I-b), (I-c), (I-d), (I-e), or (I-f): 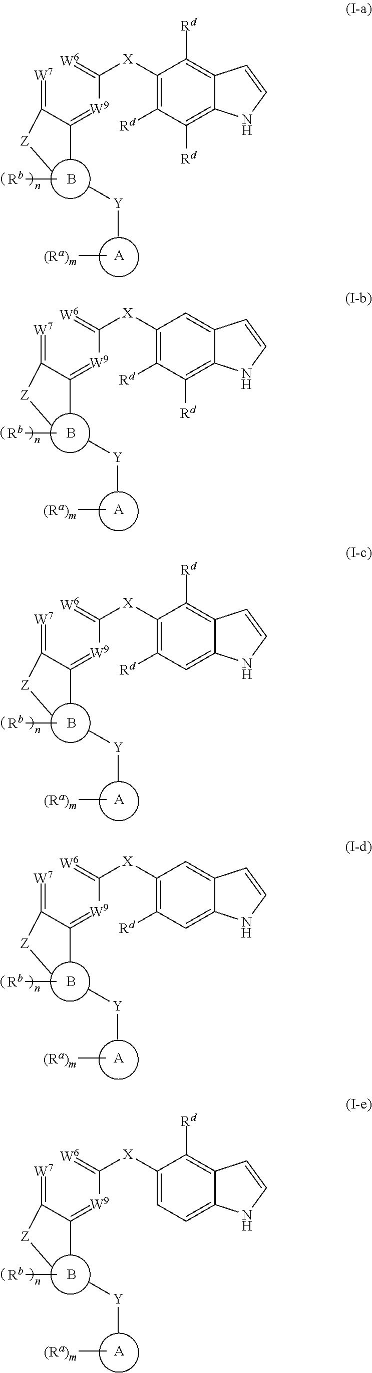 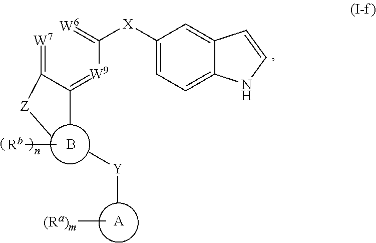
or a pharmaceutically acceptable salt thereof.

7. The compound of any of claims 1-6, wherein
W⁶ is —C(H)═;
W⁷ is —C(H)═;
W⁸ is C; and
W⁹ is —N═.

8. The compound of any of claims 1-6, wherein
W⁶ is —C(R^{c})═;
W⁷ is —C(H)═;
W⁸ is C; and
W⁹ is —C(H)═.

9. The compound of any of claims 1-6, wherein
W⁶ is —C(H)═;
W⁷ is —C(H)═; and
W⁸ is —C═; and
W⁹ is —C(H)═.

10. The compound of any of claims 1-9, wherein Ring A is selected from the group consisting of thiophenyl, pyrazolyl, pyrrolyl, and thiazolyl.

11. The compound of claim 10, wherein Ring A is selected from the group consisting of

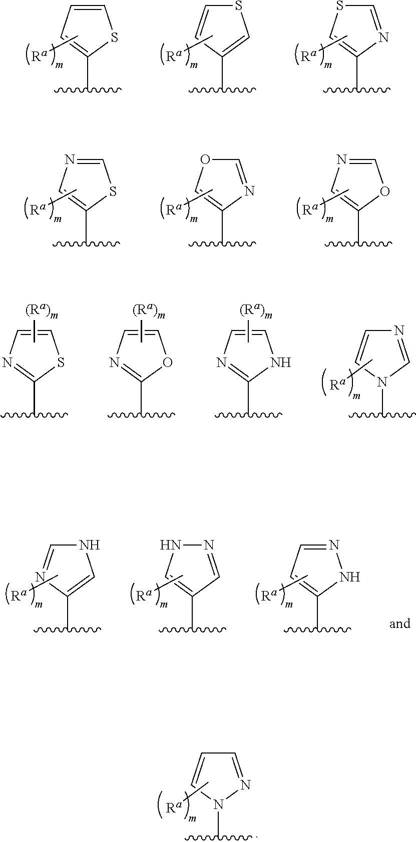

12. The compound of claim 10, wherein Ring A is selected from the group consisting of

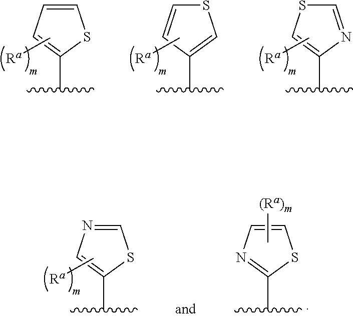

13. The compound of any of claims 1-9, wherein Ring A is optionally substituted phenyl.

14. The compound of any of claims 1-9, wherein Ring A is optionally substituted pyridinyl.

15. The compound of any of claims 1-14, wherein Ring B is optionally substituted 5-member heteroaryl selected from the group consisting of pyrrolyl, pyrazolyl, imidazolyl, triazolyl, furanyl, oxazolyl, thiophenyl, and thiazolyl.

16. The compound of claim 15, wherein Ring B is imidazolyl.

17. The compound of claim 15, wherein Ring B is selected from the group consisting of

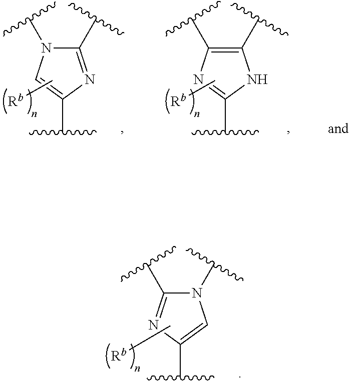

18. The compound of claim 15, wherein Ring B is pyrazolyl.

19. The compound of claim 15, wherein Ring B is selected from the group consisting of

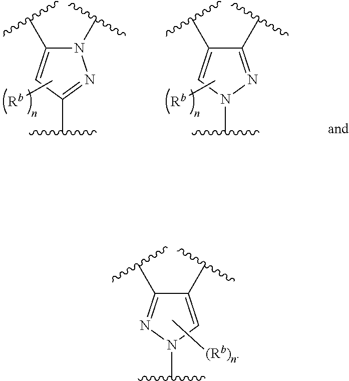

20. The compound of claim 15, wherein Ring B is oxazolyl.

21. The compound of claim 15, wherein Ring B is

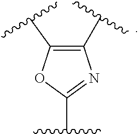 [SMILES: *c1nc(*)c(*)o1]

22. The compound of claim 15, wherein Ring B is thiazolyl.

23. The compound of claim 15, wherein Ring B is

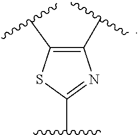 [SMILES: *c1nc(*)c(*)s1]

24. The compound of claim 15, wherein Ring B is

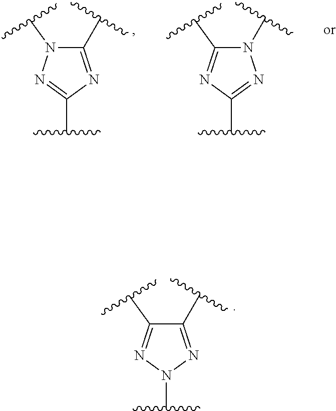 [SMILES: *c1nc(*)n(*)n1 ; *c1nc(*)n(*)n1 ; *c1nn(*)nc1*]

25. The compound of claim 15, wherein Ring B is

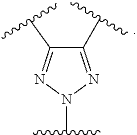 [SMILES: *c1nn(*)nc1*]

26. The compound of claim 15, wherein the compound is of Formula (II): 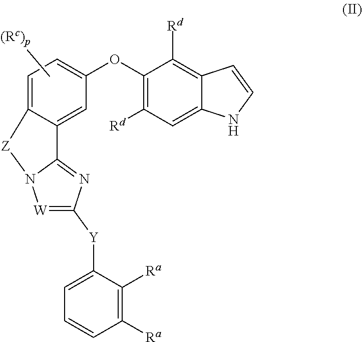
or a pharmaceutically acceptable salt thereof, wherein W is ═N— or ═C(H)—.

27. The compound of claim 15 or 26, wherein W is —C(H)═.

28. The compound of claim 15 or 26, wherein W is —N═.

29. The compound of any of claims 1-28, wherein Y is —S—.

30. The compound of any of claims 1-28, wherein Y is optionally substituted C₁-C₃ alkylene.

31. The compound of claim 30, wherein Y is —CH₂—.

32. The compound of claim 30, wherein Y is —C(CH₃)H—

33. The compound of any of claims 1-32, wherein Z is optionally substituted C₁-C₄ alkylene.

34. The compound of claim 33, wherein Z is —CH₂—.

35. The compound of claim 9, wherein Z is —CH₂CH₂—.

36. The compound of claim 33, wherein Z is —CH₂CH₂CH₂—.

37. The compound of any of claims 1-32, wherein Z is optionally substituted C₁-C₄ alkylene, wherein one methylene unit is replaced with —O—.

38. The compound of claim 37, wherein Z is —CH₂CH₂O—.

39. The compound of any of claims 1-32, wherein Z is optionally substituted C₃-C₄ alkenylene.

40. The compound of claim 39, wherein Z is —C(H)═C(H)—.

41. The compound of any of claims 1-40, wherein each R^{a} is independently selected from halogen or optionally substituted C₁-C₆ alkyl, wherein each R^{a} is independently substituted with 0-4 instances of R^{aa}.

42. The compound of claim 41, wherein each R^{a} is selected from the group consisting of independently fluoro, chloro, —CH₂CH₂COOH, —CH₂CH(Me)CO₂H and —CH₂CH(OH)CH₂(OH).

43. The compound of any of claims 1-42, wherein R^{d} is halogen.

44. The compound of claim 43, wherein R^{d} is fluoro.

45. The compound of any of claims 1-44, wherein R^{d4} is halogen.

46. The compound of claim 45, wherein R^{d4} is fluoro.

47. The compound of any of claims 1-46, wherein R^{d5} is halogen.

48. The compound of claim 47, wherein R^{d}s is fluoro.

49. A compound selected from the group consisting of

| Compound No. | Structure |
|---|---|
| 1 | 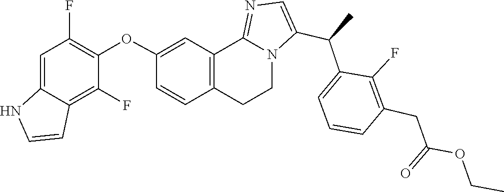 [SMILES: CCOC(=O)Cc1cccc([C@H](C)c2cnc3n2CCc2ccc(Oc4c(F)cc5[nH]ccc5c4F)cc2-3)c1F] |
| 2 | 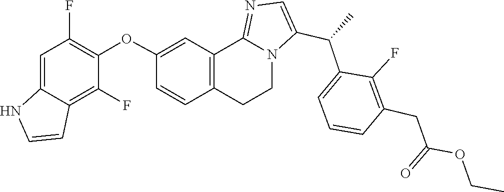 [SMILES: CCOC(=O)Cc1cccc([C@@H](C)c2cnc3n2CCc2ccc(Oc4c(F)cc5[nH]ccc5c4F)cc2-3)c1F] |
| 3 | 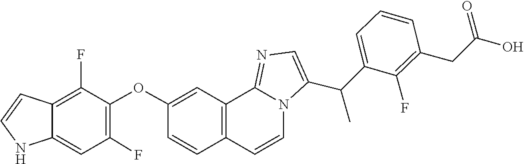 [SMILES: CC(c1cccc(CC(=O)O)c1F)c1cnc2c3cc(Oc4c(F)cc5[nH]ccc5c4F)ccc3ccn12] |
| 4 | 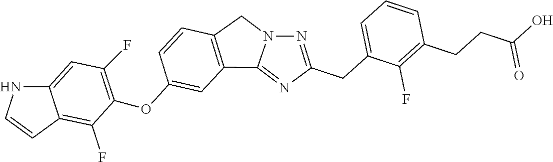 [SMILES: O=C(O)CCc1cccc(Cc2nc3n(n2)Cc2ccc(Oc4c(F)cc5[nH]ccc5c4F)cc2-3)c1F] |
| 5 | 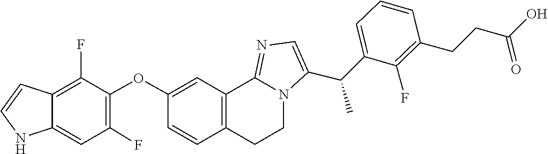 [SMILES: C[C@@H](c1cccc(CCC(=O)O)c1F)c1cnc2n1CCc1ccc(Oc3c(F)cc4[nH]ccc4c3F)cc1-2] |
| 6 | 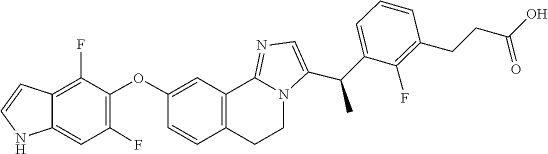 [SMILES: C[C@H](c1cccc(CCC(=O)O)c1F)c1cnc2n1CCc1ccc(Oc3c(F)cc4[nH]ccc4c3F)cc1-2] |
| 7 | 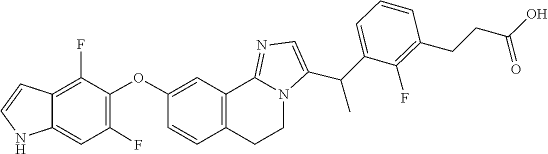 [SMILES: CC(c1cccc(CCC(=O)O)c1F)c1cnc2n1CCc1ccc(Oc3c(F)cc4[nH]ccc4c3F)cc1-2] |
| 8 | 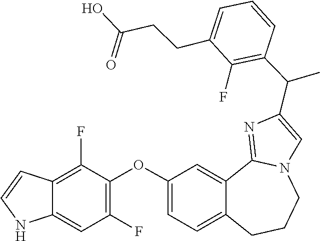 [SMILES: CC(c1cn2c(n1)-c1cc(Oc3c(F)cc4[nH]ccc4c3F)ccc1CCC2)c1cccc(CCC(=O)O)c1F] |
| 9 | 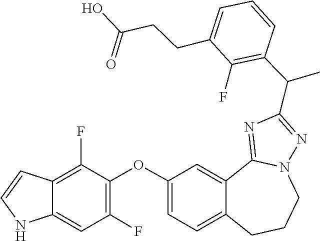 [SMILES: CC(c1nc2n(n1)CCCc1ccc(Oc3c(F)cc4[nH]ccc4c3F)cc1-2)c1cccc(CCC(=O)O)c1F] |
| 10 | 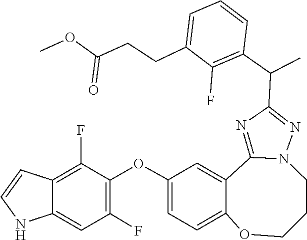 [SMILES: COC(=O)CCc1cccc(C(C)c2nc3n(n2)CCCOc2ccc(Oc4c(F)cc5[nH]ccc5c4F)cc2-3)c1F] |
| 11 | 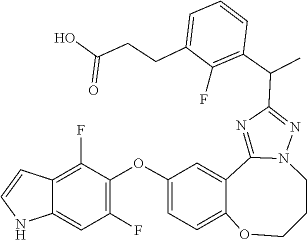 [SMILES: CC(c1nc2n(n1)CCCOc1ccc(Oc3c(F)cc4[nH]ccc4c3F)cc1-2)c1cccc(CCC(=O)O)c1F] |
| 12 | 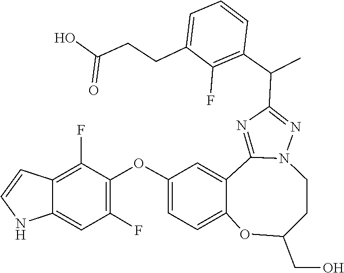 [SMILES: CC(c1nc2n(n1)CCC(CO)Oc1ccc(Oc3c(F)cc4[nH]ccc4c3F)cc1-2)c1cccc(CCC(=O)O)c1F] |
| 13 | 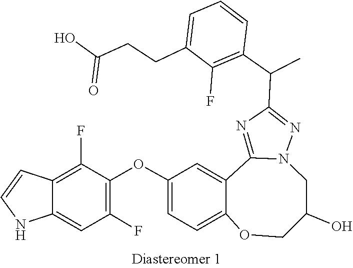 [SMILES: CC(c1nc2n(n1)CC(O)COc1ccc(Oc3c(F)cc4[nH]ccc4c3F)cc1-2)c1cccc(CCC(=O)O)c1F] |
| 14 | 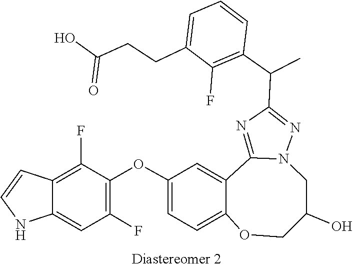 [SMILES: CC(c1nc2n(n1)CC(O)COc1ccc(Oc3c(F)cc4[nH]ccc4c3F)cc1-2)c1cccc(CCC(=O)O)c1F] |
| 15 | 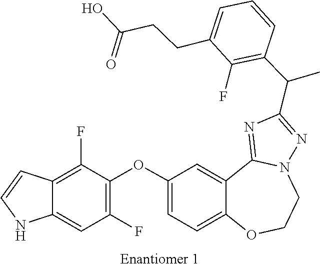 [SMILES: CC(c1nc2n(n1)CCOc1ccc(Oc3c(F)cc4[nH]ccc4c3F)cc1-2)c1cccc(CCC(=O)O)c1F] |
| 16 | 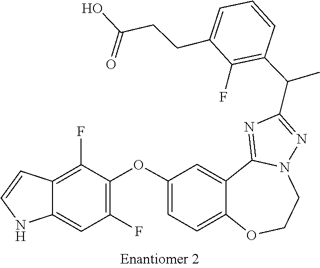 [SMILES: CC(c1nc2n(n1)CCOc1ccc(Oc3c(F)cc4[nH]ccc4c3F)cc1-2)c1cccc(CCC(=O)O)c1F] |

or a pharmaceutically acceptable salt thereof.

50. A pharmaceutical composition comprising a compound of any of the previous claims and a pharmaceutically acceptable excipient.

51. A method of treating a CFTR-mediated disease or disorder comprising administering a patient in need there of a compound any of claims 1-49 or a pharmaceutical composition of claim 50.

52. The method of claim 51, wherein the disease or condition is selected from cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, idiopathic pancreatitis, allergic bronchopulmonary aspergillosis (ABPA), liver disease, hereditary emphysema, hereditary hemochromatosis, coagulation-fibrinolysis deficiencies, protein C deficiency, Type 1 hereditary angioedema, lipid processing deficiencies, familial hypercholesterolemia, Type 1 chylomicronemia, abetalipoproteinemia, lysosomal storage diseases, I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myeloperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases, Alzheimer's disease, Parkinson's disease, amyotrophic lateral sclerosis, progressive supranuclear palsy, Pick's disease, several polyglutamine neurological disorders, Huntington's, spinocerebellar ataxia type I, spinal and bulbar muscular atrophy, dentatorubal pallidoluysian, myotonic dystrophy, spongiform encephalopathies, hereditary Creutzfeldt-Jakob disease, Fabry disease, Straussler-Scheinker syndrome, COPD, dry-eye disease, Sjogren's disease, Osteoporosis, Osteopenia, bone healing and bone growth, bone repair, bone regeneration, reducing bone resorption, increasing bone deposition, Gorham's Syndrome, chloride channelopathies, myotonia congenita, Bartter's syndrome type III, Dent's disease, hyperekplexia, epilepsy, hyperekplexia, lysosomal storage disease, Angelman syndrome, Primary Ciliary Dyskinesia (PCD), PCD with situs inversus, PCD without situs inversus and ciliary aplasia.

53. The method of claim 51 or 52, wherein the disease or condition is selected from cystic fibrosis, congenital bilateral absence of vas deferens (CBAVD), acute, recurrent, or chronic pancreatitis, disseminated bronchiectasis, asthma, allergic pulmonary aspergillosis, chronic obstructive pulmonary disease (COPD), chronic sinusitis, dry eye disease, protein C deficiency, Abetalipoproteinemia, lysosomal storage disease, type 1 chylomicronemia, mild pulmonary disease, lipid processing deficiencies, type 1 hereditary angioedema, coagulation-fibrinolyis, hereditary hemochromatosis, CFTR-related metabolic syndrome, chronic bronchitis, constipation, pancreatic insufficiency, hereditary emphysema, and Sjogren's syndrome.

54. The method of any one of claims 51-53, wherein the disease or condition is cystic fibrosis.

55. A method of treating kidney disease in a subject, comprising administering to the subject a therapeutically effective amount of a compound of any of claims 1-1 or a pharmaceutical composition of claim 50.

56. The method of claim 55, wherein the kidney disease is autosomal dominant polycystic kidney disease or autosomal recessive polycystic kidney disease.

57. The method of claim 55, wherein the kidney disease is autosomal dominant polycystic kidney disease.

58. The method of claim 55, wherein the kidney disease is autosomal recessive polycystic kidney disease.

59. A method of treating cystic fibrosis in a subject, comprising administering to the subject a therapeutically effective amount of a compound of any of claims 1-49 or a pharmaceutical composition of claim 50.

60. The method of claim 59, wherein the subject is human.
